(19) United States
(12) Patent Application Publication
(10) Pub. No.: US 2023/0372499 A1
(43) Pub. Date: Nov. 23, 2023

(54) DENDRIMER COMPOSITIONS AND METHODS FOR DRUG DELIVERY

(71) Applicants: The Johns Hopkins University, Baltimore, MD (US); Ashvattha Therapeutics, Inc., Redwood City, CA (US)

(72) Inventors: Jeffrey Cleland, Redwood City, CA (US); Rishi Sharma, Redwood City, CA (US); Minghao Sun, Redwood City, CA (US); Santiago Appiani, Redwood City, CA (US); Barbara Slusher, Baltimore, MD (US); Rana Rais, Baltimore, MD (US); Kannan Rangaramanujam, Baltimore, MD (US)

(21) Appl. No.: 17/962,297

(22) Filed: Oct. 7, 2022

Related U.S. Application Data
(63) Continuation of application No. 17/112,541, filed on Dec. 4, 2020, now abandoned.
(60) Provisional application No. 62/943,705, filed on Dec. 4, 2019.
(60) Provisional application No. 63/108,186, filed on Oct. 30, 2020.

(51) Int. Cl. A61K 47/60; A61P 35/04; A61K 47/59; A61P 35/00

(52) U.S. Cl. CPC A61K 47/60; A61P 35/04; A61K 47/595; A61P 35/00; A61K 45/06

(57) ABSTRACT

Dendrimer compositions and methods for the treatment of cancer or autoimmune diseases are described. The compositions include dendrimers complexed or conjugated with one or more active agents for the treatment or alleviation of one or more symptoms of cancer or autoimmune diseases. The dendrimers may include one or more ethylene diamine-core poly(amidoamine) (PAMAM) hydroxyl-terminated generation-4, 5, 6, 7, 8, 9, or 10 dendrimers. The active agents may be immunomodulatory agents such as STING agonists, CSF1R inhibitors, PARP inhibitors, VEGFR tyrosine kinase inhibitors, MEK inhibitors, glutaminase inhibitors, TIE II antagonists, and CXCR2 inhibitors, and STING antagonists. Methods of using the dendrimer compositions to treat cancer, bone disease or inflammatory diseases are also provided.

### CROSS-REFERENCE TO RELATED APPLICATIONS

This application is a continuation of U.S. application Ser. No. 17/112,541 filed Dec. 4, 2020, which claims benefit of U.S. Provisional Application No. 62/943,705, filed Dec. 4, 2019, and U.S. Provisional Application No. 63/108,186, filed Oct. 30, 2020, which are incorporated by reference in their entirety.

### FIELD OF THE INVENTION

The invention is generally in the field of drug delivery, and in particular, a method of delivering drugs selectively to sites or regions in need thereof.

### BACKGROUND OF THE INVENTION

The immune/inflammatory response is mostly beneficial to the host and is designed to combat pathogens and transformed cells and then reestablish homeostasis. The immune response is broadly categorized either as pro-inflammatory (including Th1 and Th17 cells, M1-activated macrophages, and pro-inflammatory mediators designed to kill pathogens or tumor cells) or as anti-inflammatory (dominated by Th2 cells, M2-activated macrophages, and anti-inflammatory cytokines, designed to repair tissue damage). Many other types of cell activation, including different types of regulatory T cells, macrophages, and B cells, are also involved in the immune/inflammatory response.

In both cancer and autoimmune diseases, an aberrant activation of the immune/inflammatory response leads to chronic diseases and accumulation of tissue damage. However, from an immunological standpoint, these two families of diseases are fundamentally different and represent opposite ways in which the immune system can go wrong. In cancer, the tumor cells are mostly unrecognized as antigens because a dominant anti-inflammatory response driven by the tumor cells suppresses anti-tumoral immune responses and promotes tumor progression and dissemination (immunosuppression). In contrast, in autoimmune diseases, self-tolerance is broken and the inflammatory response is activated in excess against the host tissue cells, which express autoantigens that are misrecognized and attacked by the immune system, leading to permanent tissue damage.

Tumor cells take advantage of immunosuppressive mechanisms and establish a strongly immunosuppressive tumor microenvironment (TME), which inhibits antitumor immune responses, supporting progression of the disease. Many cell types are thought to contribute to the generation of an immunosuppressive TME, including cancer-associated fibroblasts, myeloid-derived suppressor cells (MDSCs), regulatory T cells (Treg), and tumor-associated macrophages (TAMs).

TAMs are involved in tumor-promoting angiogenesis, fibrous stroma deposition, and metastasis. Macrophages undergo the ‘polarization’ process wherein they express different surface markers and functional programs in response to environmental stimuli such as the cytokines and other signaling mediators: classically activated macrophages (M1) produce pro-inflammatory cytokines and reactive oxygen/nitrogen species, which are crucial for host defense and tumor cell killing, and, therefore, are considered as ‘good’ macrophages; alternatively activated macrophages (M2) produce anti-inflammatory cytokines and are involved in the resolution of inflammation. Both M1- and M2-polarized macrophages have been identified in the TME.

MDSCs represent a heterogeneous population of immature myeloid cells with a strong immunosuppressive potential. They inhibit antitumor reactivity of T cells and NK cells, promote angiogenesis, establish pre-metastatic niches, and recruit other immunosuppressive cells such as regulatory T cells.

Accumulation of immunosuppressive cells at tumor tissues negatively affects clinical outcomes in cancer treatment and is associated with poor overall and progression-free survival. There remains a need for effective therapies against cancer, especially those mediated or regulated by immunosuppressive cells.

Spontaneous T cell responses against tumors occur frequently and have prognostic value in patients. The generation of a spontaneous T cell response against tumor-associated antigens depends on innate immune activation, which drives type I interferon (IFN) production. Recent work has revealed a major role for the STING pathway of cytosolic DNA sensing in this process. This cascade of events contributes to the activation of Batf3-lineage dendritic cells (DCs), which appear to be central to anti-tumor immunity. Non-T cell-inflamed tumors lack chemokines for Batf3 DC recruitment, have few Batf3 DCs, and lack a type I IFN gene signature, suggesting that failed innate immune activation may be the ultimate cause for lack of spontaneous T cell activation and accumulation (Corrales L, et al., Cell Research volume 27, pages 96-108(2017)). There is a need for new strategies for effectively triggering innate immune activation and/or Batf3 DC recruitment for optimal anti-tumor effects.

In the case of autoimmune diseases, the immune responses are usually dominated by Th1 and Th17 cells and their cytokine products IL-2, IFNγ, and IL-17 (in Th1 autoimmune diseases such as rheumatoid arthritis, RA, multiple sclerosis, MS, and Hashimoto thyroiditis, HT) or by Th2 cells and their anti-inflammatory cytokines IL-4, TGFβ, and IL-10 (in Th2 autoimmune diseases such as systemic lupus erythematosus, SLE, systemic or local sclerosis, SSc, or scleroderma). Relative to healthy individuals, Tregs are partially impaired in autoimmune patients, partly explaining the broken tolerance which characterizes autoimmunity. M1 macrophages induce a strong pro-inflammatory phenotype with the production of cytokines (TNF-α, IL-6, IL-12 and IL-23) and chemokines (CCL-5, CXCL9, CXCL10 and CXCL5), promoting the recruitment of Th1 and Natural killer (NK) cells. The inhibition of pro-inflammatory macrophages can be a strategy of inhibiting inflammation.

Therefore, it is an object of the invention to provide compositions and methods for modulating the immune microenvironment for a desirable immunological outcome.

It is another object of the invention to provide compositions and methods for treating cancer and/or autoimmune diseases.

It is yet another object of the invention to provide compositions and methods for selectively targeting drugs to cells/tissues in need thereof, especially immunosuppressive cells in the tumor microenvironment or pro-inflammatory cells at the site of chronic inflammation associated with autoimmune diseases.

It is a further object to provide compositions and methods for reducing, inhibiting or depleting one or more cells associated with the immunosuppressive tumor microenvironment for enhancing anti-tumor immune response.

It is a further object to provide compositions and methods for reducing, inhibiting or depleting one or more cells associated with the pro-inflammatory microenvironment for ameliorating inflammatory and/or autoimmune diseases.

It is also an object to provide compositions and methods for modulating one or more innate immune sensors, such as the STING pathway, for example, activating or increasing the STING pathway for enhancing anti-tumor immune responses in cancer, or reducing or inhibiting the STING pathway for ameliorating chronic inflammation associated with autoimmune diseases.

### SUMMARY OF THE INVENTION

Compositions and methods for selective delivery of therapeutic agents to tumor-associated immune cells within and surrounding tumors have been developed. The compositions deliver immunotherapeutic agents selectively to the tumor associated macrophage (TAM) cells within the tumor, to create a tumor-suppressive microenvironment and treat the cancer.

Compositions include dendrimers complexed or conjugated with one or more immunomodulatory agents in an amount effective to suppress or inhibit immune cells associated with a tumor in a subject in need thereof. Preferably, the dendrimer is a hydroxyl-terminated dendrimer, most preferably with a majority of the terminal groups being hydroxyl, for example, 25, 50, 60, 75, 80, 90 or 100% of the terminal groups being hydroxyl. In some embodiments, the dendrimer is a generation 4, generation 5, or generation 6 PAMAM dendrimer. Exemplary immunomodulatory agents include STING agonists, CSF1R inhibitors, PARP inhibitors, VEGFR tyrosine kinase inhibitors, MEK inhibitors, TIEII inhibitors, and glutaminase inhibitors, and combinations thereof.

In some embodiments, the immunomodulatory agent is a STING agonist, such as a cyclic dinucleotide GMP-AMP or DMXAA. In other embodiments, the immunomodulatory agent is a CSF1R inhibitor. Exemplary CSF1R inhibitors include PLX3397, PLX108-01, ARRY-382, PLX7486, BLZ945, JNJ-40346527, and GW 2580. In other embodiments, the immunomodulatory agent is a PARP inhibitor, such as Olaparib, Veliparib, Niraparib, or Rucaparib. In other embodiments, the immunomodulatory agent is a VEGFR tyrosine kinase inhibitor. Exemplary VEGFR tyrosine kinase inhibitors include sunitinib, sorafenib, pazopanib, vandetanib, axitinib, cediranib, vatalanib, and motesanib. In other embodiments, the immunomodulatory agent is a MEK inhibitor.

Exemplary MEK inhibitors include Trametinib, Cobimetinib, Binimetinib, Selumetinib, PD-325901, PD035901, and TAK-733. In other embodiments, the immunomodulatory agent is a glutaminase inhibitor. Exemplary glutaminase inhibitors include Bis-2-(5-phenylacetimido-1,2,4-thiadiazol-2-yl)ethyl sulfide (BPTES) and 6-diazo-5-oxo-L-norleucine (DON), azaserine, acivicin, and CB-839. In other embodiments, the immunomodulatory agent is a cytotoxic agent. Exemplary cytotoxic agents include Auristatin E and Mertansine. The immunomodulatory agents can be covalently and/or non-covalently linked to the dendrimer. In some embodiments, the immunomodulatory agent is linked to the dendrimer via a linker or spacer moiety. Exemplary covalent linkages include ether, ester, and amide linkages. For example, in some embodiments, the linker or spacer moiety is bound to the dendrimer via an ether linkage, and/or the linker or spacer moiety is bound to the active agent via an ether, ester, or amide linkage, or combinations thereof. In some embodiments, the dendrimers complexed or conjugated with immunomodulatory agents are complexed or conjugated with one or more additional therapeutic, prophylactic and/or diagnostic agents. Diagnostic or labelling agents can be present in an amount effective to label immune cells associated with a tumor in a subject in need thereof, which may be used for diagnosis, prognosis (such as by assessing metastasis), or to determine efficacy of treatment. Examples of additional therapeutic agents include anti-infectives, anti-inflammatories, and pain alleviating compounds.

Methods of making the dendrimer compositions and pharmaceutical formulations including an effective amount of the dendrimer compositions for administration to a subject in need thereof to reduce inflammation or enhance an anti-tumor response are also provided.

Methods of treating cancer by administering to a subject in need thereof an effective amount of the pharmaceutical compositions to reduce proliferation, metastasis, tumor viability, or to enhance the endogenous anti-tumor response are described. In some embodiments, the methods reduce or inhibit tumor associated macrophages in a subject identified as having cancer. In other embodiments, the methods can enhance tumor-specific cytotoxic T cell responses in a subject identified as having cancer.

Compositions and methods for selective delivery of therapeutic agents to the pro-inflammatory immune cells associated with an inflammatory disease or disorders in a subject in need thereof have also been developed. The compositions deliver immunotherapeutic agents selectively to the pro-inflammatory macrophage (M1 macrophages) cells, to create an anti-inflammatory microenvironment and treat and/or ameliorate one or more symptoms of the diseases. In a particular embodiment, the inflammatory disease is an autoimmune disease.

Compositions including dendrimers complexed or conjugated with one or more immunomodulatory agents in an amount effective to suppress or inhibit pro-inflammatory immune cells associated with a pathological site associated with an autoimmune disease in a subject in need thereof are also described. Preferably, the dendrimer is a hydroxyl-terminated dendrimer, most preferably with a majority of the terminal groups being hydroxyl, for example, 25, 50, 60, 75, 80, 90 or 100% of the terminal groups being hydroxyl. In some embodiments, the dendrimer is a generation 4, generation 5, or generation 6 PAMAM dendrimer. Exemplary immunomodulatory agents include STING antagonists, cytotoxic agents, and combinations thereof. In some embodiments, the immunomodulatory agent is a STING antagonist such as C-178, C-176, C18, Astin C, No₂-cLA, H-151, and alpha-mangostin. The immunomodulatory agents can be covalently and/or non-covalently linked to the dendrimer. In some embodiments, the dendrimers complexed or conjugated with immunomodulatory agents are complexed or conjugated with one or more additional therapeutic, prophylactic and/or diagnostic agents. The diagnostic or labelling agents can be present in an amount effective to label pro-inflammatory immune cells associated with an autoimmune disease in a subject having or suspected of having an autoimmune disease, which may be used for diagnosis, prognosis, or to determine efficacy of treatment. Examples of additional therapeutic agents include anti-infectives, anti-inflammatories, and pain alleviating compounds.

Methods of treating inflammatory diseases and disorders by administering to a subject in need thereof an effective amount of the pharmaceutical compositions are described. In particular embodiments, the inflammatory disease is an autoimmune disease. In some embodiments, the methods reduce or inhibit pro-inflammatory immune cells associated with autoimmune diseases in a subject. In other embodiments, the methods can decrease inflammation associated with autoimmune diseases. In some embodiments, the autoimmune diseases is rheumatoid arthritis, psoriasis, psoriatic arthritis, systemic lupus erythematosus (SLE), type 1 diabetes, inflammatory bowel disease, or thyroid disease. In some embodiments, the inflammatory disease is an inflammatory joint disease, such as osteoarthritis.

Compositions including hydroxyl-terminated dendrimers complexed or conjugated with one or more therapeutic agents in an amount effective for treating one or more disorders of the bone are also described. In preferred embodiments, the dendrimers are covalently conjugated with alendronate. In some embodiments, one or more therapeutic agents are covalently conjugated to the dendrimer via one or more linkers. Methods for treating a disease or disorder of the bone in a subject in need thereof, including administering to the subject a composition including hydroxyl-terminated dendrimers complexed or conjugated with alendronate and one or more therapeutic agents in an amount effective for treating the one or more disorders of the bone, are also described.

### BRIEF DESCRIPTION OF THE DRAWINGS

FIG. 1 is a scheme showing chemical reaction for the synthesis of a dendrimer-DMXAA conjugate.

FIGS. 2A and 2B are schemes showing chemical reaction steps for the synthesis of a dendrimer-GW 2580 ether conjugate (FIG. 2A) and a dendrimer-GW 2580 ester conjugate (FIG. 2B).

FIGS. 3A and 3B are schemes showing chemical reaction steps for the synthesis of a dendrimer-sunitinib conjugate via a hydroxymethyl linkage (FIG. 3A) and an amide linkage (FIG. 3B).

FIGS. 4A and 4B are dot plots showing average radiant efficiency measured by [p/sec/cm²/sr]/[μW/cm²] of tumors in Female C57BL/6 mice 3 days after daily intravenous treatment with PBS (Group 1 ×), and with D-Cy5 (Group 2 ▴); the mean of each group is represented by a horizontal line. FIG. 4C is median average radiance efficiencies plotted on a log scale comparing tumors in mice 3 days after daily intravenous treatment with PBS (Group 1 ×) and with D-Cy5 (Group 2 ▴).

FIG. 5 is a box and whisker plot showing the volume of tumors in Female C57BL/6 mice 3 days after daily intravenous treatment with PBS (Group 1 ×) and with D-Cy5 (Group 2 ▴); with the “box” representing the 25th and 75th percentile of observations, the “line” representing the median of observations, and the “whiskers” representing the extreme observations.

FIGS. 6A-6H are dot plots showing percentage of CD45+ cells in total live cells (FIG. 6A); percentage of conventional CD4+ population in total CD45+ cells (FIG. 6B); percentage of Treg population out of total CD45+ cells (FIG. 6C); percentage of CD8+ population of total CD45+ cells (FIG. 6D); percentage of gMDSC population of total CD45+ cells (FIG. 6E); percentage of M1 macrophage population of total CD45+ cells (FIG. 6F); percentage of M2 macrophage out of total CD45+ cells (FIG. 6G); percentage of mMDSC population of total CD45+ cells (FIG. 6H) in tumors of Female C57BL/6 mice 3 days after daily intravenous treatment with PBS (Group 1 ×) and with D-Cy5 (Group 2 ▴).

FIGS. 7A-7G are dot plots showing percentage of Dendrimer+ cells in total conventional CD4+ population (FIG. 7A); percentage of Dendrimer+ cells in Treg population (FIG. 7B); percentage of Dendrimer+ cells in CD8+ population (FIG. 7C); percentage of Dendrimer+ cells in M1 macrophage population (FIG. 7D); percentage of Dendrimer+ cells in M2 macrophage population (FIG. 7E); percentage of Dendrimer+ cells in gMDSC population (FIG. 7F); percentage of Dendrimer+ cells in mMDSC population (FIG. 7G) in tumors of Female C57BL/6 mice 3 days after daily intravenous treatment with PBS (Group 1 ×) and with D-Cy5 (Group 2 ▴).

FIG. 8 is a line graph showing tumor volume over a treatment period of twenty days in groups treated with vehicle control, sunitinib 60 mg/kg i.p., dendrimer conjugated sunitinib via amide linkage (D-NSA) at 56.7 mg/kg, 11.34 mg/kg, and 2.27 mg/kg; dendrimer conjugated sunitinib via ester linkage (D-CSA) at 57.8 mg/kg, 11.55 mg/kg, and 2.31 mg/kg.

FIG. 9 is a bar graph showing tumor weight in grams at the end of the treatment period in groups treated with vehicle control, sunitinib 60 mg/kg i.p., dendrimer conjugated sunitinib via amide linkage (D-NSA) at 56.7 mg/kg (D-NSA High), 11.34 mg/kg (D-NSA Mid), and 2.27 mg/kg (D-NSA Low); dendrimer conjugated sunitinib via ester linkage (D-CSA) at 57.8 mg/kg (D-CSA High), 11.55 mg/kg (D-CSA Mid), and 2.31 mg/kg (D-CSA Low).

FIG. 10 is a graph showing percentage binding (0-100%) over incubation time (1-5 hr) for hydroxyapatite binding to Alendronate (ALN).

FIGS. 11A-11B are graphs showing paw volume (ml; mean+/−SEM) over time (Days 0-21) for each of 6 groups G1 (CIA, D-CY5); G2 (CIA, ALN D-CY5); G3 (CIA, Vehicle); G4 (Naive, D-CY5); G5 (Naive, ALN D-CY5) and G6 (Naïve, vehicle), in each of left paw (FIG. 11A) and right paw (FIG. 11B), respectively.

FIG. 12 is a bar graph showing contrast index (0-5, mean+/−SEM) for each of 4 groups G1 (CIA, D-CY5); G2 (CIA, ALN D-CY5); G4 (Naive, D-CY5); and G5 (Naive, ALN D-CY5) in hind limb foot of test animals. Contrast index is [Fluor (ROI)−Fluor (ay. ROI autofluorescence)]/[Fluor(ref tissue)−(ay. Ref tissue autofluorescence)].

FIG. 13A is a synthesis scheme of dendrimer conjugated to two different classes of active agents R1 and R2. FIG. 13B shows exemplary R1 groups including capecitabine and gemcitabine, and analogs thereof. FIG. 13C shows exemplary R2 groups such as TIE II inhibitors and analogs thereof.

FIGS. 14A and 14B are synthesis schemes of dendrimer conjugated to two exemplary TLR4 agonists.

FIG. 15 is a synthesis scheme of dendrimer conjugated to an exemplary CSF1R inhibitor.

FIG. 16 is a synthesis scheme for Dendrimer-N-Acetyl-L-cysteine methyl ester conjugate.

### DETAILED DESCRIPTION OF THE INVENTION

### I. Definitions

The terms “active agent” or “biologically active agent” are therapeutic, prophylactic or diagnostic agents used interchangeably to refer to a chemical or biological compound that induces a desired pharmacological and/or physiological effect, which may be prophylactic, therapeutic or diagnostic. These may be a nucleic acid, a nucleic acid analog, a small molecule having a molecular weight less than 2 kD, more typically less than 1 kD, a peptidomimetic, a protein or peptide, carbohydrate or sugar, lipid, or surfactant, or a combination thereof. The terms also encompass pharmaceutically acceptable, pharmacologically active derivatives of active agents, including, but not limited to, salts, esters, amides, prodrugs, active metabolites, and analogs.

The term “prodrug”, refers to a pharmacological substance (drug) that is administered to a subject in an inactive (or significantly less active) form. Once administered, the prodrug is metabolized in the body (in vivo) by enzymatic or chemical reactions, or by a combination of the two, into a compound having the desired pharmacological activity. Prodrugs can be prepared by replacing appropriate functionalities present in the compounds described above with “pro-moieties” as described, for example, in H. Bundgaar, Design of Prodrugs (1985). For further discussion of prodrugs, see, for example, Rautio, J. et al. Nature Reviews Drug Discovery. 7:255-270 (2008).

The term “pharmaceutically acceptable salts” is art-recognized, and includes relatively non-toxic, inorganic and organic acid addition salts of compounds. Examples of pharmaceutically acceptable salts include those derived from mineral acids, such as hydrochloric acid and sulfuric acid, and those derived from organic acids, such as ethanesulfonic acid, benzenesulfonic acid, and p-toluenesulfonic acid. Examples of suitable inorganic bases for the formation of salts include the hydroxides, carbonates, and bicarbonates of ammonia, sodium, lithium, potassium, calcium, magnesium, aluminum, and zinc. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine;

The term “therapeutic agent” refers to an active agent that can be administered to treat one or more symptoms of a disease or disorder.

The term “diagnostic agent”, refers to an active agent that can be administered to reveal, pinpoint, and define the localization of a pathological process. The diagnostic agents can label target cells that allow subsequent detection or imaging of these labeled target cells. In some embodiments, diagnostic agents can, via dendrimer or suitable delivery vehicles, target/bind cancerous cells or cells associated and located at/near tumor site such as tumor associated macrophages.

The term “prophylactic agent”, refers to an active agent that can be administered to prevent disease or to prevent certain conditions, such as a vaccine.

The terms “immunologic”, “immunological” or “immune” response is the development of a beneficial humoral (antibody mediated) and/or a cellular (mediated by antigen-specific T cells or their secretion products) response directed against an immunogen in a recipient patient. Such a response can be an active response induced by administration of immunogen or a passive response induced by administration of antibody or primed T-cells. A cellular immune response is elicited by the presentation of polypeptide epitopes in association with Class I or Class II MHC molecules to activate antigen-specific CD4⁺ T helper cells and/or CD8⁺ cytotoxic T cells. The response may also involve activation of monocytes, macrophages, NK cells, basophils, dendritic cells, astrocytes, microglia cells, eosinophils or other components of innate immunity. The presence of a cell-mediated immunological response can be determined by proliferation assays (CD4⁺ T cells) or CTL (cytotoxic T lymphocyte) assays. The relative contributions of humoral and cellular responses to the protective or therapeutic effect of an immunogen can be distinguished by separately isolating antibodies and T-cells from an immunized syngeneic animal and measuring protective or therapeutic effect in a second subject.

The terms “immunomodulatory agent” or “immunotherapeutic agent” refer to an active agent that can be administered to regulate, enhance, reduce, prolong, decrease or otherwise alter one or more factors of the innate or adaptive immune response in the recipient. Generally, immunomodulatory agents can modulate immune microenvironment for a desired immunological response by targeting one or more immune cells or cell types at a target site, and thus, are not necessarily specific to any cancer type. For example, the blockade of a single molecule, programmed cell-death protein 1 (PD-1) on immune cells, has resulted in anti-tumor activity. In some embodiments, the immunomodulatory agents are specifically delivered to inhibit or reduce suppressive immune cells such as tumor associated macrophages for an enhanced anti-tumor response at a tumor site.

The term “immunosuppressive cells” refer to immune cells that promote tumor growth, angiogenesis, invasion, metastasis, resistance to therapy, or a combination thereof. Exemplary immunosuppressive cells including cancer-associated fibroblasts, myeloid-derived suppressor cells (MDSCs), regulatory T cells (Treg), mesenchymal stromal cells (MSCs) and TIE2-expressing monocytes, and tumor-associated macrophages (TAMs).

The term “pro-inflammatory cells” refer to immune cells that promote pro-inflammatory activities, secretion of pro-inflammatory cytokines such as IL-12, IFN-γ, and TNF-α, or a combination thereof. Exemplary pro-inflammatory cells including pro-inflammatory M1 macrophages or classically activated macrophages (CAMs).

The phrase “pharmaceutically acceptable” or “biocompatible” refers to compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” refers to pharmaceutically acceptable materials, compositions or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient.

The term “therapeutically effective amount” refers to an amount of the therapeutic agent that, when incorporated into and/or onto dendrimers, produces some desired effect at a reasonable benefit/risk ratio applicable to any medical treatment. The effective amount may vary depending on such factors as the disease or condition being treated, the particular targeted constructs being administered, the size of the subject, or the severity of the disease or condition. One of ordinary skill in the art may empirically determine the effective amount of a particular compound without necessitating undue experimentation. In some embodiments, the term “effective amount” refers to an amount of a therapeutic agent or prophylactic agent to reduce or diminish the symptoms of one or more diseases or disorders, such as reducing tumor size (e.g., tumor volume) or reducing or diminishing one or more symptoms of an autoimmune diseases, such as pain and swelling in the wrist and small joints of the hand and feet in patients with rheumatoid arthritis etc. In the case of cancer or tumor, an effective amount of the drug may have the effect of reducing the number of cancer cells; reducing the tumor size; inhibiting cancer cell infiltration into peripheral organs; inhibiting tumor metastasis; inhibiting tumor growth; and/or relieving one or more of the symptoms associated with the disorder. An effective amount can be administered in one or more administrations.

The terms “inhibit” or “reduce” in the context of inhibition, mean to reduce or decrease in activity and quantity. This can be a complete inhibition or reduction in activity or quantity, or a partial inhibition or reduction. Inhibition or reduction can be compared to a control or to a standard level. Inhibition can be 5, 10, 25, 50, 75, 80, 85, 90, 95, 99, or 100%. For example, dendrimer compositions including one or more inhibitors may inhibit or reduce the activity and/or quantity of tumor associated macrophages by about 10%, 20%, 30%, 40%, 50%, 75%, 85%, 90%, 95%, or 99% from the activity and/or quantity of the same cells in equivalent tumor tissues of subjects that did not receive, or were not treated with the dendrimer compositions. In some embodiments, the inhibition and reduction are compared at mRNAs, proteins, cells, tissues and organs levels. For example, an inhibition and reduction in tumor proliferation, or tumor size/volume.

The term “treating” or “preventing” a disease, disorder or condition from occurring in an animal which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected, such as treating the pain of a subject by administration of an analgesic agent even though such agent does not treat the cause of the pain. Desirable effects of treatment include decreasing the rate of disease progression, ameliorating or palliating the disease state, and remission or improved prognosis. For example, an individual is successfully “treated” if one or more symptoms associated with cancer are mitigated or eliminated, including, but are not limited to, reducing the proliferation of cancerous cells, decreasing symptoms resulting from the disease, increasing the quality of life of those suffering from the disease, decreasing the dose of other medications required to treat the disease, delaying the progression of the disease, and/or prolonging survival of individuals.

The phrase “enhancing T-cell function” means to induce, cause or stimulate a T-cell to have a sustained or amplified biological function, or renew or reactivate exhausted or inactive T-cells. Examples of enhancing T-cell function include: increased secretion of Granzyme B, and/or IFN-γ from CD8+ T-cells, increased proliferation, increased antigen responsiveness (e.g., viral, pathogen, or tumor clearance) relative to such levels before the intervention. In one embodiment, the level of enhancement is as least 50%, alternatively 60%, 70%, 80%, 90%, 100%, 120%, 150%, or 200%. The manner of measuring this enhancement is known to one of ordinary skill in the art.

“Tumor immunity” refers to the process in which tumors evade immune recognition and clearance. Thus, as a therapeutic concept, tumor immunity is “treated” when such evasion is attenuated, and the tumors are recognized and attacked by the immune system. Examples of tumor recognition include tumor binding, tumor shrinkage and tumor clearance.

“Immunogenicity” refers to the ability of a particular substance to provoke an immune response. Tumors can be immunogenic and enhancing tumor immunogenicity aids in the clearance of the tumor cells by the immune response.

The term “biodegradable”, generally refers to a material that will degrade or erode under physiologic conditions to smaller units or chemical species that are capable of being metabolized, eliminated, or excreted by the subject. The degradation time is a function of composition and morphology.

The term “dendrimer” includes, but is not limited to, a molecular architecture with an interior core, interior layers (or “generations”) of repeating units regularly attached to this initiator core, and an exterior surface of terminal groups attached to the outermost generation.

The term “functionalize” means to modify a compound or molecule in a manner that results in the attachment of a functional group or moiety. For example, a molecule may be functionalized by the introduction of a molecule which makes the molecule a strong nucleophile or strong electrophile.

The term “targeting moiety” refers to a moiety that localizes to or away from a specific locale. The moiety may be, for example, a protein, nucleic acid, nucleic acid analog, carbohydrate, or small molecule. The entity may be, for example, a therapeutic compound such as a small molecule, or a diagnostic entity such as a detectable label. The locale may be a tissue, a particular cell type, or a subcellular compartment. In one embodiment, the targeting moiety directs the localization of an active agent.

The term “prolonged residence time” refers to an increase in the time required for an agent to be cleared from a patient's body, or organ or tissue of that patient. In certain embodiments, “prolonged residence time” refers to an agent that is cleared with a half-life that is 10%, 20%, 50% or 75% longer than a standard of comparison such as a comparable agent without conjugation to a delivery vehicle such as a dendrimer. In certain embodiments, “prolonged residence time” refers to an agent that is cleared with a half-life of 2, 5, 10, 20, 50, 100, 200, 500, 1000, 2000, 5000, or 10000 times longer than a standard of comparison such as a comparable agent without a dendrimer that specifically target specific cell types associated with tumors.

The terms “incorporated” and “encapsulated” refer to incorporating, formulating, or otherwise including an active agent into and/or onto a composition that allows for release, such as sustained release, of such agent in the desired application. The active agent or other material can be incorporated into a dendrimer, including to one or more surface functional groups of such dendrimer (by covalent, ionic, or other binding interaction), physical admixture, enveloping the agent within the dendritic structure, encapsulated inside the dendritic structure, etc.

The term “neutral surface charge” of a particle refers to the electrokinetic potential (zeta-potential) of a particle that is 0 mV. In some embodiments, the term “near-neutral surface charge” refers to a zeta-potential that is approximately 0 mV, such as from −10 mV to 10 mV, from −5 mV to 5 mV, preferably from −1 mV to 1 mV.

### II. Compositions

Dendrimer complexes suitable for delivering one or more active agent, particularly one or more active agents to prevent, treat or diagnose one or more tumors or autoimmune disease are described.

Compositions of dendrimer complexes including one or more prophylactic, therapeutic, and/or diagnostic agents encapsulated, associated, and/or conjugated in the dendrimers are also provided. Generally, one or more active agent are encapsulated, associated, and/or conjugated in the dendrimer complex at a concentration of about 0.01% to about 30%, preferably about 1% to about 20%, more preferably about 5% to about 20% by weight. In some embodiments, an active agent is covalently conjugated to the dendrimer via one or more linkages such as disulfide, ester, ether, thioester, carbamate, carbonate, hydrazine, and amide, optionally via one or more spacers. In some embodiments, the spacer is an active agent, such as N-acetyl cysteine. Exemplary active agents include anti-inflammatory drugs, chemotherapeutics, anti-seizure agents, vasodilators, and anti-infective agents.

The presence of the additional agents can affect the zeta-potential or the surface charge of the particle. In one embodiment, the zeta potential of the dendrimers is between −100 mV and 100 mV, between −50 mV and 50 mV, between −25 mV and 25 mV, between −20 mV and 20 mV, between −10 mV and 10 mV, between −10 mV and 5 mV, between −5 mV and 5 mV, or between −2 mV and 2 mV. In a preferred embodiment, the surface charge is neutral or near-neutral. The range above is inclusive of all values from −100 mV to 100 mV.

### A. Dendrimers

Dendrimers are three-dimensional, hyperbranched, monodispersed, globular and polyvalent macromolecules having a high density of surface end groups (Tomalia, D. A., et al., Biochemical Society Transactions, 35, 61 (2007); and Sharma, A., et al., ACS Macro Letters, 3, 1079 (2014)). Due to their unique structural and physical features, dendrimers are useful as nano-carriers for various biomedical applications including targeted drug/gene delivery, imaging and diagnosis (Sharma, A., et al., RSC Advances, 4, 19242 (2014); Caminade, A.-M., et al., Journal of Materials Chemistry B, 2, 4055 (2014); Esfand, R., et al., Drug Discovery Today, 6, 427 (2001); and Kannan, R. M., et al., Journal of Internal Medicine, 276, 579 (2014)).

Recent studies have shown that dendrimer surface groups have a significant impact on their biodistribution (Nance, E., et al., Biomaterials, 101, 96 (2016)). Hydroxyl terminated generation 4 PAMAM dendrimers (˜4 nm size) without any targeting ligand cross the impaired BBB upon systemic administration in a rabbit model of cerebral palsy (CP) significantly more (>20 fold) as compared to healthy controls, and selectively target activated microglia and astrocytes (Lesniak, W. G., et al., Mol Pharm, 10 (2013)).

The term “dendrimer” includes, but is not limited to, a molecular architecture with an interior core and layers (or “generations”) of repeating units which are attached to and extend from this interior core, each layer having one or more branching points, and an exterior surface of terminal groups attached to the outermost generation. In some embodiments, dendrimers have regular dendrimeric or “starburst” molecular structures.

Generally, dendrimers have a diameter between about 1 nm to about 50 nm, more preferably between about 1 nm and about 20 nm, between about 1 nm and about 10 nm, or between about 1 nm to about 5 nm. In some embodiments, the diameter is between about 1 nm to about 2 nm. Conjugates are generally in the same size range, although large proteins such as antibodies may increase the size by 5-15 nm. In general, agent is encapsulated in a ratio of agent to dendrimer of between 1:1 to 4:1 for the larger generation dendrimers. In preferred embodiments, the dendrimers have a diameter effective to penetrate tumor tissue and to retain in target cells for a prolonged period of time.

In some embodiments, dendrimers have a molecular weight between about 500 Daltons and about 100,000 Daltons, preferably between about 500 Daltons and about 50,000 Daltons, most preferably between about 1,000 Daltons and about 20,000 Dalton.

Suitable dendrimers scaffolds that can be used include poly(amidoamine), also known as PAMAM, or STARBURST™ dendrimers; polypropylamine (POPAM), polyethylenimine, polylysine, polyester, iptycene, aliphatic poly(ether), and/or aromatic polyether dendrimers. The dendrimers can have carboxylic, amine and/or hydroxyl terminations. In preferred embodiments, the dendrimers have hydroxyl terminations. Each dendrimer of the dendrimer complex may be same or of similar or different chemical nature than the other dendrimers (e.g., the first dendrimer may include a PAMAM dendrimer, while the second dendrimer may be a POPAM dendrimer).

The term “PAMAM dendrimer” means poly(amidoamine) dendrimer, which may contain different cores, with amidoamine building blocks, and can have carboxylic, amine and hydroxyl terminations of any generation including, but not limited to, generation 1 PAMAM dendrimers, generation 2 PAMAM dendrimers, generation 3 PAMAM dendrimers, generation 4 PAMAM dendrimers, generation 5 PAMAM dendrimers, generation 6 PAMAM dendrimers, generation 7 PAMAM dendrimers, generation 8 PAMAM dendrimers, generation 9 PAMAM dendrimers, or generation 10 PAMAM dendrimers. In the preferred embodiment, the dendrimers are soluble in the formulation and are generation (“G”) 4, 5 or 6 dendrimers (i.e., G4-G6 dendrimers), and/or G4-G10 dendrimers, G6-G10 dendrimers, or G2-G10 dendrimers. The dendrimers may have hydroxyl groups attached to their functional surface groups. In preferred embodiments, the dendrimers are generation 4, generation 5, generation 6, generation 7, or generation 8 hydroxyl terminated poly(amidoamine) dendrimers.

Methods for making dendrimers are known to those of skill in the art and generally involve a two-step iterative reaction sequence that produces concentric shells (generations) of dendritic β-alanine units around a central initiator core (e.g., ethylenediamine-cores). Each subsequent growth step represents a new “generation” of polymer with a larger molecular diameter, twice the number of reactive surface sites, and approximately double the molecular weight of the preceding generation. Dendrimer scaffolds suitable for use are commercially available in a variety of generations. Preferable, the dendrimer compositions are based on generation 0, 1, 2, 3, 4, 5, 6, 7, 8, 9, or 10 dendrimeric scaffolds. Such scaffolds have, respectively, 4, 8, 16, 32, 64, 128, 256, 512, 1024, 2048, and 4096 reactive sites. Thus, the dendrimeric compounds based on these scaffolds can have up to the corresponding number of combined targeting moieties, if any, and active agents.

In some embodiments, the dendrimers include a plurality of hydroxyl groups. Some exemplary high-density hydroxyl groups-containing dendrimers include commercially available polyester dendritic polymer such as hyperbranched 2,2-Bis(hydroxyl-methyl)propionic acid polyester polymer (for example, hyperbranched bis-MPA polyester-64-hydroxyl, generation 4), dendritic polyglycerols.

In some embodiments, the high-density hydroxyl groups-containing dendrimers are oligo ethylene glycol (OEG)-like dendrimers. For example, a generation 2 OEG dendrimer (D2-OH-60) can be synthesized using highly efficient, robust and atom economical chemical reactions such as Cu (I) catalyzed alkyne-azide click and photo catalyzed thiol-ene click chemistry. Highly dense polyol dendrimer at very low generation in minimum reaction steps can be achieved by using an orthogonal hypermonomer and hypercore strategy, for example as described in International Patent Publication No. WO 2019094952. In some embodiments, the dendrimer backbone has non-cleavable polyether bonds throughout the structure to avoid the disintegration of dendrimer in vivo, and to allow the elimination of such dendrimers as a single entity from the body (non-biodegradable).

In some embodiments, the dendrimer is able to specifically target a particular tissue region and/or cell type, preferably tumor associated macrophages or pro-inflammatory macrophages involved in autoimmune diseases. In preferred embodiments, the dendrimer is able to specifically target a particular tissue region and/or cell type without a targeting moiety.

In preferred embodiments, the dendrimers have a plurality of hydroxyl (—OH) groups on the surface of the dendrimers. The preferred surface density of hydroxyl (—OH) groups is at least 1 OH group/nm² (number of hydroxyl surface groups/surface area in nm²). For example, in some embodiments, the surface density of hydroxyl groups is more than 2, 3, 4, 5, 6, 7, 8, 9, 10; preferably at least 10, 15, 20, 25, 30, 35, 40, 45, 50, or more than 50 surface groups/surface area in nm². In further embodiments, the surface density of hydroxyl (—OH) groups is between about 1 and about 50, preferably 5-20 OH group/nm² (number of hydroxyl surface groups/surface area in nm²) while having a molecular weight of between about 500 Da and about 10 kDa. In preferred embodiments, the percentage of free, i.e., un-conjugated hydroxyl groups out of total surface groups (conjugated and un-conjugated) on the dendrimer is more than 70%, 75%, 80%, 85%, 90%, 95%, and/or less than 100%. In the case of generation 4 PAMAM dendrimers, the preferred number of free, i.e., un-conjugated hydroxyl groups is more than 50, 51, 52, 53, 54, 55, 56, 57, 58, 59, 60, 61, 62, or 63 out of total 64 surface terminals/groups. In further embodiments, the hydroxyl terminated dendrimers have an effective number of free hydroxyl groups for selective targeting to target cells such as activated microglia, activated microphages, and tumor associated microphages.

In some embodiments, the dendrimers may have a fraction of the hydroxyl groups exposed on the outer surface, with the others in the interior core of the dendrimers. In preferred embodiments, the dendrimers have a volumetric density of hydroxyl (—OH) groups of at least 1 OH group/nm³ (number of hydroxyl groups/volume in nm³). For example, in some embodiments, the volumetric density of hydroxyl groups is 2, 3, 4, 5, 6, 7, 8, 9, 10, or more than 10, 15, 20, 25, 30, 35, 40, 45, and 50 hydroxyl groups/volume in nm³. In some embodiments, the volumetric density of hydroxyl groups is between about 4 to about 50 hydroxyl groups/nm³, preferably between about 5 to about 30 hydroxyl groups/nm³, more preferably between about 10 to about 20 hydroxyl groups/nm³.

B. Coupling Agents and Spacers

Dendrimer complexes can be formed of therapeutically active agents or compounds conjugated or attached to a dendrimer, a dendritic polymer or a hyperbranched polymer. Optionally, the active agents are conjugated to the dendrimers via one or more spacers/linkers via different linkages such as disulfide, ester, ether, carbonate, carbamate, thiol, thioester, hydrazine, hydrazides, N-alkyl, ethyl, and amide linkages. In some embodiments, one or more spacers/linkers between a dendrimer and an agent are designed to provide a releasable or non-releasable form of the dendrimer-active complexes in vivo.

In some embodiments, the attachment occurs via an appropriate spacer that provides an ester bond between the agent and the dendrimer. In some embodiments, the attachment occurs via an appropriate spacer that provides an amide or an ether bond between the agent and the dendrimer. In preferred embodiments, one or more spacers/linkers between a dendrimer and an agent are added to achieve a desired and effective release kinetics in vivo.

The term “spacer” includes moieties and compositions used for linking a therapeutically active agent to the dendrimer. The spacer can be either a single chemical entity or two or more chemical entities linked together to bridge the dendrimer and the active agent. The spacers can include any small chemical entity, peptide or polymers having sulfhydryl, thiopyridine, succinimidyl, maleimide, vinylsulfone, and carbonate terminations.

The spacer can be chosen from among a class of compounds terminating in sulfhydryl, thiopyridine, succinimidyl, maleimide, vinylsulfone and carbonate group. The spacer can include thiopyridine terminated compounds such as dithiodipyridine, N-Succinimidyl 3-(2-pyridyldithio)-propionate (SPDP), Succinimidyl 6-(3-[2-pyridyldithio]-propionamido)hexanoate LC-SPDP or Sulfo-LC-SPDP. The spacer can also include peptides wherein the peptides are linear or cyclic essentially having sulfhydryl groups such as glutathione, homocysteine, cysteine and its derivatives, arg-gly-asp-cys (RGDC), cyclo(Arg-Gly-Asp-d-Phe-Cys) (c(RGDfC)), cyclo(Arg-Gly-Asp-D-Tyr-Cys), and cyclo(Arg-Ala-Asp-d-Tyr-Cys). In some embodiments, the spacer includes a mercapto acid derivative such as 3 mercapto propionic acid, mercapto acetic acid, 4 mercapto butyric acid, thiolan-2-one, 6 mercaptohexanoic acid, 5 mercapto valeric acid and other mercapto derivatives such as 2 mercaptoethanol and 2 mercaptoethylamine. In some embodiments, the spacer includes thiosalicylic acid and its derivatives, (4-succinimidyloxycarbonyl-methyl-alpha-2-pyridylthio)toluene, (3-[2-pyridithio]propionyl hydrazide. In some embodiments, the spacer includes maleimide terminations wherein the spacer includes polymer or small chemical entity such as bis-maleimido diethylene glycol and bis-maleimido triethylene glycol, Bis-Maleimidoethane, and bismaleimidohexane. In some embodiments, the spacer includes vinylsulfone such as 1,6-Hexane-bis-vinylsulfone. In some embodiments, the spacer includes thioglycosides such as thioglucose. In other embodiments, the spacer includes reduced proteins such as bovine serum albumin and human serum albumin, any thiol terminated compound capable of forming disulfide bonds. In particular embodiments, the spacer includes polyethylene glycol having maleimide, succinimidyl and thiol terminations.

The therapeutically active agent, imaging agent, and/or targeting moiety can be either covalently attached or intra-molecularly dispersed or encapsulated. The dendrimer is preferably a PAMAM dendrimer of generation 1 (G1), G2, G3, G4, G5, G6, G7, G8, G9 or G10, having carboxylic, hydroxyl, or amine terminations. In preferred embodiments, the dendrimer is linked to active agents via a spacer ending in ether or amide bonds.

In some embodiments, a non-releasable form of the dendrimer/active agent complex provides enhanced therapeutic efficacy as compared to a releasable form of the same dendrimer/active agent complex. Therefore, in some embodiments, one or more active agent(s) is conjugated to the dendrimer via a spacer that is attached to the dendrimer in a non-releasable manner, for example, by an ether or amide bond. In some embodiments, one or more active agent(s) is attached to the spacer in a non-releasable manner, for example, by an ether or amide bond. Therefore, in some embodiments, one or more active agent(s) is attached to the dendrimer via a spacer that is attached to the dendrimer, and to the active agent(s) in a non-releasable manner. In an exemplary embodiment, one or more active agent(s) is attached to the dendrimer via a spacer that is attached to the dendrimer and the active agent(s) via amide and/or ether bonds. An exemplary spacer is polyethylene glycol (PEG).

1. Dendrimer Conjugation to Active Agents via Ether Linkages

In some embodiments, the compositions include a hydroxyl-terminated dendrimer conjugated to an active agent via an ether linkage, optionally with one or more linkers/spacers are described.

In preferred embodiments, the covalent bonds between the surface groups of the dendrimers and the linkers, or the dendrimers and the active agent (if conjugated without any linking moieties) are stable under in vivo conditions, i.e., minimally cleavable when administered to a subject and/or excreted intact from the body. For example, in preferred embodiments, less than 10%, 5%, 4%, 3%, 2%, 1%, 0.5%, 0.4%, 0.3%, 0.2%, 0.1%, or less than 0.1% of the total dendrimer complexes have active agent cleaved within 24 hours, or 48 hours, or 72 hours after in vivo administration. In one embodiment, the covalent bonds are ether bonds. In further preferred embodiments, the covalent bond between the surface groups of the dendrimers and the linkers, or the dendrimers and the active agent (if conjugated without any linking moieties), are not hydrolytically or enzymatically cleavable bonds, such as ester bonds.

In some embodiments, one or more hydroxyl groups of hydroxyl-terminated dendrimers conjugate to one or more linking moieties and one or more active agents via one or more ether bonds as shown in Formula (I) below.

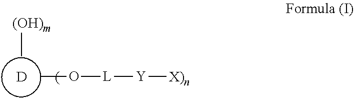

wherein D is a generation 2 to generation 10 poly(amidoamine) (PAMAM) dendrimer; L is one or more linking moieties or spacers; X is an active agent or analog thereof; n is an integer from 1 to 100; and m is an integer from 16 to 4096;
and Y is a linker selected from secondary amides (—CONH—), tertiary amides (—CONR—), sulfonamide (—S(O)₂—NR—), secondary carbamates (—OCONH—; —NHCOO—), tertiary carbamates (—OCONR—; —NRCOO—), carbonate (—O—C(O)—O—), ureas (—NHCONH—; —NRCONH—; —NHCONR—, —NRCONR—), carbinols (—CHOH—, —CROH—), disulfide groups, hydrazones, hydrazides, and ethers (—O—), wherein R is an alkyl group, an aryl group, or a heterocyclic group. Preferably, Y is a bond or linkage that is minimally cleavable in vivo.

In preferred embodiments, Y is a secondary amide (—CONH—).

In one embodiment, L and Y are both absent, and D is directly conjugated to X (an active agent or analog thereof) via an ether linkage.

In one embodiment, D is a generation 4 PAMAM dendrimer; L is one or more linking or spacer moieties; X is a STING agonist, CSF1R inhibitor, PARP inhibitor, VEGFR tyrosine kinase inhibitor, EGFR tyrosine kinase inhibitor, MEK inhibitor glutaminase inhibitors, TIE II antagonist, CXCR2 inhibitor, CD73 inhibitor, arginase inhibitor, PI3K inhibitor, TLR4 agonist, TLR7 agonist, SHP2 inhibitor, STING antagonist, and JAK1 inhibitor, or a derivative, an analogue or a prodrug thereof; n is about 5-15; m is an integer about 49-59; and where n+m=64.

In another embodiment, D is a generation 4 PAMAM dendrimer; L is one or more linking or spacer moieties; X is N, N-didesethyl sunitinib; n is about 5-15; m is an integer about 49-59; and where n+m=64.

In a preferred embodiment, Y is a secondary amide (—CONH—).

In a specific embodiment, the Formula I has the following structure (also referred to as D-4517.2):

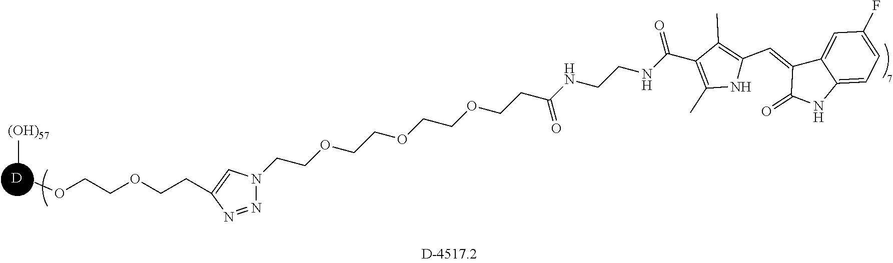

C. Active Agents

Agents to be included in the dendrimer complex to be delivered can be proteins or peptides, sugars or carbohydrate, nucleic acids or oligonucleotides, lipids, small molecules (e.g., molecular weight less than 2000 Dalton, preferably less than 1500 Dalton, more preferably 300-700 Dalton), or combinations thereof. The nucleic acid can be an oligonucleotide encoding a protein, for example, a DNA expression cassette or an mRNA. Representative oligonucleotides include siRNAs, microRNAs, DNA, and RNA. In some embodiments, the active agent is a therapeutic antibody.

Dendrimers have the advantage that multiple therapeutic, prophylactic, and/or diagnostic agents can be delivered with the same dendrimers. In some embodiments, one or more types of active agents are encapsulated, complexed or conjugated to the dendrimer. In particular embodiments, the dendrimers are covalently linked to at least one detectable moiety, in an amount effective to detect a tumor in the subject. In one embodiment, the dendrimer composition has multiple agents, such as a chemotherapeutic agent, immunotherapeutic agent, an anti-seizure agent, a steroid to decrease swelling, an antibiotic, an anti-angiogenic agent, and/or a diagnostic agent, complexed with or conjugated to the dendrimers.

In some embodiments, the dendrimers are complexed with or conjugated to two or more different classes of active agents, providing simultaneous delivery with different or independent release kinetics at the target site. For example, both STING agonists and CSF1R inhibitors are conjugated onto the same dendrimer for delivery to target cells/tissues. In a further embodiment, dendrimer complexes each carrying different classes of active agents are administered simultaneously for a combination treatment. In one embodiment, a generation 4 or generation 6 PAMAM dendrimer is conjugated to sunitinib and a CXCR2 inhibitor, or analogs thereof. In another embodiment, a generation 4 or generation 6 PAMAM dendrimer is conjugated to vincristine and sunitinib, or analogs thereof.

The active agents can also be a pharmaceutically acceptable prodrug of any of the compounds described below. Prodrugs are compounds that, when metabolized in vivo, undergo conversion to compounds having the desired pharmacological activity. Prodrugs can be prepared by replacing appropriate functionalities present in the compounds described above with “pro-moieties” as described, for example, in H. Bundgaar, Design of Prodrugs (1985). Examples of prodrugs include ester, ether or amide derivatives of the compounds described above, polyethylene glycol derivatives of the compounds described above, N-acyl amine derivatives, dihydropyridine pyridine derivatives, amino-containing derivatives conjugated to polypeptides, 2-hydroxybenzamide derivatives, carbamate derivatives, N-oxides derivatives that are biologically reduced to the active amines, and N-mannich base derivatives. For further discussion of prodrugs, see, for example, Rautio, J. et al. Nature Reviews Drug Discovery. 7:255-270 (2008).

1. Immunomodulatory Agents

The dendrimer complexes include one or more therapeutic agents that are immunomodulatory agents. The term “immunomodulatory agent” and “immunotherapeutic agent” mean an active agent that elicits a specific effect upon the immune system of the recipient. Immunomodulation can include suppression, reduction, enhancement, prolonging or stimulation of one or more physiological processes of the innate or adaptive immune response to antigen, as compared to a control. Typically, immunomodulatory agents can modulate immune microenvironment for a desired immunological response (e.g., increasing anti-tumor activity, or increasing anti-inflammatory activities sites in need thereof in autoimmune diseases) by targeting one or more immune cells or cell types at a target site, and thus, are not necessarily specific to any cancer type. In some embodiments, the immunomodulatory agents are specifically delivered to kill, inhibit, or reduce activity or quantity of suppressive immune cells such as tumor-associated macrophages for an enhanced anti-tumor response at a tumor site. In other embodiments, the immunomodulatory agents are specifically delivered to kill, inhibit, or reduce activity or quantity of pro-inflammatory immune cells such as M1 macrophages for reducing pro-inflammatory immune environment at pathogenic sites associated with autoimmune diseases.

Some exemplary immunomodulatory agents used with dendrimers include STING agonists, Colony-Stimulating Factor 1 Receptor (CSF1R) inhibitors, Poly(ADP-ribose) polymerase (PARP) inhibitors, VEGFR tyrosine kinase inhibitors, EGFR tyrosine kinase inhibitors, MEK inhibitors, glutaminase inhibitors, TIE II antagonists, CXCR2 inhibitors, CD73 inhibitors, arginase inhibitors, phosphatidylinositol-3-kinase (PI3K) inhibitors, Toll-like Receptor 4 (TLR4) agonists, TLR7 agonists, and SHP2 (Src homology-2 domain-containing protein tyrosine phosphatase-2) inhibitors. In preferred embodiments, dendrimers associated with or conjugated to one or more of STING agonists, CSF1R inhibitors, PARP inhibitors, VEGFR tyrosine kinase inhibitors, EGFR tyrosine kinase inhibitors, MEK inhibitors, glutaminase inhibitors, TIE II antagonists, CXCR2 inhibitors, CD73 inhibitors, arginase inhibitors, PI3K inhibitors, TLR4 agonists, TLR7 agonists, SHP2 inhibitors, or combinations thereof, are particularly suited for targeting one or more suppressive immune cells in the tumor region as well as reducing the number of cancer cells; reducing the tumor size; inhibiting cancer cell infiltration into peripheral organs; inhibiting tumor metastasis; inhibiting tumor growth; and/or relieving one or more of the symptoms associated with the tumor/cancer. In some embodiments, dendrimers associated with or conjugated to one or more immunomodulatory agents are used in combination with anti-tumor vaccines and/or adoptive cell therapy (ACT) as an adjuvant, for example to increase expression of innate immune genes, infiltration and expansion of activated effector T cells, antigen spreading, and durable immune responses.

In some embodiments, the immunomodulatory agents are any inhibitors targeting one or more of EGFR, ERBB2, VEGFRs, Kit, PDGFRs, ABL, SRC, mTOR, and combinations thereof. In some embodiments, the immunomodulatory agents are one or more inhibitors and analogues thereof, such as crizotinib, ceritinib, alectinib, brigatinib, bosutinib, dasatinib, imatinib, nilotinib, ponatinib, vemurafenib, dabrafenib, ibrutinib, palbociclib, sorafenib, ribociclib, cabozantinib, gefitinib, erlotinib, lapatinib, vandetanib, afatinib, osimertinib, ruxolitinib, tofacitinib, trametinib, axitinib, lenvatinib, nintedanib, pazopanib, regorafenib, sorafenib, sunitinib, vandetanib, bosutinib, dasatinib, dacomitinib, ponatinib, and combinations thereof. In some embodiments, the immunomodulatory agents are tyrosine kinase inhibitors such as HER2 inhibitors, EGFR tyrosine kinase inhibitors. Exemplary EGFR tyrosine kinase inhibitors include gefitinib, erlotinib, afatinib, dacomitinib, and osimertinib.

Additional immunomodulatory agents can include one or more cytotoxic agents that are toxic to one or more immune cells, thus can kill/inhibit one or more types of suppressive immune cells. When delivered selectively to target immune cells such as being conjugated to dendrimers, these agents are able to selectively kill suppressive immune cells or pro-inflammatory immune cells and thus alter immunological microenvironment in and around tumors or in and around pathological sites affected in autoimmune diseases. Cytotoxic immunomodulatory agents include Auristatin E and Mertansine.

STING Agonists In some embodiments, the dendrimers are conjugated or complexed with one or more STING agonists. Stimulator of interferon genes (STING) is a cytosolic receptor that senses both exogenous and endogenous cytosolic cyclic dinucleotides (CDNs), activating TBK1/IRF3 (interferon regulatory factor 3), NF-κB (nuclear factor κB), and STATE (signal transducer and activator of transcription 6) signaling pathways to induce robust type I interferon and proinflammatory cytokine responses. STING is required for the induction of antitumor CD8 T responses in mouse models of cancer. In the tumor microenvironment, T cells, endothelial cells, and fibroblasts, stimulated with STING agonists ex vivo produce type-I IFNs (Corrales, et al., Cell Rep (2015) 11(7):1018-30). By contrast, most studies indicated that tumor cells can inhibit STING pathway activation, potentially leading to immune evasion during carcinogenesis (He, et al., Cancer Lett (2017) 402:203-12; Xia, et al., Cancer Res (2016) 76(22):6747-59). For example, evidence shows that activation of the STING pathway correlates with the induction of a spontaneous antitumor T-cell response involving the expression of type-I IFN genes (Chen, et al., Nat Immunol (2016) 17(10):1142-9; Barber, et al., Nat Rev Immunol (2015) 15(12):760-70; Woo, et al., Immunity (2014) 41(5):830-42). Furthermore, host STING pathway is required for efficient cross-priming of tumor-Ag specific CD8+ T cells mediated by DCs (Woo, et al., Immunity (2014) 41(5):830-42; Deng, et al., Immunity (2014) 41(5):843-52). Based on these results, direct pharmacologic stimulation of the STING pathway has been explored as a cancer therapy.

Additionally, strategies that combine STING immunotherapy with other immunomodulatory agents are being explored. The enforced activation of STING by intratumoral injection of cyclic dinucleotide GMP-AMP (cGAMP), potently enhanced antitumor CD8 T responses leading to growth control of injected and contralateral tumors in mouse models of melanoma and colon cancer. The ability of cGAMP to trigger antitumor immunity was further enhanced when combined with anti-programmed death-1 (PD-1) and anti-cytotoxic T-lymphocyte associated-4 (CTLA-4) antibodies (Demaria, et al., Proc Natl Acad Sci USA (2015) 112(50):15408-13). In other studies, cyclic dinucleotides (CDNs) together with anti-programmed death-L1 blocking antibody incited much stronger antitumor effects than monotherapy in a mouse model of squamous cell carcinoma model as well as of melanoma (Gadkaree, et al., Head Neck (2017) 39(6):1086-94; Wang, et al., Proc Natl Acad Sci USA (2017) 114(7):1637-42). Luo et al. showed encouraging results by combining a STING-activating nanovaccine and an anti-PD1 antibody, which lead to generation of long-term antitumor memory in TC-1 tumor model (Luo, et al., Nat Nanotechnol (2017) 12(7):648-54).

STING agonists can also enhance anti-tumor responses when combined with tumor vaccines. CDNA ligands formulated with granulocyte-macrophage colony-stimulating factor-producing cellular cancer vaccines, termed STINGVAX, showed strong in vivo therapeutic efficacy in several established cancer models (Fu, et al., Sci Transl Med (2015) 7(283):283ra52), and STING agonists in combination with traditional chemotherapeutic agents or radiotherapy can trigger an antitumor response (Xia, et al., Cancer Res (2016) 76(22):6747-59; Baird, et al., Cancer Res (2016) 76(1):50-61).

DMXAA (also known as Vadimezan or ASA404) targets the STING pathway. The antitumor activity of DMXAA has been linked to its ability to induce a variety of cytokines and chemokines, including TNF-α, IP-10, IL-6 and RANTES. DMXAA is also a potent inducer of IFN-β.

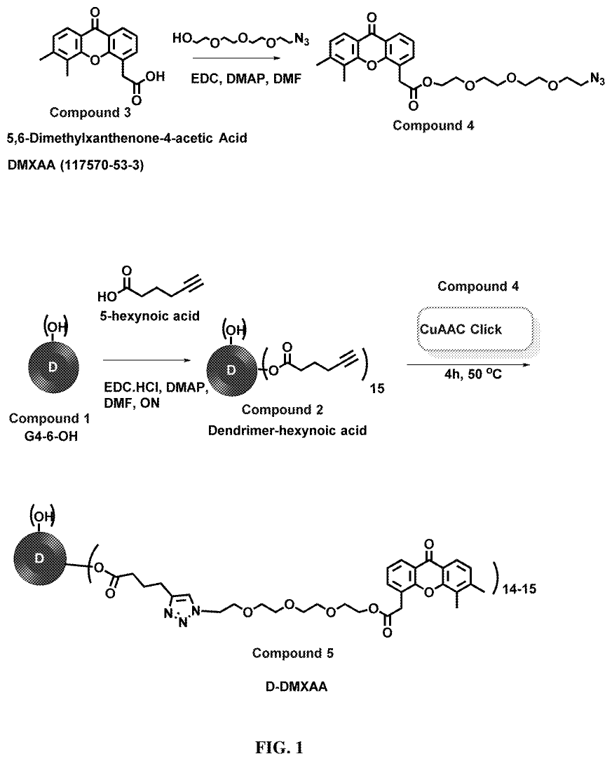

Thus, in some embodiments, the dendrimers are associated with or conjugated to one or more STING agonists or analogues thereof. Exemplary STING agonists include cyclic dinucleotides such as 2′3′ cyclic guanosine monophosphate-adenosine monophosphate (cGAMP) and DMXAA. The STING agonists can be functionalized, for example, with ether, ester, or amide linkage, optionally with one or more spacers/linkers, for ease of conjugation with the dendrimers and/or for desired release kinetics. For example, DMXAA can be modified to DMXAA analogues such as DMXAA ester, DMXAA ether, or DMXAA amide. In preferred embodiments, the STING agonists or derivatives, analogs or prodrugs thereof are conjugated to the dendrimers via Cu (I) catalyzed alkyne-azide click or thiol-ene click chemistry, optionally via one or more spacers/linkers such as polyethylene glycol (PEG). Exemplary conjugation of a STING agonist, e.g., DMXAA to a dendrimer such as a generation 4 or generation 6 PAMAM dendrimer, is shown in FIG. 1.

In preferred cases, the dendrimer complexes including one or more STING agonists are administered in an amount effective to induce/enhance IFN-β production by tumor-infiltrating APCs (e.g., CD11c+CD11b− or CD11c+CD11b+ cells), inhibit tumor growth, reduce tumor size, increase rates of long-term survival, improve response to immune checkpoint blockade, and/or induce immunological memory that protects against tumor re-challenge.

Colony-Stimulating Factor 1 Receptor (CSF1R) Inhibitors

In some embodiments, the dendrimers are conjugated or complexed with one or more CSF1R inhibitors. CSF1R belongs to the type III protein tyrosine kinase receptor family, and binding of CSF1 or the more recently identified ligand, IL-34, induces homodimerization of the receptor and subsequent activation of receptor signaling (Achkova D, Maher J. Biochem Soc Trans. (2016) 44:333-41). CSF1 receptor (CSF1R)-mediated signaling is crucial for the differentiation and survival of the mononuclear phagocyte system and macrophages in particular (Stanley E R, Chitu V. Cold Spring Harb Perspect Biol (2014), 6(6)). As the intratumoral presence of CSF1R+ macrophages correlates with poor survival in various tumor types (Pedersen M B, et al., Histopathology. (2014), 65:490-500; Zhang Q W et al., PLoS One. (2012), 7:e50946), targeting CSF1R signaling in tumor-promoting TAM represents an attractive strategy to eliminate or repolarize these cells. In addition to TAM, CSF1R expression can be detected on other myeloid cells within the tumor microenvironment such as dendritic cells, neutrophils, and myeloid-derived suppressor cells (MDSCs).

A variety of small molecules and monoclonal antibodies (mAbs) directed at CSF1R or its ligand CSF1 are in clinical development both as monotherapy and in combination with standard treatment modalities such as chemotherapy as well as other cancer-immunotherapy approaches. Among the class of small molecules, pexidartinib (PLX3397), an oral tyrosine kinase inhibitor of CSF1R, cKIT, mutant fms-like tyrosine kinase 3 (FLT3), and platelet-derived growth factor receptor (PDGFR)-β, is the subject of the broadest clinical development program in monotherapy, with completed or ongoing studies in c-kit-mutated melanoma, prostate cancer, glioblastoma (GBM), classical Hodgkin lymphoma (cHL), neurofibroma, sarcoma, and leukemia. Additional CSF1R-targeting small molecules, including ARRY-382, PLX7486, BLZ945, and JNJ-40346527, are currently being investigated in solid tumors and cHL. mAbs in clinical development include emactuzumab (RG7155), AMG820, IMC-CS4 (LY3022855), cabiralizumab, MCS110, and PD-0360324, with the latter two being the compounds targeting the ligand CSF1. The phrase “CSF1R inhibitor” is used as a general term for both receptor- and ligand-targeting compounds.

Thus, in some embodiments, the dendrimers are associated with or conjugated to one or more agents for reducing or inhibiting the activities of the CSF1R signaling pathway, such as one or more CSF1R inhibitors or one or more compounds targeting the ligand CSF1. In some embodiments the dendrimers are associated with or conjugated to one or more small molecule CSF1R inhibitors or analogues thereof. Exemplary small molecule CSF1R inhibitors are provided in Current Medicinal Chemistry, 2019, 26, 1-23. Exemplary CSF1R-targeting small molecules include pexidartinib (PLX3397, PLX108-01), ARRY-382, PLX7486, BLZ945, JNJ-40346527, and GW 2580. The small molecule CSF1R inhibitors can be functionalized, for example with ether, ester, or amide linkage, optionally with one or more spacers/linkers, for ease of conjugation with the dendrimers and/or for desired release kinetics. In preferred embodiments, the small molecule CSF1R inhibitors or derivatives, analogs or prodrugs thereof are conjugated to the dendrimers via Cu (I) catalyzed alkyne-azide click or thiol-ene click chemistry, optionally via one or more spacers/linkers such as polyethylene glycol (PEG).

The chemical structures of exemplary CSF1R-targeting small molecules or analogs thereof suitable for conjugation to dendrimers are shown below:

### Structure I: Chemical Structure of CSF1R Inhibitor 1

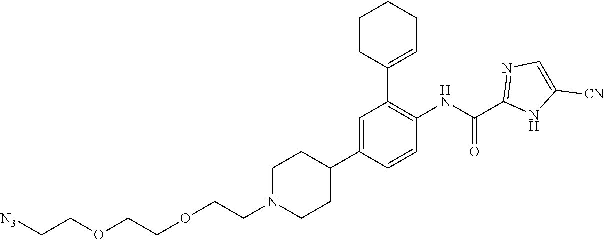 [SMILES: N#Cc1cnc(C(=O)Nc2ccc(C3CCN(CCOCCOCCN=[N+]=[N-])CC3)cc2C2=CCCCC2)[nH]1]

### Structure II: Chemical Structure of CSF1R Inhibitor 2

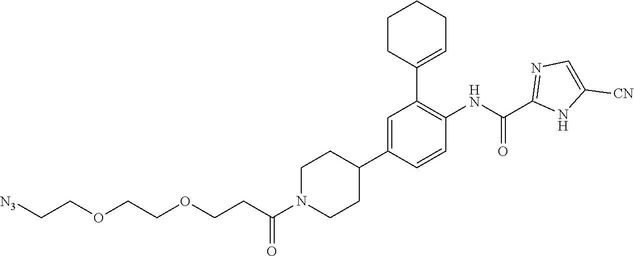 [SMILES: N#Cc1cnc(C(=O)Nc2ccc(C3CCN(C(=O)CCOCCOCCN=[N+]=[N-])CC3)cc2C2=CCCCC2)[nH]1]

### Structure III: Chemical Structure of CSF1R Inhibitor 3

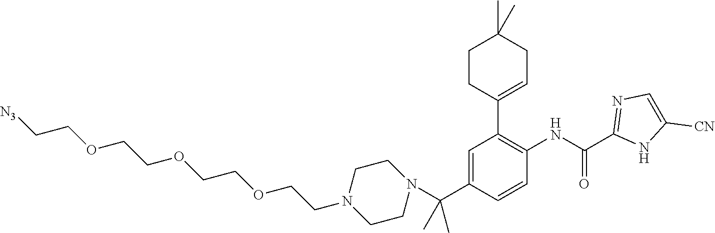 [SMILES: CC1(C)CC=C(c2cc(C(C)(C)N3CCN(CCOCCOCCOCCN=[N+]=[N-])CC3)ccc2NC(=O)c2ncc(C#N)[nH]2)CC1]

### Structure IV: Chemical Structure of CSF1R Inhibitor 4

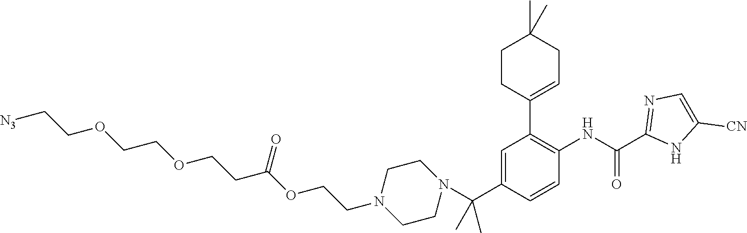 [SMILES: CC1(C)CC=C(c2cc(C(C)(C)N3CCN(CCOC(=O)CCOCCOCCN=[N+]=[N-])CC3)ccc2NC(=O)c2ncc(C#N)[nH]2)CC1]

### Structure V: Chemical Structure of CSF1R Inhibitor 5

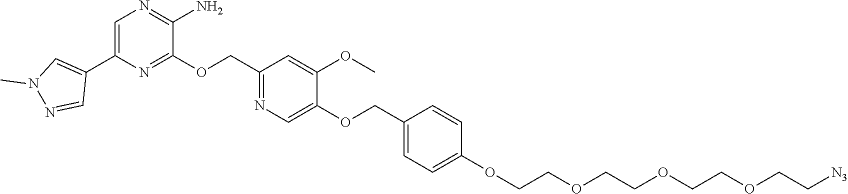 [SMILES: COc1cc(COc2nc(-c3cnn(C)c3)cnc2N)ncc1OCc1ccc(OCCOCCOCCOCCN=[N+]=[N-])cc1]

### Structure VI: Chemical Structure of CSF1R Inhibitor 6

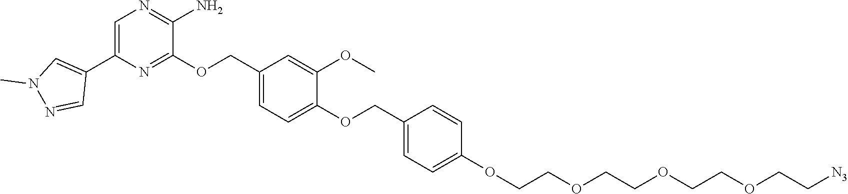 [SMILES: COc1cc(COc2nc(-c3cnn(C)c3)cnc2N)ccc1OCc1ccc(OCCOCCOCCOCCN=[N+]=[N-])cc1]

### Structure VII a-b: Chemical Structure of (a) a CSF1R-E Analog and (b) a Dendrimer-Conjugated CSF1R-E

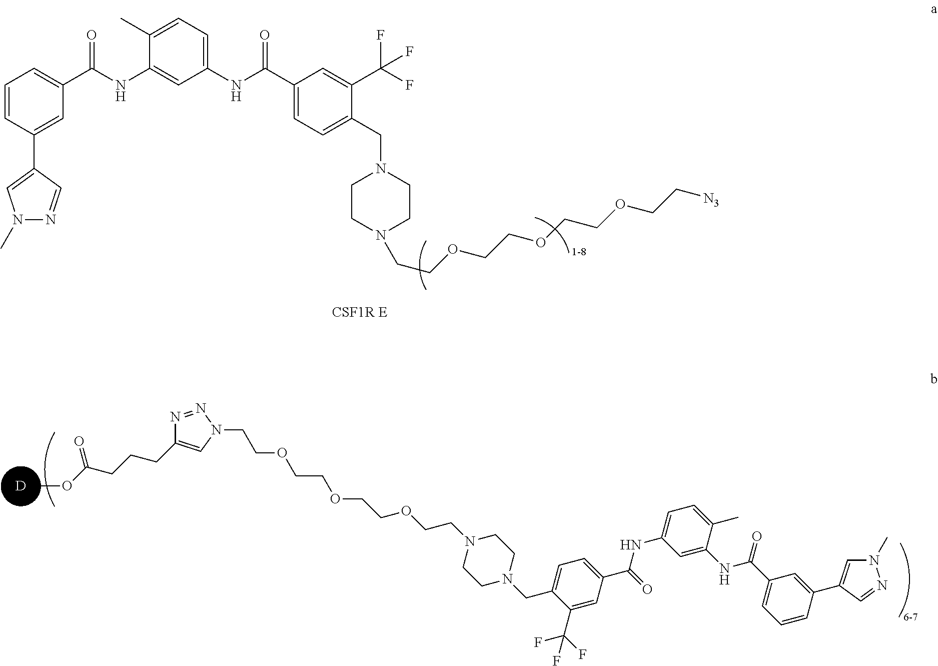

### Structure VIII: Chemical Structure of CSF1R-E Analogue 1

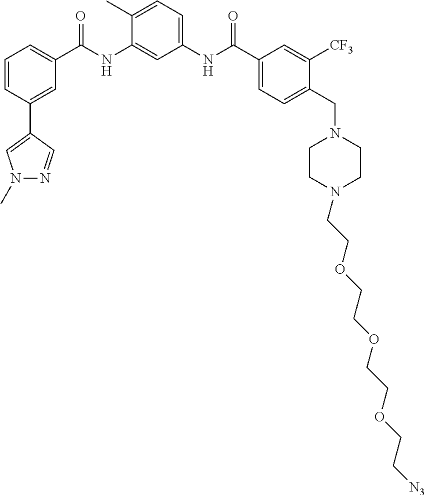 [SMILES: Cc1ccc(NC(=O)c2ccc(CN3CCN(CCOCCOCCOCCN=[N+]=[N-])CC3)c(C(F)(F)F)c2)cc1NC(=O)c1cccc(-c2cnn(C)c2)c1]

The binding affinity of CSF1R-E analogue 1 (Structure VIII) is about 13 nm and the binding affinity of dendrimer conjugated CSF1R-E Analogue 1 (for example, via alkyne-azide click chemistry) is about 200 nm. Thus, in preferred embodiments, the CSF1R inhibitors are conjugated to dendrimers with or without a spacer in such a way that it minimizes the reduction in binding affinity towards CSF1R, for example, less than 1-fold, 2-fold, 3-fold, 4-fold, 5-fold, 10-fold, 20-fold, 30-fold, 40-fold, 50-fold, or 100-fold.

### Structure IX: Chemical Structure of CSF1R Inhibitor F

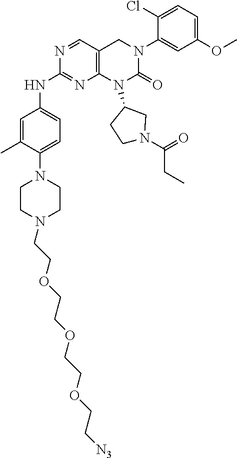

Exemplary CSF1R-targeting mAbs include emactuzumab (RG7155), AMG820, IMC-CS4 (LY3022855), and cabiralizumab. Exemplary mAbs target the ligand CSF1MCS110 and PD-0360324.

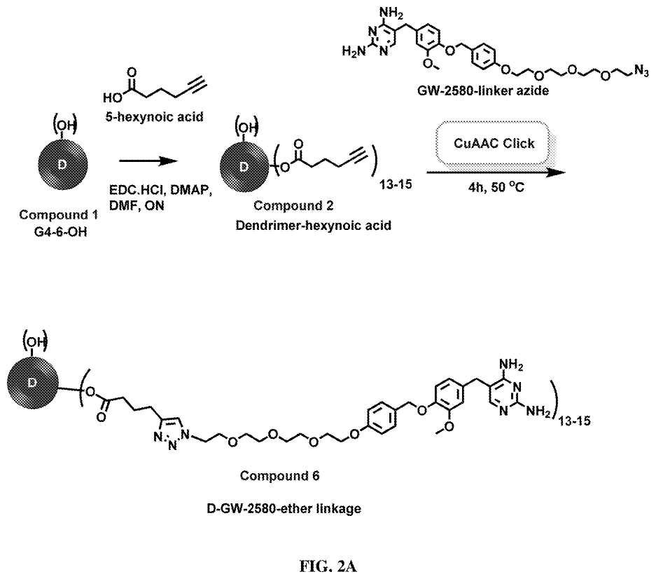

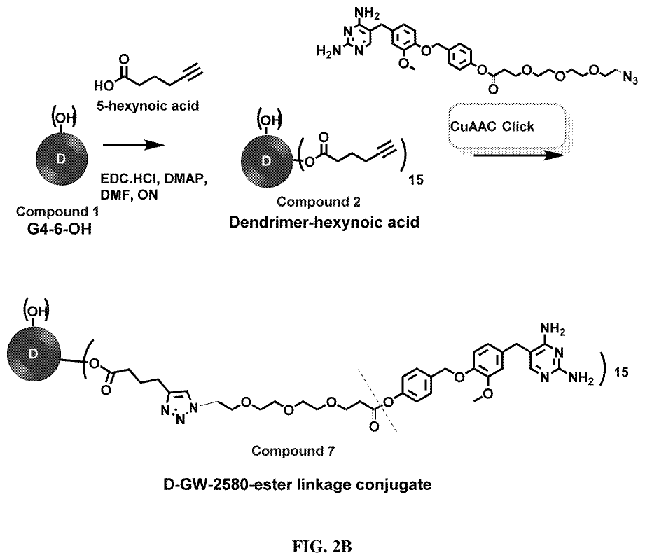

In preferred embodiments, the dendrimers are conjugated to one or more tyrosine kinase inhibitors of CSF1R such as GW2580 (shown as Structure X). The CSF1R inhibitors can be functionalized, for example with ether, ester, or amide linkage, optionally with one or more spacers/linkers, for ease of conjugation with the dendrimers and/or for desired release kinetics. For example, GW2580 can be modified to GW2580 analogues including GW2580 ether, GW2580 ester, and GW2580 amide. In preferred embodiments, the GW2580 or derivatives, analogs or prodrugs thereof are conjugated to the dendrimers via Cu (I) catalyzed alkyne-azide click or thiol-ene click chemistry, optionally via one or more spacers/linkers such as polyethylene glycol (PEG). Exemplary strategies for conjugating a CSF1R inhibitor, e.g., GW2580, to a dendrimer is shown in FIGS. 2A and 2B.

### Structure X: Chemical Structure of GW2580

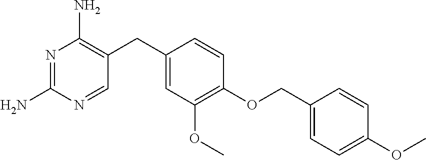 [SMILES: COc1ccc(COc2ccc(Cc3cnc(N)nc3N)cc2OC)cc1]

In one embodiment, the dendrimers are conjugated to a CSF1R inhibitor or an analogue thereof having the following structure.

### Structure XI: Chemical Structure of AR004

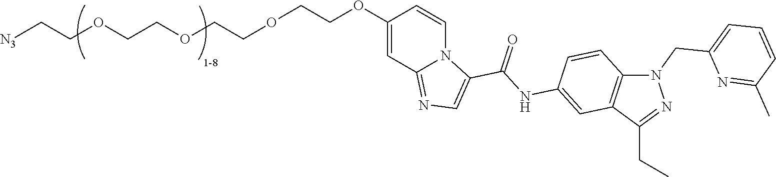

A synthesis route of dendrimers conjugated to AR004 is shown in FIG.

### Poly(ADP-Ribose) Polymerase (PARP) Inhibitors

In some embodiments, dendrimers are conjugated or complexed with one or more PARP inhibitors. Poly(ADP-ribose) polymerases (PARPs) are a family of 17 nucleoproteins characterized by a common catalytic site that transfers an ADP-ribose group on a specific acceptor protein using NAD+ as cofactor. Poly(ADP-ribose) polymerase (PARP) inhibitors

Olaparib (C₂₄H₂₃FN₄O₃) was the first PARP inhibitor introduced in clinical practice. Niraparib is a potent and selective inhibitor of PARP-1 and PARP-2. Rucaparib is a potent PARP inhibitor, approved by FDA in December 2016 and by EMA in May 2018 for the treatment, as single agent, of HGSOC patients with gBRCAm or sBRCAm, relapsed after at least two chemotherapy lines.

In some embodiments, dendrimer complexes include one or more PARP inhibitors such as olaparib, niraparib, and rucaparib. The PARP inhibitors can be functionalized, for example with ether, ester, or amide linkage, optionally with one or more spacers/linkers, for ease of conjugation with the dendrimers and/or for desired release kinetics. In preferred embodiments, the PARP inhibitors or derivatives, analogs or prodrugs thereof are conjugated to the dendrimers via Cu (I) catalyzed alkyne-azide click or thiol-ene click chemistry, optionally via one or more spacers/linkers such as polyethylene glycol (PEG).

### VEGFR Tyrosine Kinase Inhibitor

In some embodiments, dendrimers are conjugated to one or more VEGF Tyrosine Kinase inhibitors. Tyrosine kinases are important cellular signaling proteins that have a variety of biological activities including cell proliferation and migration. Multiple kinases are involved in angiogenesis, including receptor tyrosine kinases such as the vascular endothelial growth factor receptor (VEGFR). Anti-angiogenic tyrosine kinase inhibitors in clinical development primarily target VEGFR-1, -2, -3, epidermal growth factor receptor (EGFR), platelet-derived growth factor receptor (PDGFR), PDGFR-β, KIT, fms-related tyrosine kinase 3 (FLT3), colony stimulating factor-1 receptor (CSF-1R), Raf, and RET.

The VEGFR family includes three related receptor tyrosine kinases, known as VEGFR-1, -2, and -3, which mediate the angiogenic effect of VEGF ligands (Hicklin D J, Ellis L M. J Clin Oncol. (2005), 23(5):1011-27). The VEGF family encoded in the mammalian genome includes five members: VEGF-A, VEGF-B, VEGF-C, VEGF-D, and placental growth factor (PlGF). VEGFs are important stimulators of proliferation and migration of endothelial cells. VEGF-A (commonly referred to as VEGF) is the major mediator of tumor angiogenesis and signals through VEGFR-2, the major VEGF signaling receptor (Kerbel R S, N Engl J Med. (2008), 358(19):2039-49).

Most notable angiogenesis inhibitors target the vascular endothelial growth factor signaling pathway, such as the monoclonal antibody bevacizumab (Avastin, Genentech/Roche) and two kinase inhibitors sunitinib (SU11248, Sutent, Pfizer) and sorafenib (BAY43-9006, Nexavar, Bayer). Bevacizumab was the first angiogenesis inhibitor that was clinically approved, initially for treatment of colorectal cancer and recently also for breast cancer and lung cancer. The small-molecule tyrosine kinase inhibitors sunitinib and sorafenib target the VEGF receptor (VEGFR), primarily VEGFR-2, and have shown clinical efficacy in diverse cancer types. Both drugs have shown benefit in patients with renal cell cancer (Motzer R J, Bukowski R M, J Clin Oncol. (2006); 24(35):5601-8). In addition, sunitinib has been approved for treatment of gastrointestinal stromal tumors (GISTs). Sorafenib inhibits Raf serine kinase as well and has been approved for treatment of hepatocellular cancer as well. Cediranib is an oral tyrosine kinase inhibitor of VEGF receptor (VEGFR).

In some embodiments, dendrimers are conjugated to one or more VEGF receptor inhibitors including Sunitinib (SU11248; SUTENT®), Sorafenib (BAY439006; NEXAVAR®), Pazopanib (GW786034; VOTRIENT®), Vandetanib (ZD6474; ZACTIMA®), Axitinib (AG013736), Cediranib (AZD2171; RECENTIN®), Vatalanib (PTK787; ZK222584), Dasatinib, Nintedanib, and Motesanib (AMG706), or analogues thereof.

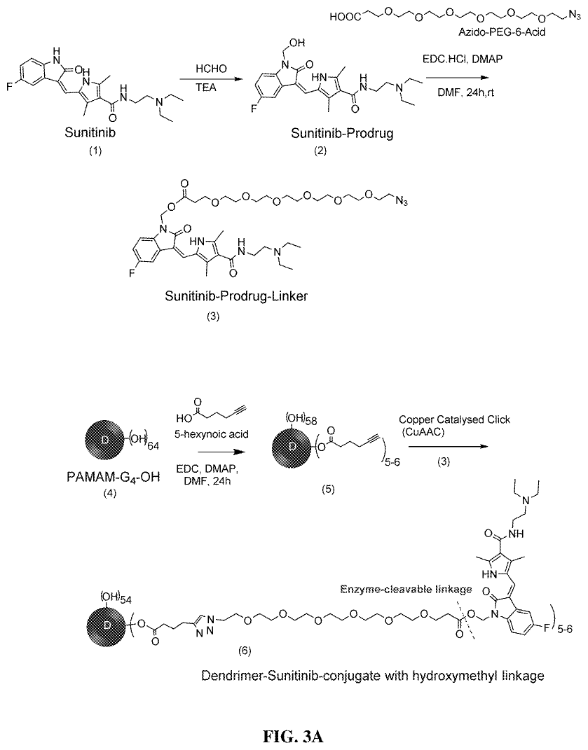

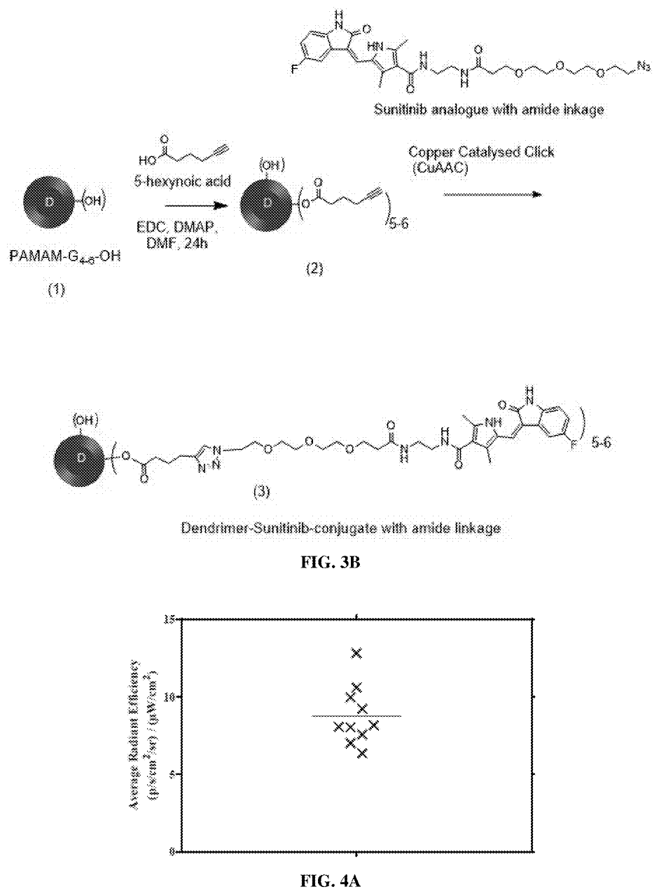

In some embodiments, the VEGF receptor inhibitors can be functionalized with one or more spacers/linkers, for example with ether, ester, or amide linkage, optionally with one or more spacers/linkers, for ease of conjugation with the dendrimers and/or for desired release kinetics. In preferred embodiments, the one or more VEGF receptor inhibitors or derivatives, analogs or prodrugs thereof are conjugated to the dendrimers via Cu (I) catalyzed alkyne-azide click or thiol-ene click chemistry, optionally via one or more spacers/linkers such as polyethylene glycol (PEG). For example, sunitinib can be modified to sunitinib with an ester linkage, or with an amide linkage (FIGS. 3A and 3B). Exemplary conjugation of a VEGF receptor inhibitor, e.g., sunitinib to a dendrimer is shown in FIGS. 3A (via a hydroxymethyl linkage) and 3B (via an amide linkage). In one embodiment, the sunitinib analog is N, N-didesethyl sunitinib.

Exemplary VEGF receptor inhibitor analogues with a functional spacer/linkage are shown below in Structure XII, Structure XIII and Structure XIV.

### Structure XII a-b: Chemical Structures of Sorafenib Analogues

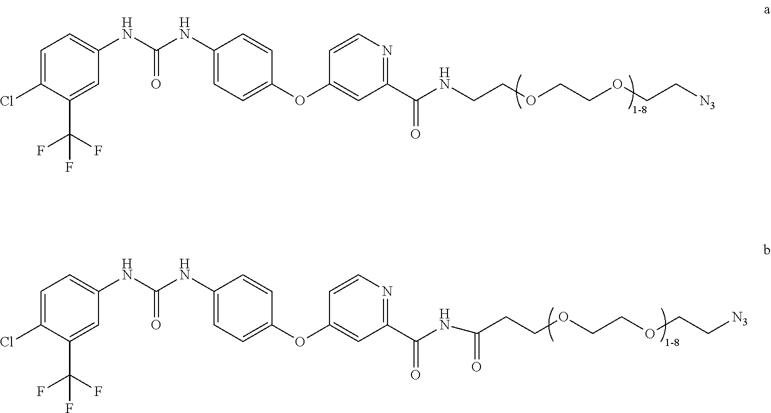

### Structure XIII a-d: Chemical Structures of Nintedanib and Analogues

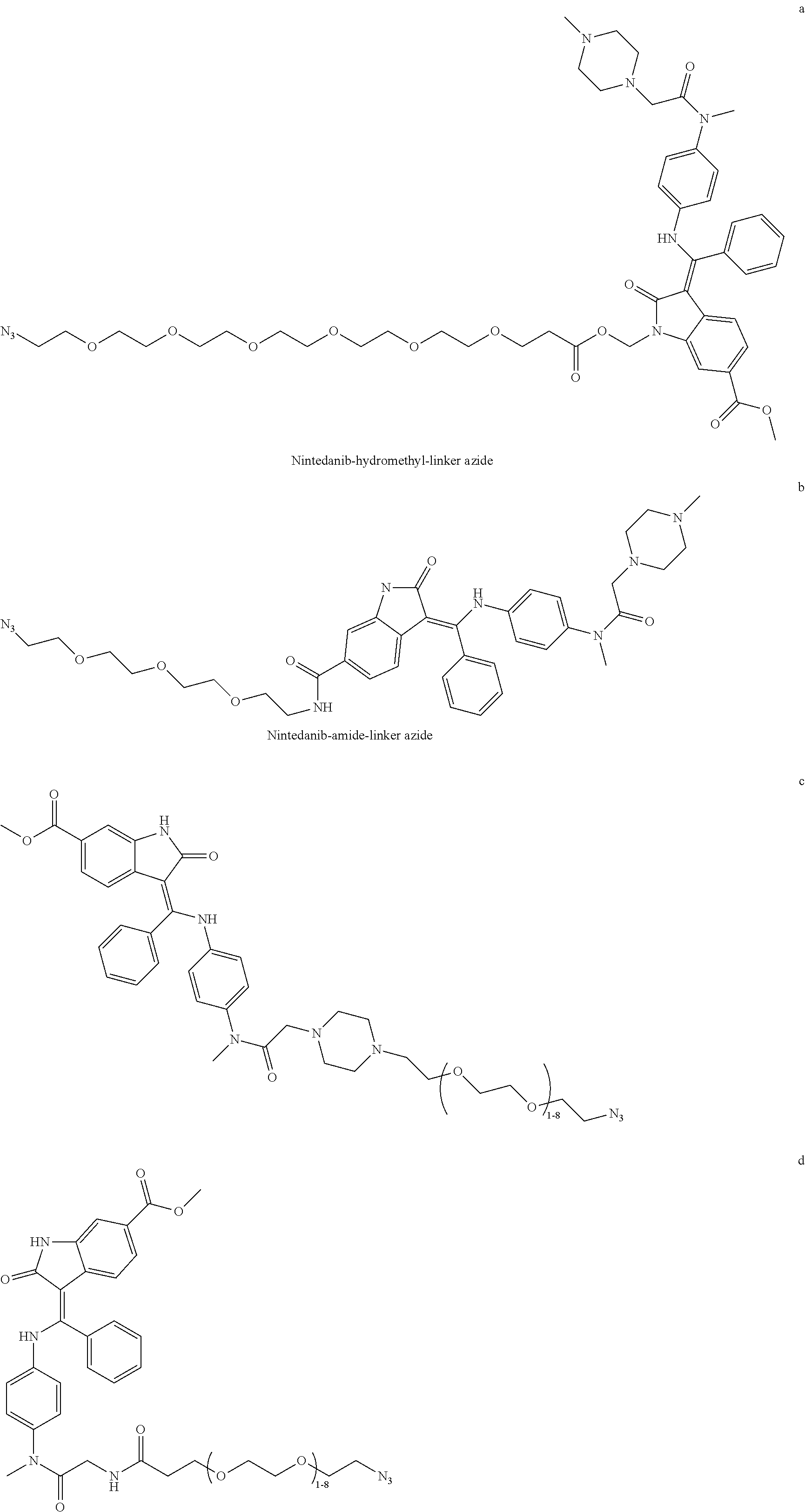

### Structure XIV: Chemical Structures of Orantinib Analogues

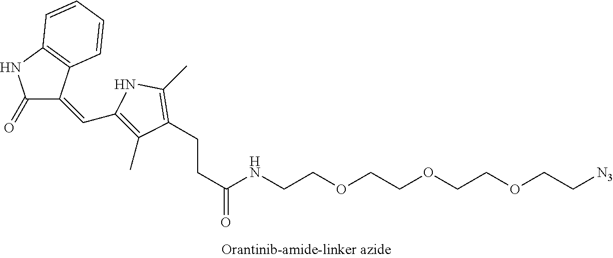 [SMILES: Cc1[nH]c(/C=C2/C(=O)Nc3ccccc32)c(C)c1CCC(=O)NCCOCCOCCOCCN=[N+]=[N-]]

MEK Inhibitors

In some embodiments, dendrimers are conjugated or complexed with one or more MEK inhibitors. The mitogen-activated protein kinase (MAPK) cascade is a critical pathway for human cancer cell survival, dissemination, and resistance to drug therapy. The MAPK/ERK (extracellular signal regulated kinases) pathway is a convergent signaling node receiving input from numerous stimuli, including internal metabolic stress and DNA damage pathways, and altered protein concentrations, as well as via signaling from external growth factors, cell-matrix interactions, and communication from other cells.

In some embodiments, dendrimers are conjugated to one or more MEK inhibitors, such as Refametinib, Pimasertib, Trametinib (GSK1120212), Cobimetinib (or XL518), Binimetinib (MEK162), Selumetinib, CI-1040 (PD-184352), PD325901, PD035901, PD032901, and TAK-733, or analogues thereof. In preferred embodiments, the MEK inhibitors are functionalized, for example with ether, ester, or amide linkage, optionally with one or more spacers/linkers, for ease of conjugation with the dendrimers and/or for desired release kinetics. In preferred embodiments, the MEK inhibitors or derivatives, analogs or prodrugs thereof are conjugated to the dendrimers via Cu (I) catalyzed alkyne-azide click or thiol-ene click chemistry, optionally via one or more spacers/linkers such as polyethylene glycol (PEG). For example, binimetinib can be modified to binimetinib ester, binimetinib ether, or binimetinib amide; trametinib can be modified to trametinib ether, trametinib ester, or trametinib amide; pimasertib can be modified to pimasertib ester and pimasertib ether etc. Exemplary MEK inhibitors and their analogus thereof are shown below: binimetinib functionalized with a PEG linker and an azide group via an ester linkage (Structure XV) and via an ether linkage (Structure XVI); trametinib analogue functionalized with a PEG linker and an azide group via an amide linkage (Structure XVII); and pimasertib analogue functionalized with a PEG linker and an azide group via an ester linkage (Structure XVIII).

### Structure XV: Chemical Structure of Binimetinib Analogue 1

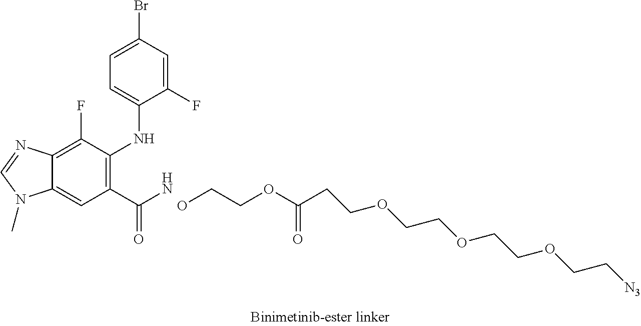

### Structure XVI: Chemical Structure of Binimetinib Analogue 2

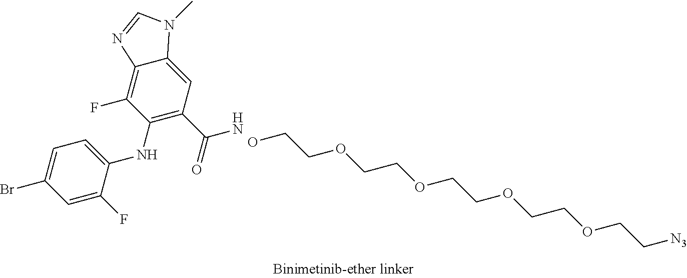

### Structure XVII: Chemical Structure of Trametinib Analogue

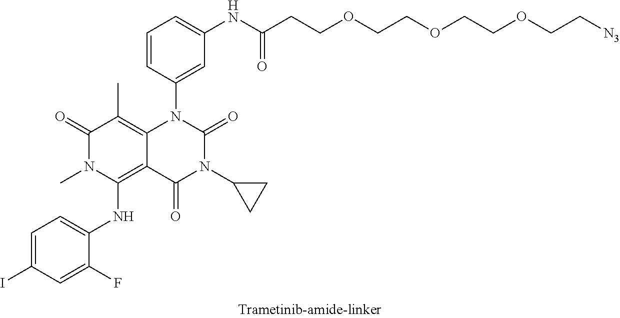 [SMILES: Cc1c(=O)n(C)c(Nc2ccc(I)cc2F)c2c(=O)n(C3CC3)c(=O)n(-c3cccc(NC(=O)CCOCCOCCOCCN=[N+]=[N-])c3)c12]

### Structure XVIII: Chemical Structure of Pimasertib Analogue

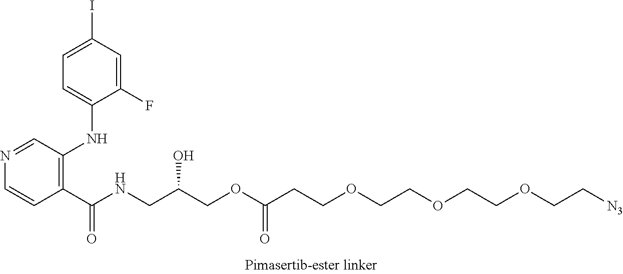 [SMILES: [N-]=[N+]=NCCOCCOCCOCCC(=O)OC[C@@H](O)CNC(=O)c1ccncc1Nc1ccc(I)cc1F]

Glutaminase Inhibitors

In some embodiments, dendrimers are conjugated or complexed with one or more glutaminase inhibitors. Glutaminase (GLS), which is responsible for the conversion of glutamine to glutamate, plays a vital role in up-regulating cell metabolism for tumor cell growth. Exemplary glutaminase inhibitors include Bis-2-(5-phenylacetimido-1,2,4-thiadiazol-2-yl)ethyl sulfide (BPTES), 6-diazo-5-oxo-L-norleucine (DON), azaserine, acivicin, and CB-839. In some embodiments, the glutaminase inhibitors are BPTES analogs such as JHU-198, JHU-212, and JHU-329 (Thomas A G et al., Biochem Biophys Res Commun. (2014); 443(1): 32-36).

In some embodiments, dendrimers are conjugated to one or more glutaminase inhibitors, such as BPTES, DON, azaserine, acivicin, CB-839, JHU-198, JHU-212, and JHU-329. The glutaminase inhibitors can be functionalized, for example with ether, ester, or amide linkage, optionally with one or more spacers/linkers, for ease of conjugation with the dendrimers and/or for desired release kinetics. In preferred embodiments, the glutaminase inhibitors or derivatives, analogs or prodrugs thereof, are conjugated to the dendrimers via Cu (I) catalyzed alkyne-azide click or thiol-ene click chemistry, optionally via one or more spacers/linkers such as polyethylene glycol (PEG). In preferred embodiments, dendrimers are conjugated to CB-839, or a derivative, analog or prodrug, or a pharmacologically active salt thereof. CB-839 has the following structure:

### Structure XIX: Chemical Structure of CB-839

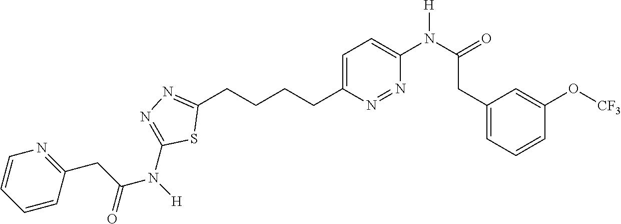 [SMILES: O=C(Cc1cccc(OC(F)(F)F)c1)Nc1ccc(CCCCc2nnc(NC(=O)Cc3ccccn3)s2)nn1]

In some embodiments, dendrimers are conjugated to glutamine analog or antagonist L-[αS,5S]-α-amino-3-chloro-4,5-dihydro-5-isoxazoleacetic acid (acivicin), or a derivative, analog or prodrug, or a pharmacologically active salt thereof. Chemical structure of Acivicin is shown below in Structure XX.

### Structure XX:

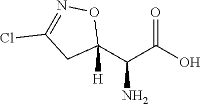 [SMILES: N[C@H](C(=O)O)[C@@H]1CC(Cl)=NO1]

Acivicin has been the subject of clinical trials for the treatment of cancer. Dosages and formulations are known in the art, see, for example, Hidalgo, Clinical Cancer Research, 4(11): 2763-2770 (1998), U.S. Pat. Nos. 3,856,807, 3,878,047, and 5,087,639. In one embodiment, dendrimers are conjugated to acivicin. In preferred embodiments, acivicin is functionalized, for example with ether, ester, N-alkyl, or amide linkage, optionally with one or more spacers/linkers such as polyethylene glycol (PEG), prior to conjugation to dendrimers.

TIE II Antagonists

In some embodiments, the dendrimers are complexed with or conjugated to one or more TIE II antagonists. Angiopoietin-1 receptor also known as CD202B (cluster of differentiation 202B), or TIE II, is a protein that in humans is encoded by the TEK gene. It is an angiopoietin receptor. The angiopoietins are protein growth factors required for the formation of blood vessels (angiogenesis), which supports tumor growth and development. Therefore, in some embodiments, dendrimers are conjugated to one or more TIE II antagonists.

The TIE II antagonists can be functionalized, for example, with ether, ester, or amide linkage, optionally with one or more spacers/linkers, for ease of conjugation with the dendrimers and/or for desired release kinetics. The chemical structure of an exemplary TIE II inhibitor is shown below as Structure XXI. TIE II inhibition of the free TIE II antagonist has a dissociation constant, K_{d}, about 8.8 nm and the TIE II inhibition of dendrimer conjugated TIE II antagonist (Structure XXI) has a dissociation constant, K_{d}, about 25 nm. Thus, in preferred embodiments, TIE II antagonists are conjugated to dendrimers with or without a spacer in such a way that it minimizes the reduction in TIE II inhibition, for example, less than 1-fold, 2-fold, 3-fold, 4-fold, 5-fold, 10-fold, 20-fold, 30-fold, 40-fold, 50-fold, and 100-fold.

### Structure XXI: TIE II Antagonist 1

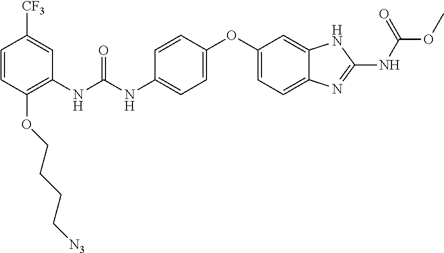 [SMILES: COC(=O)Nc1nc2ccc(Oc3ccc(NC(=O)Nc4cc(C(F)(F)F)ccc4OCCCCN=[N+]=[N-])cc3)cc2[nH]1]

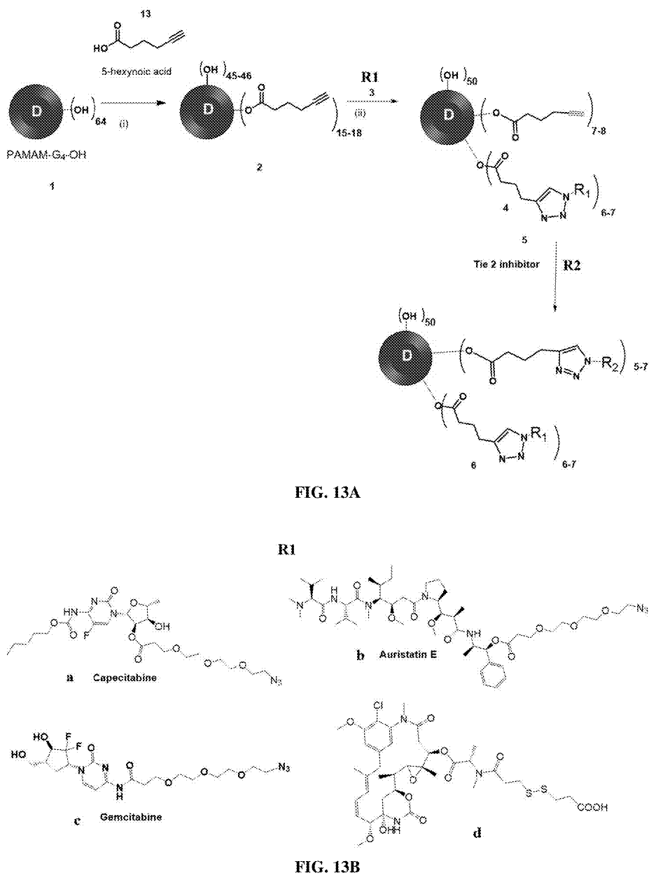

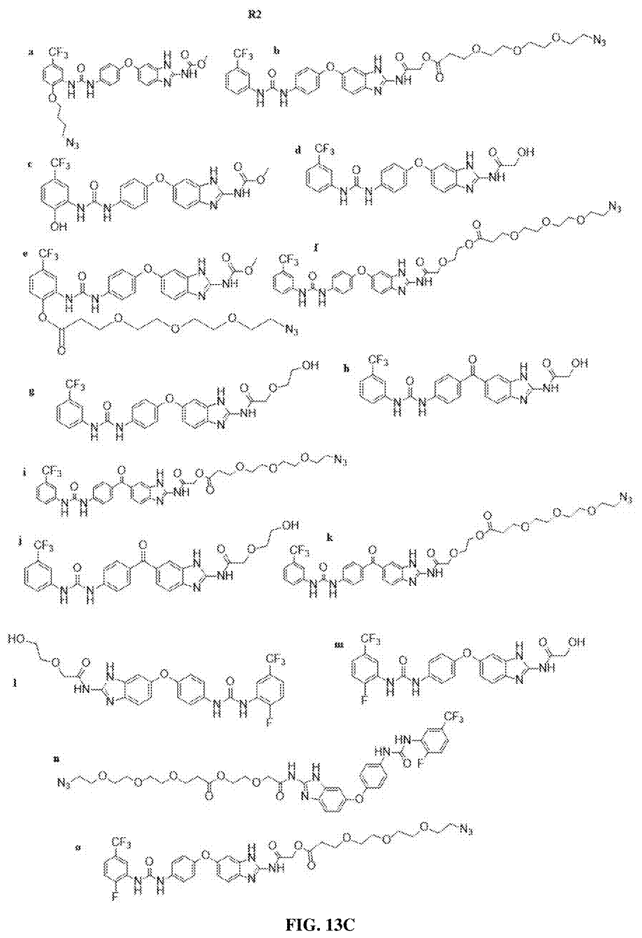

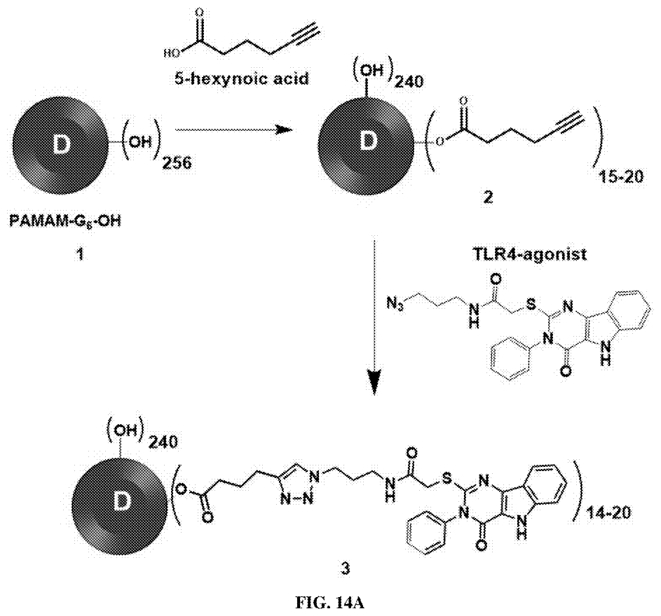

In some embodiments, the dendrimers are complexed with or conjugated to two or more different classes of active agents, providing simultaneous delivery with different or independent release kinetics at the target site. In one embodiment, a generation 4 or generation 6 PAMAM dendrimer is conjugated to a TIE II inhibitor and gemcitabine, or analogs thereof. In another embodiment, a generation 4 or generation 6 PAMAM dendrimer is conjugated to a TIE II inhibitor and capecitabine, or analogs thereof. Exemplary synthesis routes of dendrimers conjugated to two or more different classes of active agents are shown in FIGS. 13A-13C.

CXCR2 Inhibitors

In some embodiments, dendrimers are associated with or conjugated to one or more CXCR2 inhibitors. CXCR2 is expressed by many tumor cells and is involved in the chemotherapy resistance in different preclinical models of cancer (Poeta V M et al., Front Immunol. 2019; 10: 379). In breast cancer cells, CXCR2 deletion resulted in better response to Paclitaxel. In a melanoma model, the CXCR2 inhibitor Navarixin synergized with MEK inhibition whereas, in an ovarian tumor model, the CXCR2 inhibitor SB225002 improved the antiangiogenic therapy Sorafenib. In human gastric cancer, Reparixin, a CXCR1 and CXCR2 inhibitor, enhanced the efficacy of 5-fluorouracil.

CXCR2 targeting also inhibits tumor growth because it affects myeloid cell infiltration. In pancreatic tumors, CXCR2 inhibition prevented the accumulation of neutrophils unleashing the T cell response, resulting in inhibition of metastatic spreading and improved response to anti-PD-1. Interestingly, the combined treatment of CXCR2 and CCR2 inhibitors limited the compensatory response of TAMs, increased antitumor immunity and improved response to FX. Finally, in a prostate cancer model, CXCR2 inhibition by SB265610, decreased recruitment of myeloid cells and enhanced Docetaxel-induced senescence, limiting tumor growth.

Thus, in some embodiments, dendrimers are associated with or conjugated to one or more CXCR2 inhibitors such as Navarixin, SB225002, SB332235, SB265610, Reparixin, and AZD5069. In preferred embodiments, dendrimers are conjugated to Navarixin, SB225002, or SB332235, or a derivative, analog or prodrug, or a pharmacologically active salt thereof. The CXCR2 inhibitors can be functionalized, for example with ether, ester, N-alkyl, or amide linkage, for ease of conjugation with the dendrimers and/or for desired release kinetics. In some embodiments, the CXCR2 inhibitors are conjugated to the dendrimers via N-alkyl linkage using click chemistry.

CD73 Inhibitors

In some embodiments, dendrimers are conjugated to or complexed with one or more CD73 inhibitors. CD73 converts extracellular adenosine monophosphate (AMP) into immunosuppressive adenosine, which shuts down anti-tumor immune surveillance at the level of T cells and natural killer (NK) cells, dendritic cells (DCs), myeloid-derived suppressor cells (MDSCs), and tumor associated macrophages (TAMs). In cancer, upregulation of CD73 expression in tumor cells and cells in the tumor stroma results in an increase in adenosine production, which leads to inhibition of T cell and NK cell cytotoxicity, cytokine production and proliferation as well as suppression of antigen-presenting cells (APCs); enhanced regulatory T cell (Treg) proliferation and suppressive activity, and MDSCs and macrophage M2 polarization. These changes enable tumor growth and disease progression.

Thus, in some embodiments, dendrimers are conjugated to one or more CD73 inhibitors such as non-hydrolyzable AMP analogs such as adenosine 5′-(α,β-methylene)diphosphate (APCP)), flavonoid-based compounds such as quercetin, and purine nucleotide analogs such as tenofovir and sulfonic acid compounds. In preferred embodiments, dendrimers are conjugated to one or more CD73 inhibitors including APCP, quercetin, or tenofovir, or a derivative, analog or prodrug, or a pharmacologically active salt thereof. The CD73 inhibitors can be functionalized, for example with ether, ester, or amide linkage, optionally with one or more spacers/linkers, for ease of conjugation with the dendrimers and/or for desired release kinetics. In preferred embodiments, the CD73 inhibitors or derivatives, analogs or prodrugs thereof, are conjugated to the dendrimers via Cu (I) catalyzed alkyne-azide click or thiol-ene click chemistry.

In some embodiments, one or more CD73 inhibitors and/or derivatives or analogs thereof having structures as shown in Structure XXII a-i and Structure XXIII a-c below are suitable for conjugation to dendrimers.

### Structure XXII a-i: Structures of CD73 Inhibitors and Analogs Thereof

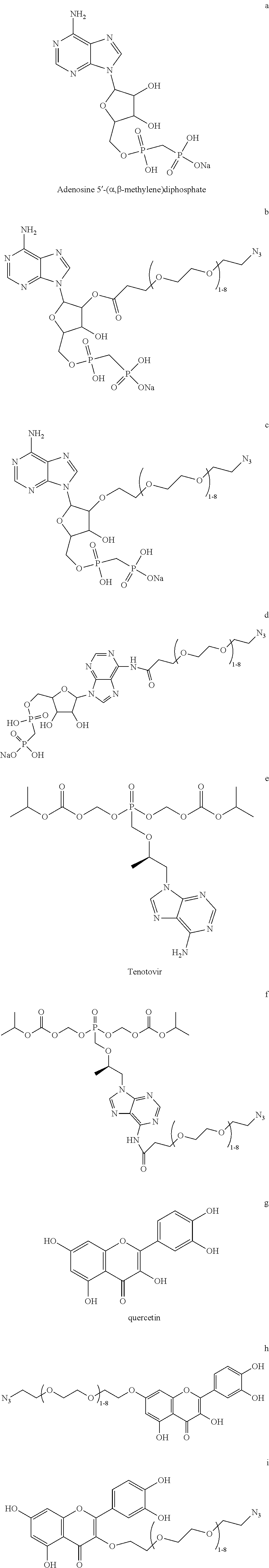

### Structure XXIII a-c: Structures of CD73 Inhibitors and Analogs Thereof

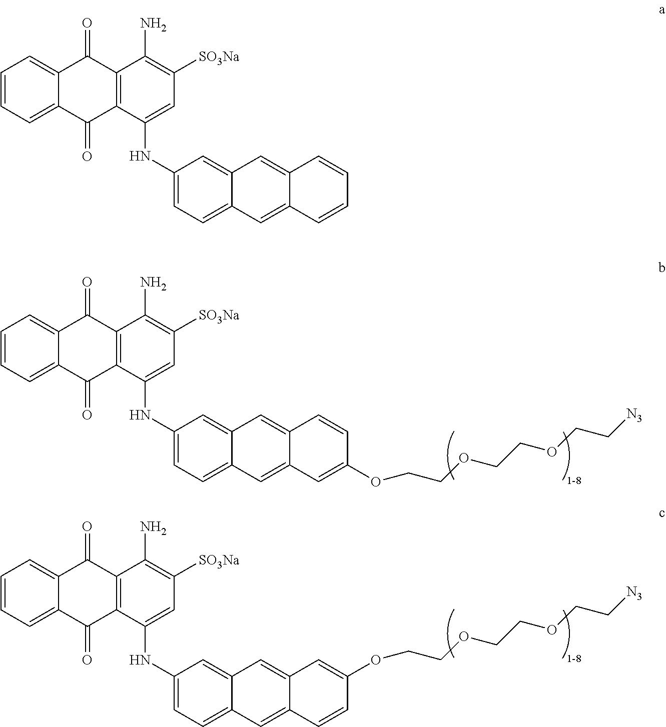

Arginase Inhibitors

In some embodiments, dendrimers are associated with or conjugated to one or more arginase inhibitors. Expression of the enzyme arginase 1 (Arg1) is a defining feature of immunosuppressive myeloid cells and leads to depletion of L-arginine, a nutrient required for T cell and natural killer (NK) cell proliferation. Blocking Arg1 activity in the context of cancer could therefore shift the balance of L-arginine metabolism to favor lymphocyte proliferation. Indeed, in murine studies, injection of the arginase inhibitor nor-NOHA or genetic disruption of Arg1 in the myeloid compartment resulted in reduced tumor growth, indicating that Arg1 is pro-tumorigenic.

Thus, in some embodiments, dendrimers are associated with or conjugated to one or more arginase inhibitors such as boronic acid-based arginase inhibitors, for example, derivatives of 2-(S)-amino-6-boronohexanoic acid (ABH) (Borek B et al., Bioorg Med Chem. 2020 Sep. 15; 28(18):115658), or derivatives, analogs or prodrugs, or pharmacologically active salts thereof. In preferred embodiments, dendrimers are conjugated to one or more arginase inhibitors or derivatives, analogues or prodrugs, or pharmacologically active salts thereof. Arginase inhibitors can be functionalized, for example with ether, ester, amine, or amide linkage, optionally with one or more spacers/linkers, for ease of conjugation with the dendrimers and/or for desired release kinetics. In preferred embodiments, arginase inhibitors or derivatives, analogs or prodrugs thereof, are conjugated to the dendrimers via Cu (I) catalyzed alkyne-azide click or thiol-ene click chemistry.

In some embodiments, one or more arginase inhibitors and/or derivatives or analogs thereof having structures as shown in Structure XXIV a-g and Structure XXV a-h below are conjugated to dendrimers.

### Structure XXIV a-g: Structures of Arginase Inhibitors and Analogs Thereof

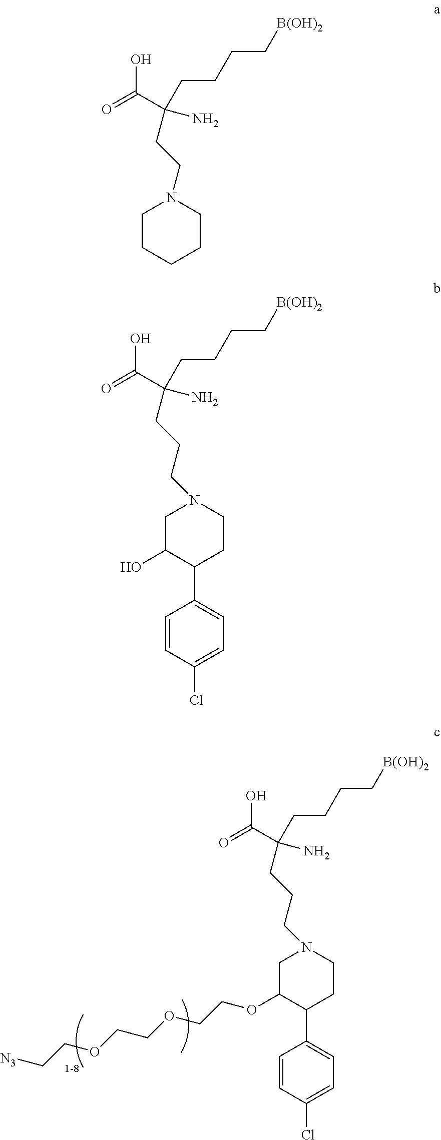

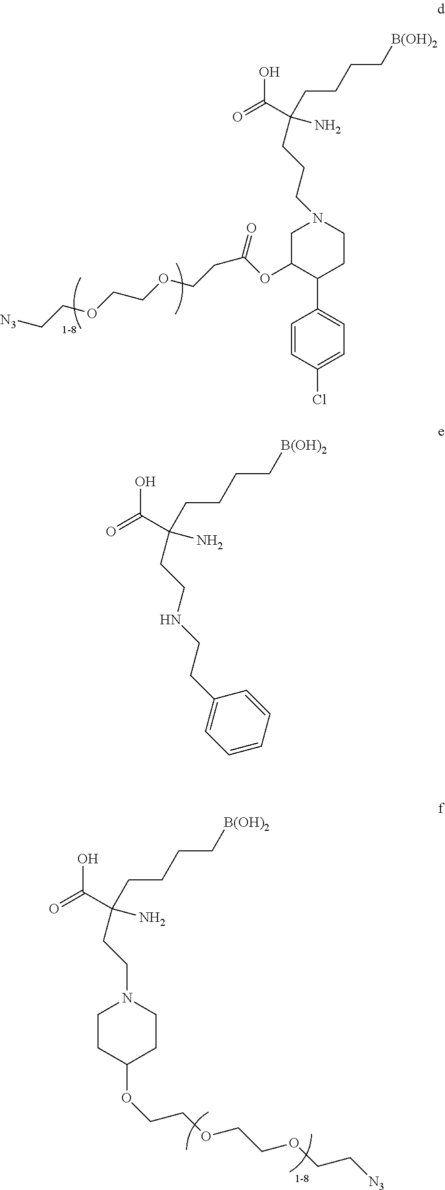

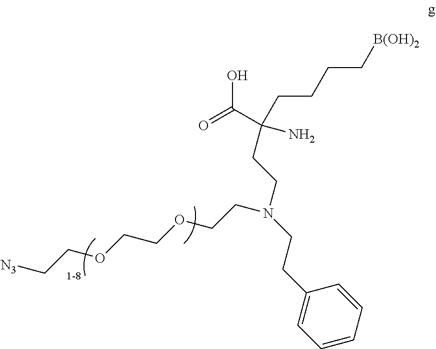

### Structure XXV a-h: Structures of Arginase Inhibitors and Analogs Thereof

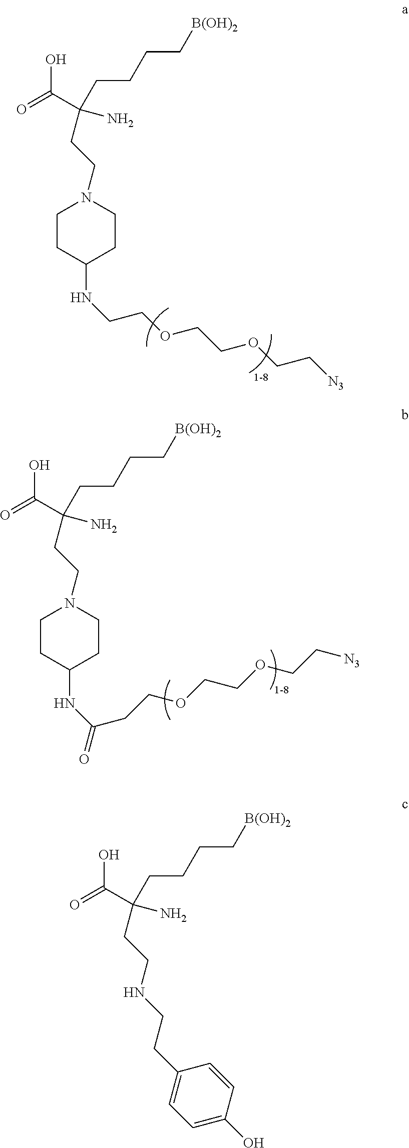

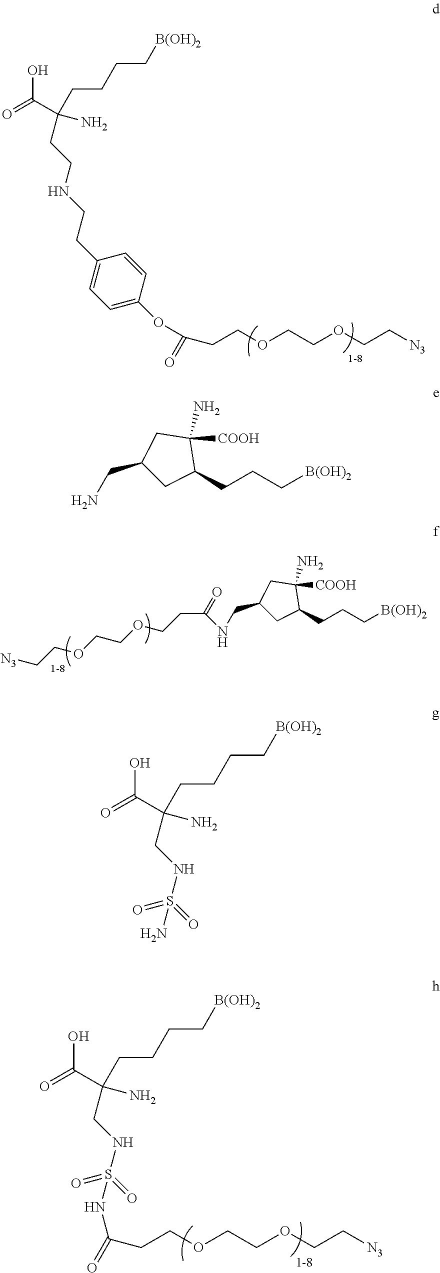

Phosphatidylinositol-3-kinase (PI3K) Inhibitors

In some embodiments, dendrimers are associated with or conjugated to one or more PI3K inhibitors. Dysregulation of PI3K/PTEN pathway components, resulting in hyperactivated PI3K signaling, is frequently observed in various cancers and correlates with tumor growth and survival. Resistance to a variety of anticancer therapies, including receptor tyrosine kinase (RTK) inhibitors and chemotherapeutic agents, has been attributed to the absence or attenuation of downregulating signals along the PI3K/PTEN pathway. Macrophage PI 3-kinase γ controls a critical switch between immune stimulation and suppression during inflammation and cancer. PI3Kγ signaling through Akt and mTor inhibits NFκB activation while stimulating C/EBPβ activation, thereby inducing a transcriptional program that promotes immune suppression during inflammation and tumor growth. By contrast, selective inactivation of macrophage PI3Kγ stimulates and prolongs NFκB activation and inhibits C/EBPβ activation, thus promoting an immunostimulatory transcriptional program that restores CD8+ T cell activation and cytotoxicity.

Thus, in some embodiments, dendrimers are associated with or conjugated to one or more PI3K inhibitor, such as one or more PI3K γ inhibitors. Exemplary PI3K inhibitors include BYL719 (alpelisib), INK1117 (serabelisib, MLN-1117 or TAK-117), XL147 (SAR245408), pilaralisib, WX-037, NVP-BEZ235 (dactolisib or BEZ235), LY3023414 (prexasertib), XL765 (voxtalisib or SAR245409), PX-866, ZSTK474, NVP-BKM120 (buparlisib), GDC-0941(pictilisib), and BAY80-6946 (copanlisib). The PI3K inhibitors can be functionalized, for example with ether, ester, or amide linkage, optionally with one or more spacers/linkers, for ease of conjugation with the dendrimers and/or for desired release kinetics. In preferred embodiments, the PI3K inhibitors or derivatives, analogs or prodrugs thereof, are conjugated to the dendrimers via Cu (I) catalyzed alkyne-azide click or thiol-ene click chemistry, optionally via one or more spacers/linkers such as polyethylene glycol (PEG). The chemical structure of exemplary PI3K inhibitors is shown below as Structure XXVI and Structure XXVII.

### Structure XXVI a-k: Structures of PI3K Inhibitors and Analogs Thereof

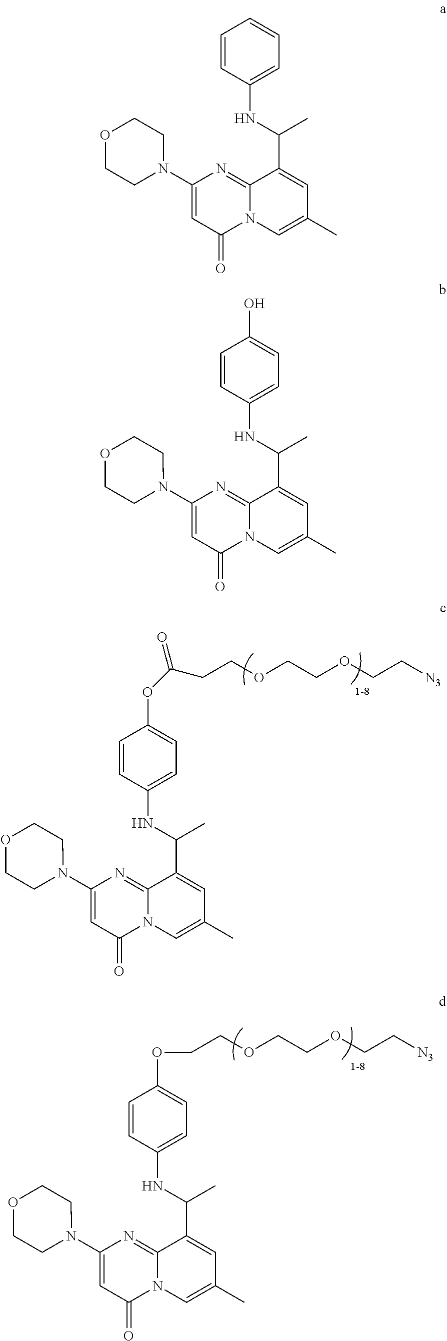

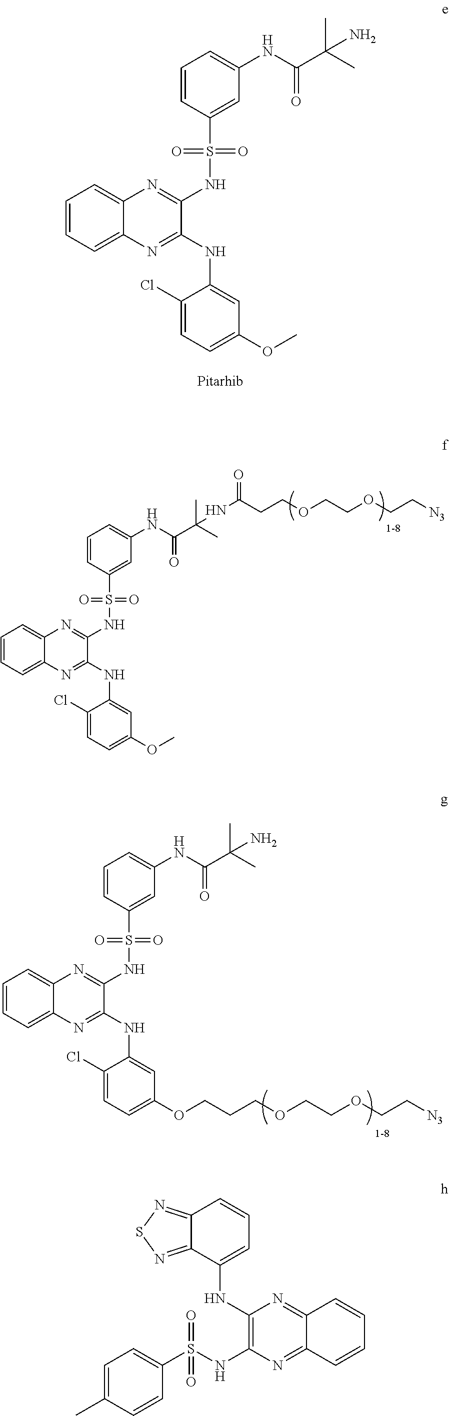

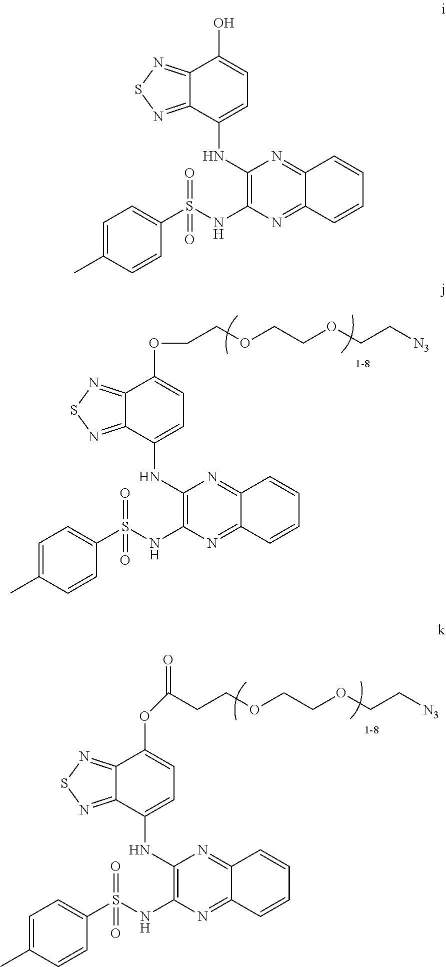

### Structure XXVII a-f: Structures of PI3K Inhibitors and Analogs Thereof

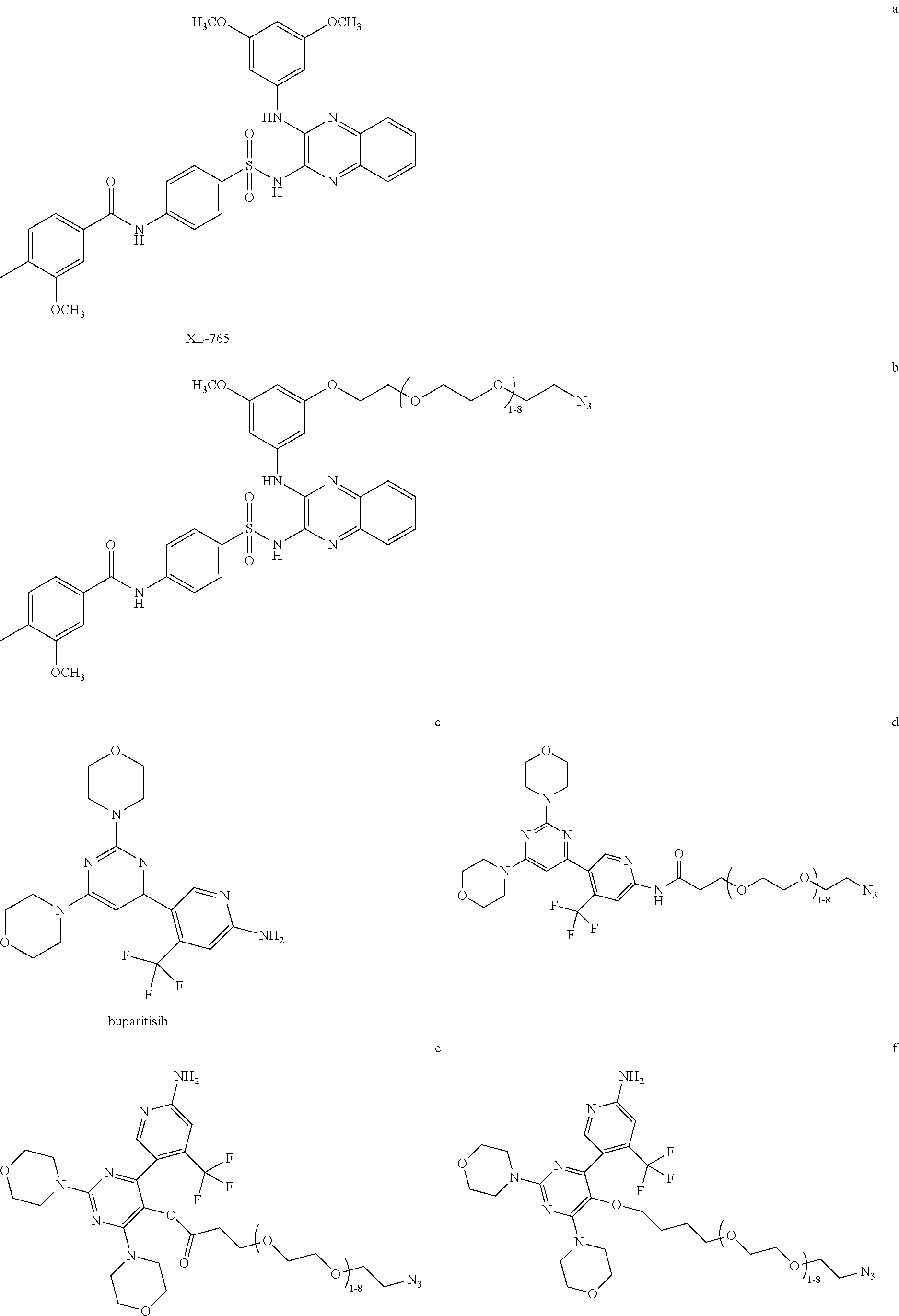

### Toll-like Receptor 4 (TLR4) and TLR7 Agonists

In some embodiments, dendrimers are associated with or conjugated to one or more Toll-like Receptor 4 (TLR4) and/or TLR7 Agonists. TLRs play a vital role in activating immune responses. TLRs recognize conserved pathogen-associated molecular patterns (PAMPs) expressed on a wide array of microbes, as well as endogenous DAMPs released from stressed or dying cells.

In some embodiments, dendrimers are associated with or conjugated to one or more TLR4 agonists. Exemplary TLR4 agonists include synthetic toll-like receptor 4 agonist glucopyranosyl lipid A, Bacillus Calmette-Guérin (BCG) and monophosphoryl lipid A (MPLA). The TLR4 agonists can be functionalized, for example with ether, ester, or amide linkage, optionally with one or more spacers/linkers, for ease of conjugation with the dendrimers and/or for desired release kinetics. In some embodiments, the dendrimers are generation 4, 5, or 6 hydroxyl-terminated PAMAM dendrimers. In preferred embodiments, the TLR4 agonists or derivatives, analogues or prodrugs thereof, are conjugated to dendrimers via Cu (I) catalyzed alkyne-azide click or thiol-ene click chemistry, optionally via one or more spacers/linkers such as polyethylene glycol (PEG). Exemplary TLR4 agonists or analogues thereof are shown below.

### Structure XXVIII a-b: Structures of Two TLR4 Agonist Analogues

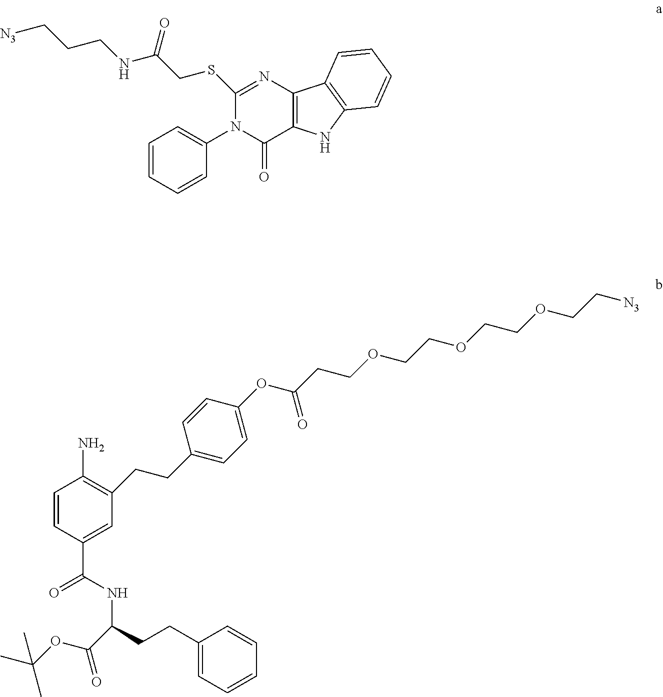 [SMILES: [N-]=[N+]=NCCCNC(=O)CSc1nc2c([nH]c3ccccc32)c(=O)n1-c1ccccc1 ; CC(C)(C)OC(=O)[C@H](CCc1ccccc1)NC(=O)c1ccc(N)c(CCc2ccc(OC(=O)CCOCCOCCOCCN=[N+]=[N-])cc2)c1]

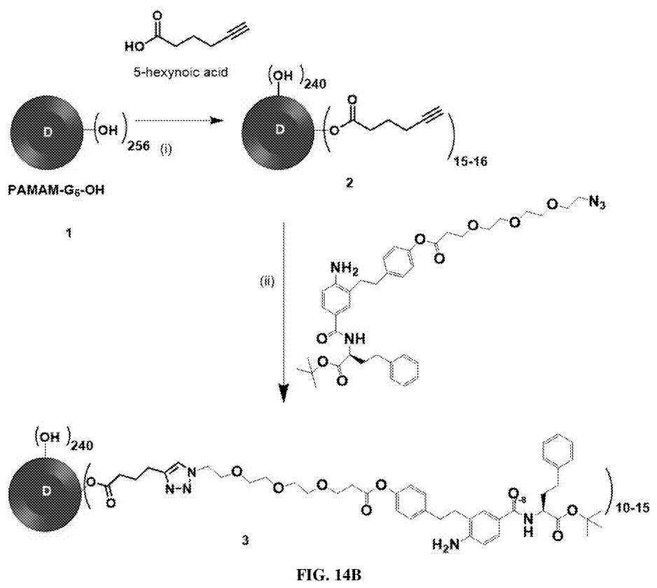

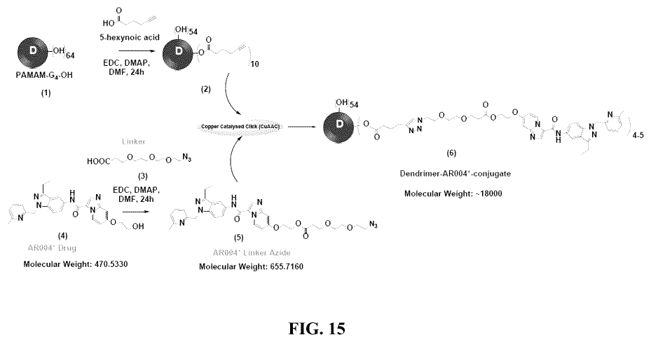

The chemical synthesis routes of exemplary TLR4 agonists conjugated to dendrimers are shown in FIGS. 14A and 14B.

In some embodiments, dendrimers are associated with or conjugated to one or more TLR7 agonists. Exemplary TLR7 agonists include imiquimod, resiquimod, gardiquimod, 852A, Loxoribine, Bropirimine, 3M-011, 3M-052, DSR-6434, DSR-29133, SC1, SZU-101, SM-276001, and SM-360320. In preferred embodiments, the TLR agonist is resiquimod. The TLR7 agonists can be functionalized, for example with ether, ester, or amide linkage, optionally with one or more spacers/linkers, for ease of conjugation with the dendrimers and/or for desired release kinetics.

In some embodiments, dendrimers associated with or conjugated to one or more TLR4 or TLR7 agonists are used in combination with anti-tumor vaccines and/or adoptive cell therapy (ACT) as an adjuvant, for example to increase expression of innate immune genes, infiltration and expansion of activated effector T cells, antigen presentation, and durable immune responses.

SHP2 Inhibitors

SHP2 (Src homology-2 domain-containing protein tyrosine phosphatase-2) is a non-receptor protein tyrosine phosphatase that removes tyrosine phosphorylation. Functionally, SHP2 serves as an important hub to connect several intracellular oncogenic signaling pathways, such as Jak/STAT, PI3K/AKT, RAS/Raf/MAPK, and PD-1/PD-L1 pathways. Mutations and/or overexpression of SHP2 has been associated with genetic developmental diseases and cancers.

Hence, in some embodiments, dendrimers are associated with or conjugated to one or more SHP2 inhibitors, or derivatives, analogs or prodrugs, or pharmacologically active salts thereof. Exemplary SHP2 inhibitors include inhibitors targeting the catalytic site and inhibitors targeting the allosteric site of SHP2, for example, TN0155, RMC-4630, JAB-3068, JAB-3312, and RMC-4550. SHP2 inhibitors can be functionalized, for example with ether, ester, or amide linkage, optionally with one or more spacers/linkers, for ease of conjugation with the dendrimers and/or for desired release kinetics. In some embodiments, the dendrimers are generation 4, 5, or 6 hydroxyl-terminated PAMAM dendrimers. In preferred embodiments, the SHP2 inhibitors or derivatives, analogs or prodrugs thereof, are conjugated to dendrimers via Cu (I) catalyzed alkyne-azide click or thiol-ene click chemistry, optionally via one or more spacers/linkers such as polyethylene glycol (PEG). Exemplary SHP2 inhibitors or analogues thereof are shown below.

### Structure XXIX a-b: Structures of Two SHP2 Inhibitor Analogues

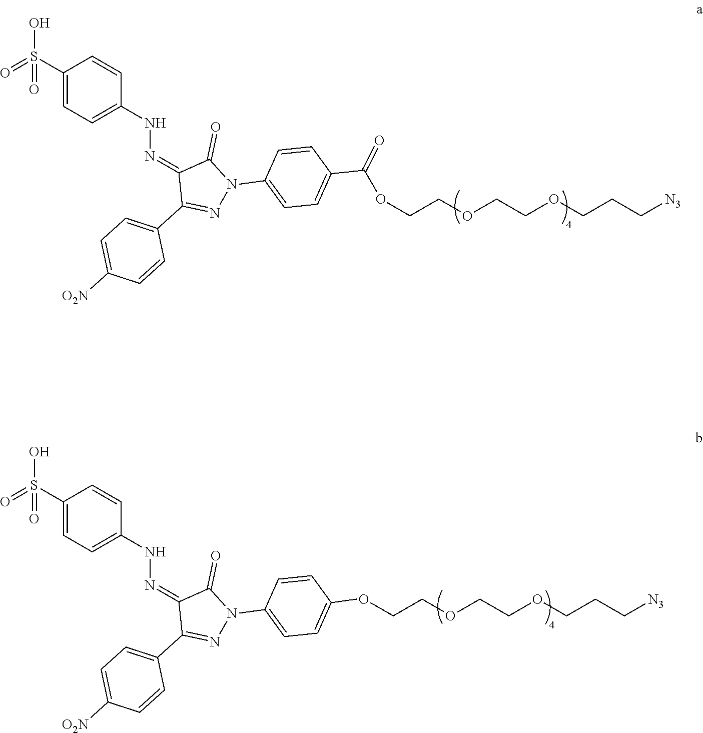

Some exemplary immunomodulatory agents used with dendrimers also include STING antagonists, JAK1 inhibitors, and anti-inflammatory agents. In preferred embodiments, dendrimers associated with or conjugated to one or more immunomodulatory agents including STING antagonists, JAK1 inhibitors, and anti-inflammatory agents are particularly suited for targeting one or more pro-inflammatory immune cells.

STING Antagonists

In some embodiments, dendrimers are conjugated to one or more STING antagonists. STING activation elicits a Type-1 Interferon response. In the case of autoimmune diseases, STING antagonists (turning STING “off”) may have therapeutic potential in Type-I interferonopathies, such as SLE (lupus), where STING drives an exaggerated interferon response.

Thus, in some embodiments, dendrimers are conjugated to one or more STING antagonists including C-178, C-176, C18, Astin C, No₂-cLA, and H-151. In one embodiment, dendrimers are conjugated to H-151, or a derivative, analog or prodrug, or a pharmacologically active salt thereof. The STING antagonists can be functionalized, for example, with ether, ester, or amide linkage, optionally with one or more spacers/linkers, for ease of conjugation with the dendrimers and/or for desired release kinetics. In preferred embodiments, the STING antagonists or derivatives, analogs or prodrugs thereof, are conjugated to the dendrimers via Cu (I) catalyzed alkyne-azide click or thiol-ene click chemistry, optionally via one or more spacers/linkers such as polyethylene glycol (PEG). Exemplary STING antagonists are shown below.

### Structure XXX a-f: Human and Mouse STING Antagonists

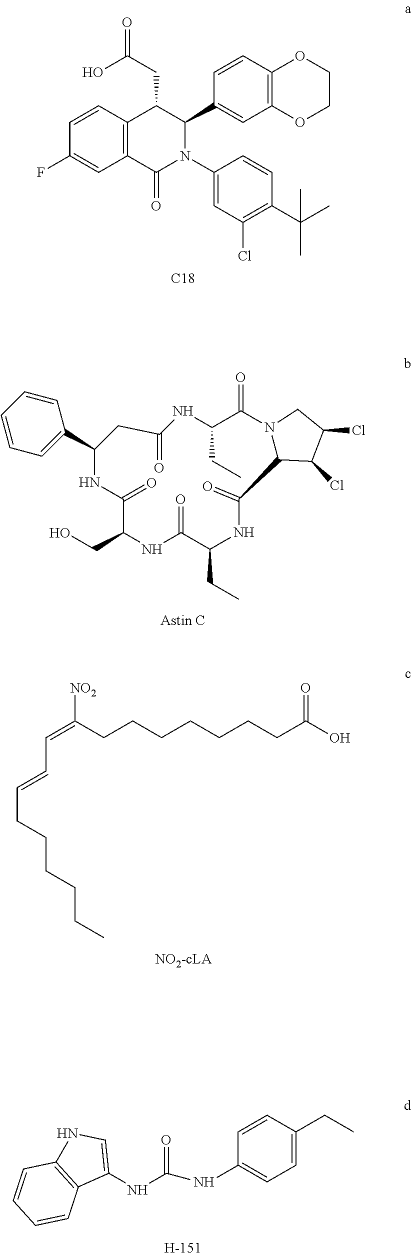

### Mouse STING Antagonists

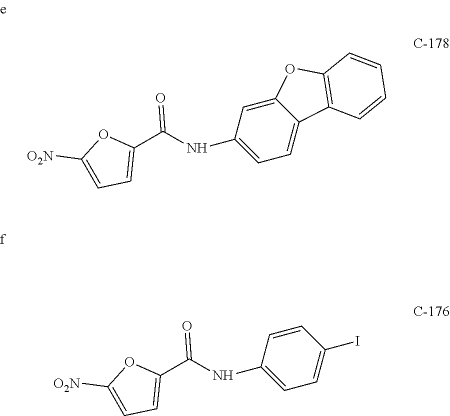 [SMILES: O=C(Nc1ccc2c(c1)oc1ccccc12)c1ccc([N+](=O)[O-])o1 ; O=C(Nc1ccc(I)cc1)c1ccc([N+](=O)[O-])o1]

In some embodiments, the STING antagonist is alpha-mangostin (structure shown below).

### Structure XXXI: Alpha Mangostin

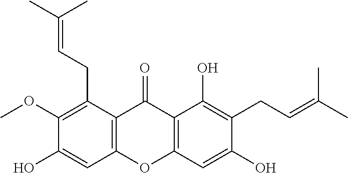 [SMILES: COc1c(O)cc2oc3cc(O)c(CC=C(C)C)c(O)c3c(=O)c2c1CC=C(C)C]

JAK1 Inhibitors

Janus kinase (JAK)/signal transducers and activators of transcription (STATs) are a group of molecules associated with one of the major pathways through which many cytokines exert and integrate their function, and as such they are increasingly recognized as playing critical role in the pathogenesis subserving various immune-mediated diseases, including RA, PsA, SpAs, IBD, skin disorders (e.g. alopecia areata, atopic dermatitis), single-gene disorders like interferonopathies, and others. JAKs are the key initiating players of the JAK/STAT pathway. Upon binding of their respective effector molecules (cytokines, IFNs, growth factors and others) to type I and type II receptors, JAKs are activated, and through phosphorylation of themselves and of other molecules (including STATs), they mediate signal transduction to the nucleus. A class of drugs, called JAK inhibitors or JAKinibs that block one or more JAKs has been developed in the last decade.

Exemplary JAK inhibitors include tofacitinib, ruxolitinib, baricitinib, peficitinib, decernotiniba, filgotinib, solcitinibb, itacitinib, SHR0302, upadacitinib, PF-04965842. Tofacitinib, a first-generation JAKinib that inhibits JAK3, JAK1, and to a lesser degree JAK2, is the first JAKinib developed for the treatment of autoimmune disease. Baricitinib is a first-generation JAKinib with activity against JAK1 and JAK2 that is structurally related to ruxolitinib. Peficitinib blocks all four JAK isoforms but has slight JAK3 selectivity.

In some embodiments, the dendrimers are associated with or conjugated to one or more JAK inhibitors. In some embodiments, the dendrimers conjugated to one or more JAK1 inhibitors are formulated for treating or alleviating one or more symptoms of one or more chronic inflammatory conditions such as rheumatoid arthritis, psoriatic disease, spondyloarthropathies, and Inflammatory bowel disease (IBD).

JAK1 inhibitors can be functionalized, for example, with ether, ester, or amide linkage, optionally with one or more spacers/linkers, for ease of conjugation with the dendrimers and/or for desired release kinetics. In preferred embodiments, the JAK1 inhibitors or derivatives, analogs or prodrugs thereof, are conjugated to the dendrimers via Cu (I) catalyzed alkyne-azide click or thiol-ene click chemistry, optionally via one or more spacers/linkers such as polyethylene glycol (PEG).

In one embodiment, the JAK1 inhibitor complexed or conjugated to a dendrimer is Target-006 (Structure XXXII) or a derivative, analog or prodrug, or a pharmacologically active salt thereof.

### Structure XXXII: Target-007

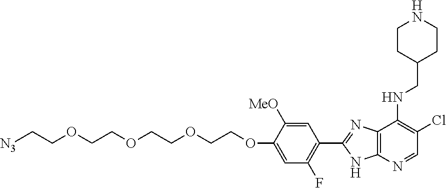 [SMILES: COc1cc(-c2nc3c(NCC4CCNCC4)c(Cl)cnc3[nH]2)c(F)cc1OCCOCCOCCOCCN=[N+]=[N-]]

An exemplary conjugation of JAK1 inhibitor Target-007 to a dendrimer is shown below (Structure XXXIII). JAK1 binding affinity of Target-007 is about 1 nm and the binding affinity of dendrimer conjugated Target-007 is about nm. Thus, in preferred embodiments, the JAK1 inhibitors are conjugated to dendrimers with or without a spacer in such a way that it minimizes the reduction in binding affinity towards JAK1, for example, less than 1-fold, 2-fold, 3-fold, 4-fold, 5-fold, 10-fold, 20-fold, 30-fold, 40-fold, 50-fold, 100-fold, 200-fold, and 500-fold.

### Structure XXXIII: Dendrimer Conjugated Target-007 (D-007)

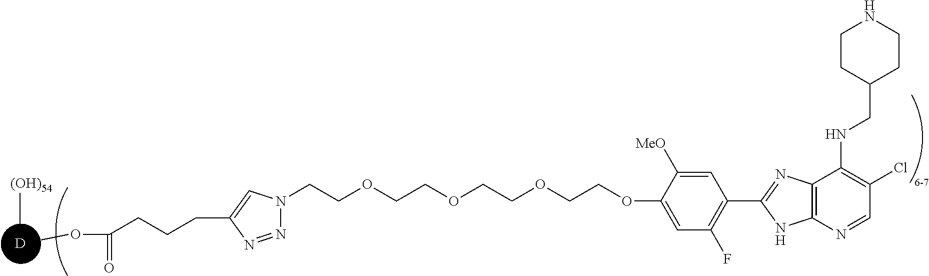

In another embodiment, the JAK1 inhibitor complexed or conjugated to a dendrimer is Target-006 (Structure XXXIV) or a derivative, analog or prodrug, or a pharmacologically active salt thereof.

### Structure XXXIV: Target-006

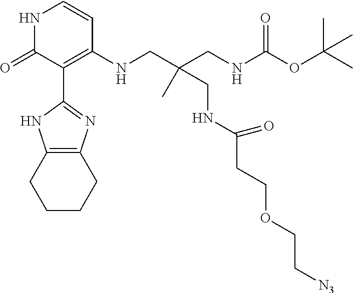 [SMILES: CC(CNC(=O)CCOCCN=[N+]=[N-])(CNC(=O)OC(C)(C)C)CNc1cc[nH]c(=O)c1-c1nc2c([nH]1)CCCC2]

An exemplary conjugation of JAK1 inhibitor Target-006 to a dendrimer is shown below (Structure XXXV).

### Structure XXXV: Dendrimer Conjugated Target-006 (D-006)

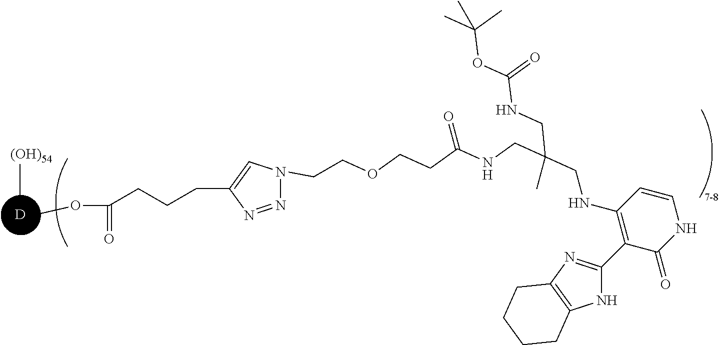

Anti-Inflammatory Agents

In some embodiments, one or more anti-inflammatory agents are associated with or complexed to dendrimers. Anti-inflammatory agents reduce inflammation and include steroidal and non-steroidal drugs. Suitable steroidal active agents include glucocorticoids, progestins, mineralocorticoids, and corticosteroids. In some embodiments, one or more active agents are one or more corticosteroids.

Exemplary anti-inflammatory agents include triamcinolone acetonide, fluocinolone acetonide, methylprednisolone, prednisolone, prednisone, dexamethasone, loteprendol, fluorometholone, ibuprofen, aspirin, and naproxen. Exemplary immune-modulating drugs include cyclosporine, tacrolimus, rapamycin, and metformin. Exemplary non-steroidal anti-inflammatory drugs (NSAIDs) include mefenamic acid, aspirin, Diflunisal, Salsalate, Ibuprofen, Naproxen, Fenoprofen, Ketoprofen, Deacketoprofen, Flurbiprofen, Oxaprozin, Loxoprofen, Indomethacin, Sulindac, Etodolac, Ketorolac, Diclofenac, Nabumetone, Piroxicam, Meloxicam, Tenoxicam, Droxicam, Lornoxicam, Isoxicam, Meclofenamic acid, Flufenamic acid, Tolfenamic acid, elecoxib, Rofecoxib, Valdecoxib, Parecoxib, Lumiracoxib, Etoricoxib, Firocoxib, Sulphonanilides, Nimesulide, Niflumic acid, and Licofelone. In preferred embodiments, the active agent is triamcinolone acetonide, prednisone, dexamethasone, or derivatives, analogues or prodrugs, or pharmacologically active salts thereof. Exemplary analogues of triamcinolone acetonide, prednisone, and dexamethasone are shown below (Structure XXXVI).

### Structure XXXVI a-f: Chemical Structure of Analogues of Triamcinolone Acetonide, Prednisone, Dexamethasone

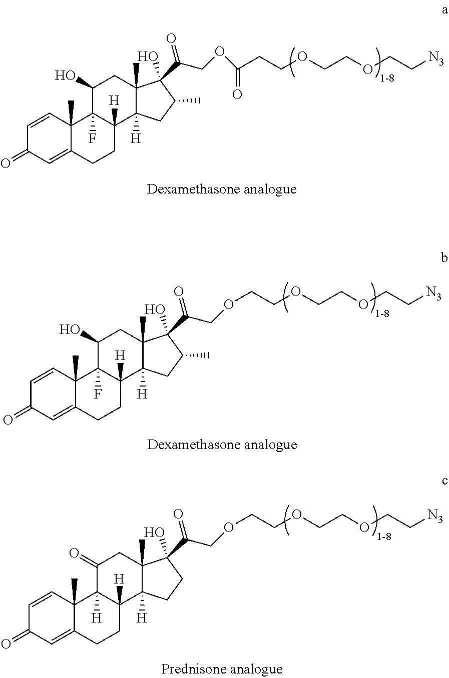

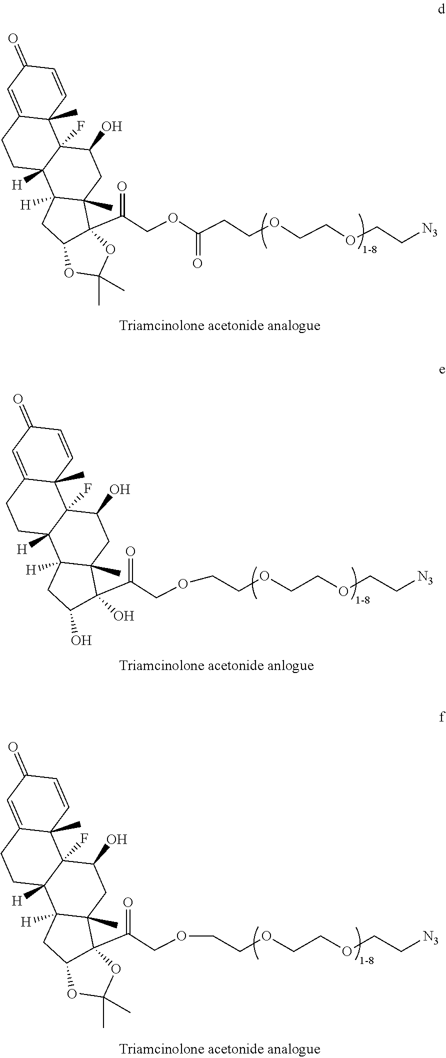

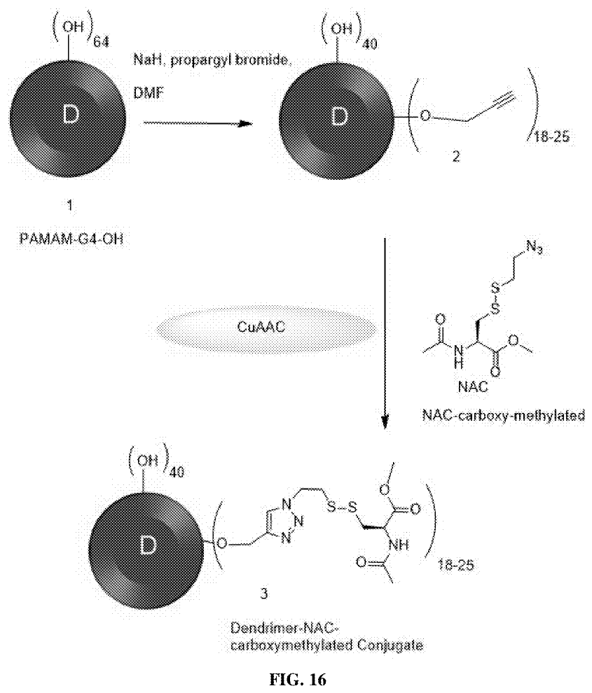

In one embodiment, the active agent is N-acetyl-L-cysteine. In a preferred embodiment, N-acetyl-L-cysteine is conjugated to a hydroxyl-terminated PAMAM dendrimer via non-cleavable linkage for minimal release of free N-acetyl-cysteine in vivo after administration. The synthesis route for an exemplary non-releasable (or non-cleavable) form of the dendrimer/N-acetyl-cysteine complexes is shown in FIG. 16. The non-releasable form of the dendrimer/N-acetyl-cysteine complex provides enhanced therapeutic efficacy as compared to a releasable or cleavable form of the dendrimer/N-acetyl-cysteine complex.

In some embodiments, one or more active agents are polysialic acid (e.g., low molecular weight polySia with an average degree of polymerization (polySia avDP20)), Translocator Protein Ligands (e.g., Diazepam binding inhibitor (DBI)), Interferon-β (IFN-β), and minocycline.

In some cases, one or more active agents are anti-infective agents. Exemplary anti-infectious agents include antiviral agents, antibacterial agents, antiparasitic agents, and anti-fungal agents. Exemplary antibiotics include moxifloxacin, ciprofloxacin, erythromycin, levofloxacin, cefazolin, vancomycin, tigecycline, gentamycin, tobramycin, ceftazidime, ofloxacin, gatifloxacin; antifungals: amphotericin, voriconazole, natamycin.

D. Additional Active Agents to be Delivered

In some embodiments, the dendrimers are used to deliver one or more additional active agents, particularly one or more active agents to prevent or treat one or more symptoms of proliferative diseases. Suitable therapeutic, diagnostic, and/or prophylactic agents can be a biomolecule, such as an enzyme, protein, polypeptide, or nucleic acid or a small molecule agent (e.g., molecular weight less than 2000 amu, preferably less than 1500 amu), including organic, inorganic, and organometallic agents. The agent can be encapsulated within the dendrimers, dispersed within the dendrimers, and/or associated with the surface of the dendrimer, either covalently or non-covalently.

1. Therapeutic Agents

In some embodiments, the dendrimer complexes include one or more therapeutic, prophylactic, or prognostic agents that are complexed or conjugated to the dendrimers. Representative therapeutic agents include, but are not limited to, chemotherapeutic agents, anti-infectious agents, and combinations thereof.

Additional therapeutic agents include conventional cancer therapeutics such as chemotherapeutic agents, cytokines, chemokines, and radiation therapy. The majority of chemotherapeutic drugs can be divided into alkylating agents, antimetabolites, anthracyclines, plant alkaloids, topoisomerase inhibitors, and other antitumour agents. These drugs affect cell division or DNA synthesis and function in some way. Additional therapeutics include monoclonal antibodies and the tyrosine kinase inhibitors e.g., imatinib mesylate (GLEEVEC® or GLIVEC®), which directly targets a molecular abnormality in certain types of cancer (chronic myelogenous leukemia, gastrointestinal stromal tumors).

Representative chemotherapeutic agents include, but are not limited to, amsacrine, bleomycin, busulfan, camptothecin, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epipodophyllotoxins, epirubicin, etoposide, etoposide phosphate, fludarabine, fluorouracil, gemcitabine, hydroxycarb amide, idarubicin, ifosfamide, innotecan, leucovorin, liposomal doxorubicin, liposomal daunorubici, lomustine, mechlorethamine, melphalan, mercaptopurine, mesna, methotrexate, mitomycin, mitoxantrone, oxaliplatin, paclitaxel, pemetrexed, pentostatin, procarbazine, raltitrexed, satraplatin, streptozocin, teniposide, tegafur-uracil, temozolomide, teniposide, thiotepa, tioguanine, topotecan, treosulfan, vinblastine, vincristine, vindesine, vinorelbine, vorinostat, taxol, trichostatin A and derivatives thereof, trastuzumab (HERCEPTIN®), cetuximab, and rituximab (RITUXAN® or MABTHERA®), bevacizumab (AVASTIN®), and combinations thereof. Representative pro-apoptotic agents include, but are not limited to, fludarabinetaurosporine, cycloheximide, actinomycin D, lactosylceramide, 15d-PGJ(2)5 and combinations thereof.

In some embodiments, the active agents are histone deacetylase (HDAC) inhibitors. In one embodiment, the active agent is vorinostat. In other embodiments, the active agents are topoisomerase I and/or II inhibitors. In a particular embodiment, the active agent is etoposide or camptothecin.

Additional anti-cancer agents include, but are not limited to, irinotecan, exemestane, octreotide, carmofur, clarithromycin, zinostatin, tamoxifen, tegafur, toremifene, doxifluridine, nimustine, vindensine, nedaplatin, pirarubicin, flutamide, fadrozole, prednisone, medroxyprogesterone, mitotane, mycophenolate mofetil, and mizoribine.

Representative anti-angiogenesis agents include, but are not limited to, antibodies to vascular endothelial growth factor (VEGF) such as bevacizumab (AVASTIN®) and rhuFAb V2 (ranibizumab, LUCENTIS®), and other anti-VEGF compounds including aflibercept (EYLEA®); MACUGEN® (pegaptanim sodium, anti-VEGF aptamer or EYE001) (Eyetech Pharmaceuticals); pigment epithelium derived factor(s) (PEDF); COX-2 inhibitors such as celecoxib (CELEBREX®) and rofecoxib (VIOXX®); interferon alpha; interleukin-12 (IL-12); thalidomide (THALOMID®) and derivatives thereof such as lenalidomide (REVLIMID®); squalamine; endostatin; angiostatin; ribozyme inhibitors such as ANGIOZYME® (Sirna Therapeutics); multifunctional antiangiogenic agents such as NEOVASTAT® (AE-941) (Aeterna Laboratories, Quebec City, Canada); receptor tyrosine kinase (RTK) inhibitors such as sunitinib (SUTENT®); tyrosine kinase inhibitors such as sorafenib (Nexavar®) and erlotinib (Tarceva®); antibodies to the epidermal grown factor receptor such as panitumumab (VECTIBIX®) and cetuximab (ERBITUX®), as well as other anti-angiogenesis agents known in the art.

In some cases, the active agent is an anti-infectious agent. Exemplary anti-infectious agents include antiviral agents, antibacterial agents, antiparasitic agents, and anti-fungal agents. Exemplary antibiotics include moxifloxacin, ciprofloxacin, erythromycin, levofloxacin, cefazolin, vancomycin, tigecycline, gentamycin, tobramycin, ceftazidime, ofloxacin, gatifloxacin; antifungals: amphotericin, voriconazole, natamycin.

Any of the additional active compounds can be functionalized, for example with ether, ester, ethyl, or amide linkage, optionally with one or more spacers/linkers, for ease of conjugation with the dendrimers and/or for desired release kinetics. In preferred embodiments, active agents or derivatives, analogs or prodrugs thereof, are conjugated to the dendrimers via Cu (I) catalyzed alkyne-azide click or thiol-ene click chemistry, optionally via one or more spacers/linkers such as polyethylene glycol (PEG). In some embodiments, the additional active agents are chemotherapeutic agents or derivatives, analogs or prodrugs, or pharmacologically active salts thereof. In one embodiment, the active agent complexed or conjugated to dendrimer is methotrexate, or a derivative, analog or prodrug, or a pharmacologically active salt thereof, for example as shown in Structure XXXVII.

### Structure XXXVII: Chemical Structure of Methotrexate Analogue

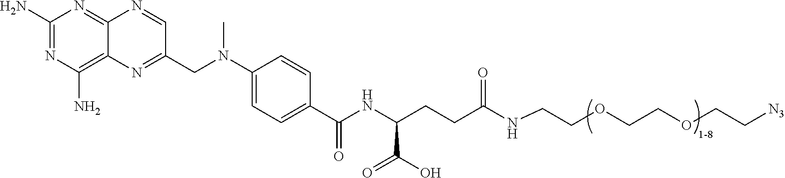

2. Diagnostic Agents

In some embodiments, the dendrimers are conjugated to or complexed with one or more diagnostic agents. Examples of diagnostic agents include paramagnetic molecules, fluorescent compounds, magnetic molecules, and radionuclides, x-ray imaging agents, and contrast media. Examples of other suitable contrast agents include gases or gas emitting compounds, which are radioopaque. Dendrimer complexes can further include agents useful for determining the location of administered compositions. Agents useful for this purpose include fluorescent tags, radionuclides and contrast agents.

Exemplary diagnostic agents include dyes, fluorescent dyes, Near infra-red dyes, SPECT imaging agents, PET imaging agents and radioisotopes. Representative dyes include carbocyanine, indocarbocyanine, oxacarbocyanine, thüicarbocyanine and merocyanine, polymethine, coumarine, rhodamine, xanthene, fluorescein, boron-dipyrromethane (BODIPY), Cy5, Cy5.5, Cy7, VivoTag-680, VivoTag-5680, VivoTag-5750, AlexaFluor660, AlexaFluor680, AlexaFluor700, AlexaFluor750, AlexaFluor790, Dy677, Dy676, Dy682, Dy752, Dy780, DyLight547, Dylight647, HiLyte Fluor 647, HiLyte Fluor 680, HiLyte Fluor 750, IRDye 800CW, IRDye 800R5, IRDye 700DX, ADS780WS, ADS830WS, and ADS832WS.

Exemplary SPECT or PET imaging agents include chelators such as diethylene tri-amine penta-acetic acid (DTPA), 1,4,7,10-tetra-azacyclododecane-1,4,7,10-tetraacetic acid (DOTA), di-amine dithiols, activated mercaptoacetyl-glycyl-glycyl-gylcine (MAG3), and hydrazidonicotinamide (HYNIC).

Exemplary isotopes include Tc-94m, Tc-99m, In-111, Ga-67, Ga-68, Gd3+, Y-86, Y-90, Lu-177, Re-186, Re-188, Cu-64, Cu-67, Co-55, Co-57, F-18, Sc-47, Ac-225, Bi-213, Bi-212, Pb-212, Sm-153, Ho-166, and Dy-166.

In preferred embodiments, the dendrimer complex include one or more radioisotopes suitable for positron emission tomography (PET) imaging. Exemplary positron-emitting radioisotopes include carbon-11 (¹¹C), copper-64 (⁶⁴Cu), nitrogen-13 (¹³N), oxygen-15 (¹⁵O), gallium-68 (⁶⁸Ga), and fluorine-18 (¹⁸F) e.g., 2-deoxy-2-¹⁸F-fluoro-β-D-glucose (¹⁸F-FDG).

In further embodiments, a singular dendrimer complex composition can simultaneously treat and/or diagnose a disease or a condition at one or more locations in the body, for example, at primary tumor site and metastasized sites.

3. Targeting or Binding Moieties

In some embodiments, the dendrimer includes one or more tissue targeting or tissue binding moieties, for targeting the dendrimer to a specific location in vivo, and/or for enhancing the in vivo residence time at a desired location within the body. For example, in some embodiments, the dendrimer is sequestered or bound to one or more distinct tissues or organs following local or systemic administration into the body. Therefore, the presence of a targeting or binding moiety can enhance the delivery of an active agent to a target site relative to the dendrimer and active agent in the absence of a targeting or binding moiety. Conjugation of the dendrimer to one or more targeting or binding moieties can be via a spacer, and the linkage between the spacer and dendrimer, and/or the spacer and targeting agent can be designed to provide releasable or non-releasable forms of the dendrimer-targeting agent complex.

An exemplary targeting agent is alendronic acid (alendronate), which binds to hypoxyapetite at the surface of bones, and enhances the residence tine of the dendrimer complex to bones. Alendronate is a small molecule targeting moiety, which selectively binds to hydroxyapatite, a component of bone. Therefore, in some embodiments, the dendrimer is conjugated to alendronate, for selective targeting of the dendrimer to bone. In some embodiments, the conjugation between the alendronate and the dendrimer is via a reversible (non-covalent) linker. In other embodiments, the conjugation between the alendronate and the dendrimer is via a non-cleavable or a minimally cleavable linker. In some embodiments, the targeting agent also has a therapeutic effect at the targeted site. In some embodiments, the dendrimer is conjugated to alendronate, for targeting the dendrimer complex to bone and for providing a therapeutic effect at the site of bone inflammation. In some embodiments, alendronate-bound dendrimers are conjugated to one or more active agents for selective delivery of the active agents to sites of bone inflammation.

### III. Pharmaceutical Formulations

Pharmaceutical compositions including one or more dendrimer complexes may be formulated in a conventional manner using one or more physiologically acceptable carriers including excipients and auxiliaries which facilitate processing of the active compounds into preparations which can be used pharmaceutically. Proper formulation is dependent upon the route of administration chosen. In preferred embodiments, the compositions are formulated for parenteral delivery. In some embodiments, the compositions are formulated for intratumoral injection. Typically the compositions will be formulated in sterile saline or buffered solution for injection into the tissues or cells to be treated. The compositions can be stored lyophilized in single use vials for rehydration immediately before use. Other means for rehydration and administration are known to those skilled in the art.

Pharmaceutical formulations contain one or more dendrimer complexes in combination with one or more pharmaceutically acceptable excipients. Representative excipients include solvents, diluents, pH modifying agents, preservatives, antioxidants, suspending agents, wetting agents, viscosity modifiers, tonicity agents, stabilizing agents, and combinations thereof. Suitable pharmaceutically acceptable excipients are preferably selected from materials which are generally recognized as safe (GRAS), and may be administered to an individual without causing undesirable biological side effects or unwanted interactions.

Generally, pharmaceutically acceptable salts can be prepared by reaction of the free acid or base forms of an active agent with a stoichiometric amount of the appropriate base or acid in water or in an organic solvent, or in a mixture of the two; generally, non-aqueous media like ether, ethyl acetate, ethanol, isopropanol, or acetonitrile are preferred. Pharmaceutically acceptable salts include salts of an active agent derived from inorganic acids, organic acids, alkali metal salts, and alkaline earth metal salts as well as salts formed by reaction of the drug with a suitable organic ligand (e.g., quaternary ammonium salts). Lists of suitable salts are found, for example, in Remington's Pharmaceutical Sciences, 20th ed., Lippincott Williams & Wilkins, Baltimore, M D, 2000, p. 704. Examples of drugs sometimes administered in the form of a pharmaceutically acceptable salt include timolol maleate, brimonidine tartrate, and sodium diclofenac.

The compositions are preferably formulated in dosage unit form for ease of administration and uniformity of dosage. The phrase “dosage unit form” refers to a physically discrete unit of conjugate appropriate for the patient to be treated. It will be understood, however, that the total single administration of the compositions will be decided by the attending physician within the scope of sound medical judgment. The therapeutically effective dose can be estimated initially either in cell culture assays or in animal models, usually mice, rabbits, dogs, or pigs. The animal model is also used to achieve a desirable concentration range and route of administration. Such information should then be useful to determine useful doses and routes for administration in humans. Therapeutic efficacy and toxicity of conjugates can be determined by standard pharmaceutical procedures in cell cultures or experimental animals, e.g., ED50 (the dose is therapeutically effective in 50% of the population) and LD50 (the dose is lethal to 50% of the population). The dose ratio of toxic to therapeutic effects is the therapeutic index and it can be expressed as the ratio, LD50/ED50. Pharmaceutical compositions which exhibit large therapeutic indices are preferred. The data obtained from cell culture assays and animal studies can be used in formulating a range of dosages for human use.

In certain embodiments, the compositions are administered locally, for example, by injection directly into a site to be treated. In some embodiments, the compositions are injected, topically applied, or otherwise administered directly into the vasculature onto vascular tissue at or adjacent to a site of injury, surgery, or implantation. For example, in embodiments, the compositions are topically applied to vascular tissue that is exposed, during a surgical or implantation, or transplantation procedure. Typically, local administration causes an increased localized concentration of the compositions which is greater than that which can be achieved by systemic administration.

Pharmaceutical compositions formulated for administration by parenteral (intramuscular, intraperitoneal, intravenous (IV) or subcutaneous injection), enteral, and topical routes of administration are described.

A. Parenteral Administration

In some embodiments, the dendrimers are formulated to be administered parenterally. The phrases “parenteral administration” and “administered parenterally” are art-recognized terms, and include modes of administration other than enteral and topical administration, such as injections, and include without limitation intravenous, intramuscular, intrapleural, intravascular, intrapericardial, intraarterial, intrathecal, intracapsular, intraorbital, intracardiac, intradennal, intraperitoneal, transtracheal, subcutaneous, subcuticular, intraarticular, subcapsular, subarachnoid, intraspinal and intrastemal injection and infusion. In some embodiments, the dendrimers are administered parenterally, for example, by subdural, intravenous, intrathecal, intraventricular, intraarterial, intra-articular, intra-synovial, intra-amniotic, intraperitoneal, or subcutaneous routes.

For liquid formulations, pharmaceutically acceptable carriers may be, for example, aqueous or non-aqueous solutions, suspensions, emulsions or oils. Parenteral vehicles (for subcutaneous, intravenous, intraarterial, or intramuscular injection) include, for example, sodium chloride solution, Ringer's dextrose, dextrose and sodium chloride, lactated Ringer's and fixed oils. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media. The dendrimers can also be administered in an emulsion, for example, water in oil. Examples of oils are those of petroleum, animal, vegetable, or synthetic origin, for example, peanut oil, soybean oil, mineral oil, olive oil, sunflower oil, fish-liver oil, sesame oil, cottonseed oil, corn oil, olive, petrolatum, and mineral. Suitable fatty acids for use in parenteral formulations include, for example, oleic acid, stearic acid, and isostearic acid. Ethyl oleate and isopropyl myristate are examples of suitable fatty acid esters.

Formulations suitable for parenteral administration can include antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Intravenous vehicles can include fluid and nutrient replenishers, electrolyte replenishers such as those based on Ringer's dextrose. In general, water, saline, aqueous dextrose and related sugar solutions, and glycols such as propylene glycols or polyethylene glycol are preferred liquid carriers, particularly for injectable solutions.

Injectable pharmaceutical carriers for injectable compositions are well-known to those of ordinary skill in the art (see, e.g., Pharmaceutics and Pharmacy Practice, J. B. Lippincott Company, Philadelphia, PA, Banker and Chalmers, eds., pages 238-250 (1982), and ASHP Handbook on Injectable Drugs, Trissel, 15th ed., pages 622-630 (2009)).

B. Enteral Administration

In some embodiments, the dendrimers are formulated to be administered enterally. The carriers or diluents may be solid carriers or diluents for solid formulations, liquid carriers or diluents for liquid formulations, or mixtures thereof.

For liquid formulations, pharmaceutically acceptable carriers may be, for example, aqueous or non-aqueous solutions, suspensions, emulsions or oils. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media.

Examples of oils are those of petroleum, animal, vegetable, or synthetic origin, for example, peanut oil, soybean oil, mineral oil, olive oil, sunflower oil, fish-liver oil, sesame oil, cottonseed oil, corn oil, olive, petrolatum, and mineral. Suitable fatty acids for use in parenteral formulations include, for example, oleic acid, stearic acid, and isostearic acid. Ethyl oleate and isopropyl myristate are examples of suitable fatty acid esters.

Vehicles include, for example, sodium chloride solution, Ringer's dextrose, dextrose and sodium chloride, lactated Ringer's and fixed oils. Formulations include, for example, aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Vehicles can include, for example, fluid and nutrient replenishers, electrolyte replenishers such as those based on Ringer's dextrose. In general, water, saline, aqueous dextrose and related sugar solutions are preferred liquid carriers. These can also be formulated with proteins, fats, saccharides and other components of infant formulas.

In preferred embodiments, the compositions are formulated for oral administration. Oral formulations may be in the form of chewing gum, gel strips, tablets, capsules or lozenges. Encapsulating substances for the preparation of enteric-coated oral formulations include cellulose acetate phthalate, polyvinyl acetate phthalate, hydroxypropyl methylcellulose phthalate and methacrylic acid ester copolymers. Solid oral formulations such as capsules or tablets are preferred. Elixirs and syrups also are well known oral formulations.

C. Topical Administration

In some embodiments, the dendrimers are formulated to be administered topically. Topical administration can include application directly to exposed tissue, vasculature, mucosa or to tissues or prostheses, for example, during surgery. The preferred tissue for topical administration is tumor.

### IV. Methods of Making

A. Methods of Making Dendrimers

Dendrimers can be prepared via a variety of chemical reaction steps. Dendrimers are usually synthesized according to methods allowing controlling their structure at every stage of construction. The dendritic structures are mostly synthesized by two main different approaches: divergent or convergent.

In some embodiments, dendrimers are prepared using divergent methods, in which the dendrimer is assembled from a multifunctional core, which is extended outward by a series of reactions, commonly a Michael reaction. The strategy involves the coupling of monomeric molecules that possesses reactive and protective groups with the multifunctional core moiety which leads to stepwise addition of generations around the core followed by removal of protecting groups. For example, PAMAM-NH₂ dendrimers are first synthesized by coupling N-(2-aminoethyl) acryl amide monomers to an ammonia core.

In other embodiments, dendrimers are prepared using convergent methods, in which dendrimers are built from small molecules that end up at the surface of the sphere, and reactions proceed inward building inward and are eventually attached to a core.

Many other synthetic pathways exist for the preparation of dendrimers, such as the orthogonal approach, accelerated approaches, the Double-stage convergent method or the hypercore approach, the hypermonomer method or the branched monomer approach, the Double exponential method; the Orthogonal coupling method or the two-step approach, the two monomers approach, AB₂-CD₂ approach.

In some embodiments, the core of the dendrimer, one or more branching units, one or more linkers/spacers, and/or one or more surface groups can be modified to allow conjugation to further functional groups (branching units, linkers/spacers, surface groups, etc.), monomers, and/or active agents via click chemistry, employing one or more Copper-Assisted Azide-Alkyne Cycloaddition (CuAAC), Diels-Alder reaction, thiol-ene and thiol-yne reactions, and azide-alkyne reactions (Arseneault M et al., Molecules. 2015 May 20; 20(5):9263-94). In some embodiments, pre-made dendrons are clicked onto high-density hydroxyl polymers. ‘Click chemistry’ involves, for example, the coupling of two different moieties (e.g., a core group and a branching unit; or a branching unit and a surface group) via a 1,3-dipolar cycloaddition reaction between an alkyne moiety (or equivalent thereof) on the surface of the first moiety and an azide moiety (e.g., present on a triazine composition or equivalent thereof), or any active end group such as, for example, a primary amine end group, a hydroxyl end group, a carboxylic acid end group, a thiol end group, etc.) on the second moiety.

In some embodiments, dendrimer synthesis replies upon one or more reactions such as thiol-ene click reactions, thiol-yne click reactions, CuAAC, Diels-Alder click reactions, azide-alkyne click reactions, Michael Addition, epoxy opening, esterification, silane chemistry, and a combination thereof.

Any existing dendritic platforms can be used to make dendrimers of desired functionalities, i.e., with a high-density of surface hydroxyl groups by conjugating high-hydroxyl containing moieties such as 1-thio-glycerol or pentaerythritol. Exemplary dendritic platforms such as polyamidoamine (PAMAM), poly (propylene imine) (PPI), poly-L-lysine, melamine, poly (etherhydroxylamine) (PEHAM), poly (esteramine) (PEA) and polyglycerol can be synthesized and explored.

Dendrimers also can be prepared by combining two or more dendrons. Dendrons are wedge-shaped sections of dendrimers with reactive focal point functional groups. Many dendron scaffolds are commercially available. They come in 1, 2, 3, 4, 5, and 6th generations with, respectively, 2, 4, 8, 16, 32, and 64 reactive groups. In certain embodiments, one type of active agents are linked to one type of dendron and a different type of active agent is linked to another type of dendron. The two dendrons are then connected to form a dendrimer. The two dendrons can be linked via click chemistry i.e., a 1,3-dipolar cycloaddition reaction between an azide moiety on one dendron and alkyne moiety on another to form a triazole linker.

Exemplary methods of making dendrimers are described in detail in WO2009/046446, WO2015168347, WO2016025745, WO2016025741, WO2019094952, and U.S. Pat. No. 8,889,101.

B. Dendrimer Complexes

Dendrimer complexes can be formed of therapeutically active agents or compounds conjugated or attached to a dendrimer, a dendritic polymer or a hyperbranched polymer. Conjugation of one or more active agents to a dendrimer are known in the art, and are described in detail in US 2011/0034422, US 2012/0003155, and US 2013/0136697.

In some embodiments, one or more active agents are covalently attached to the dendrimers. In some embodiments, the active agents are attached to the dendrimer via a linking moiety that is designed to be cleaved in vivo. The linking moiety can be designed to be cleaved hydrolytically, enzymatically, or combinations thereof, so as to provide for the sustained release of the active agents in vivo. Both the composition of the linking moiety and its point of attachment to the active agent, are selected so that cleavage of the linking moiety releases either an active agent, or a suitable prodrug thereof. The composition of the linking moiety can also be selected in view of the desired release rate of the active agents. In some embodiments, the functionalized active agents and/or linking moieties are designed to be cleaved at a minimal or insignificant rate in vivo. In preferred embodiments, one or more active agents are functionalized to be non-cleavable or minimally cleavable from the dendrimer-triantennary GalNAc in vivo, for example via one or more amide or ether linkages, optionally, with one or more spacers/linkers.

In some embodiments, the attachment occurs via one or more of disulfide, ester, ether, thioester, carbamate, carbonate, hydrazine, or amide linkages. In preferred embodiments, the attachment occurs via an appropriate spacer that provides an ester bond or an amide bond between the agent and the dendrimer depending on the desired release kinetics of the active agent. In some cases, an ester bond is introduced for releasable form of active agents. In other cases, an amide and/or an ether bond is introduced for non-releasable form of active agents.

Linking moieties generally include one or more organic functional groups. Examples of suitable organic functional groups include secondary amides (—CONH—), tertiary amides (—CONR—), sulfonamide (—S(O)₂—NR—), secondary carbamates (—OCONH—; —NHCOO—), tertiary carbamates (—OCONR—; —NRCOO—), carbonate (—O—C(O)—O—), ureas (—NHCONH—; —NRCONH—; —NHCONR—, —NRCONR—), carbinols (—CHOH—, —CROH—), disulfide groups, hydrazones, hydrazides, ethers (—O—), and esters (—COO—, —CH₂O₂C—, CHRO₂C—), wherein R is an alkyl group, an aryl group, or a heterocyclic group. In general, the identity of the one or more organic functional groups within the linking moiety can be chosen in view of the desired release rate of the active agents. In addition, the one or more organic functional groups can be chosen to facilitate the covalent attachment of the active agents to the dendrimers. In preferred embodiments, the attachment can occur via an appropriate spacer that provides a disulfide bridge between the agent and the dendrimer. The dendrimer complexes are capable of rapid release of the agent in vivo by thiol exchange reactions, under the reduced conditions found in body.

In certain embodiments, the linking moiety includes one or more of the organic functional groups described above in combination with a spacer group. The spacer group can be composed of any assembly of atoms, including oligomeric and polymeric chains; however, the total number of atoms in the spacer group is preferably between 3 and 200 atoms, more preferably between 3 and 150 atoms, more preferably between 3 and 100 atoms, most preferably between 3 and 50 atoms. Examples of suitable spacer groups include alkyl groups, heteroalkyl groups, alkylaryl groups, oligo- and polyethylene glycol chains, and oligo- and poly(amino acid) chains. Variation of the spacer group provides additional control over the release of the active agents in vivo. In embodiments where the linking moiety includes a spacer group, one or more organic functional groups will generally be used to connect the spacer group to both the active agent and the dendrimers.

Reactions and strategies useful for the covalent attachment of active agents to dendrimers are known in the art. See, for example, March, “Advanced Organic Chemistry,” 5th Edition, 2001, Wiley-Interscience Publication, New York) and Hermanson, “Bioconjugate Techniques,” 1996, Elsevier Academic Press, U.S.A. Appropriate methods for the covalent attachment of a given active agent can be selected in view of the linking moiety desired, as well as the structure of the active agents and dendrimers as a whole as it relates to compatibility of functional groups, protecting group strategies, and the presence of labile bonds.

The optimal drug loading will necessarily depend on many factors, including the choice of drug, dendrimer structure and size, and tissues to be treated. In some embodiments, the one or more active drugs are encapsulated, associated, and/or conjugated to the dendrimer at a concentration of about 0.01% to about 45%, preferably about 0.1% to about 30%, about 0.1% to about 20%, about 0.1% to about 10%, about 1% to about 10%, about 1% to about 5%, about 3% to about 20% by weight, and about 3% to about 10% by weight. However, optimal drug loading for any given drug, dendrimer, and site of target can be identified by routine methods, such as those described.

In some embodiments, conjugation of active agents and/or linkers occurs through one or more surface and/or interior groups. Thus, in some embodiments, the conjugation of active agents/linkers occurs via about 1%, 2%, 3%, 4%, or 5% of the total available surface functional groups, preferably hydroxyl groups, of the dendrimers prior to the conjugation. In other embodiments, the conjugation of active agents/linkers occurs on less than 5%, less than 10%, less than 15%, less than 20%, less than 25%, less than 30%, less than 35%, less than 40%, less than 45%, less than 50%, less than 55%, less than 60%, less than 65%, less than 70%, less than 75% total available surface functional groups of the dendrimers prior to the conjugation. In preferred embodiments, dendrimer complexes retain an effective amount of surface functional groups for targeting to specific cell types, whilst conjugated to an effective amount of active agents for treat, prevent, and/or image the disease or disorder.

### V. Methods of Use

Methods of using the dendrimer complex compositions are described. In some embodiments, the dendrimer complexes are used to treat cancer. In other embodiments, the dendrimer complexes are used to treat autoimmune diseases. The methods typically include administering to a subject in a need thereof an effective amount of a composition including dendrimer and one or more active agents to modulate the immune microenvironment, either to decrease an autoimmune response or increase and anti-tumor response.

Methods for modulating immune microenvironment for a desirable immunological outcome are described. In some embodiments, treatment using the compositions reduces or inhibits the number or activity of pro-inflammatory activities of one or more cell types in a disease or disorder associated with excessive pro-inflammatory environment such as in an autoimmune disease. In other embodiments, treating using the compositions reduces or inhibits the number or activity of anti-inflammatory activities of one or more cell types in a disease or disorder associated with excessive immunosuppressive environment such as in cancer cells/tissues.

Methods for enhancing tumor immunogenicity and/or inducing an anti-tumor immune response are described. In some embodiments, treatment using the compositions reduces or inhibits the number or activity of tumor-permissive and immunosuppressive immune cells, for example, TAMs and MDSCs, relative to the number or activity of the tumor-permissive and immunosuppressive immune cells prior to administration of the dendrimer complexes, or compared to administration of the active agent absent a dendrimer scaffold.

Methods of depleting, inhibiting or reducing tumor associated macrophages (TAMs, or M2-like macrophages) in a subject, for example, via blocking proliferation, migration, or activation of the TAMs are described. The methods include administering to the subject the dendrimer complexes including one or more active agents in an effective amount to deplete, inhibit or reduce TAMs. In some embodiments, the compositions are administered in an amount effective to inhibit or reduce the immune suppressive functions of TAM, for example, by decreasing one or more immune suppressive or anti-inflammatory cytokines such as IL-4, IL-10 and IL-13, increasing one or more immune stimulatory cytokines such as IL-12, IL-6, IL-1b, CXCL9, CXCL10, TNFα, or combinations thereof.

Methods of treating cancer mediated or regulated by TAMs are also described. The methods include administering to the subject the dendrimer complexes including one or more active agents in an effective amount to treat and/or alleviate one or more symptoms associated with cancer.

Methods of inducing or increasing the expansion and/or function of pro-inflammatory and tumoricidal classically activated or M1 macrophages are also described.

Myeloid-derived suppressor cells (MDSCs) have emerged as major regulators of immune responses in cancer and other pathological conditions. Two major subsets based on their phenotypic and morphological features: polymorphonuclear (PMN) and monocytic (M)-MDSC. PMN-MDSC is also known as granulocytic MDSC (gMDSC). Phenotypic markers are known for PMN-MDSC (CD11b⁺Ly6G⁺Ly6C^{lo}) and M-MDSC (CD11b⁺Ly6G⁻Ly6C^{hi}). In human peripheral blood mononuclear cell (PBMC), the equivalent to PMN-MDSC are defined as CD11b+CD14−CD15+ or CD11b+CD14−CD66b+ and M-MDSC as CD11b+CD14+HLA−DR^{-/lo}CD15−. CD33 myeloid marker can be used instead of CD11b since very few CD15+ cells are CD11b−. While M-MDSC express the myeloid marker CD33, PMN-MDSC display CD33^{dim} staining (Bronte V et al., Nature Communications 7, Article number: 12150 (2016)). Phenotypically, TAM can be distinguished from M-MDSCs by increased relative expression of F4/80, low-to-intermediate expression of Ly6C and low or undetectable expression of S100A9 protein.

Immune suppression is a main feature of MDSC. Although MDSC were implicated in suppression of different cells of the immune system, the main targets of MDSC are T cells. The main factors implicated in MDSC-mediated immune suppression include arginase (ARG1), iNOS, TGFβ, IL-10, COX2, indoleamine 2,3-dioxygenase (IDO) sequestration of cysteine, decrease of L-selectin expression by T-cells and many others.

Methods of depleting, inhibiting, or reducing MDSCs at tumor tissues in a subject, for example by blocking proliferation, migration, or activation, and/or reversing immuno-suppressive function of the MDSCs, are described. The methods include administering to the subject the dendrimer complexes including one or more active agents in an effective amount to deplete, inhibit, or reduce activity, quantity, and/or function of MDSCs at tumor tissues. Targeting the TRAIL receptor could be a potent and selective method of MDSC depletion (Condamine T, et al. J Clin Invest. (2014); 124:2626-39). Peptibodies including S100A9-derived peptides conjugated to antibody Fc fragments have shown potential in eliminating MDSC in mouse models (Qin H, et al., Nat Med. (2014); 20(6):676-81). Other agents targeting MDSCs include PDE-5 inhibitor tadalafil, Synthetic triterpenoid, nitroaspirin, Class I HDAC inhibitor entinostat, all-trans-retinoic acid (ATRA), gemcitabine, and 5-fluorouracil. Thus, in some embodiments, dendrimers are conjugated to one or more of the agents effective in depleting, inhibiting, or reducing MDSCs. In some embodiments, the compositions are administered in an amount effective to inhibit or reduce the immune suppressive functions of MDSCs, for example, by decreasing one or more of arginase (ARG1) production, iNOS, TGFβ, IL-10, COX2, indoleamine 2,3-dioxygenase (IDO) sequestration of cysteine, or combinations thereof.

Methods for activating one or more innate immune sensors and/or recruitment and activation of Batf3 DCs are also described. Exemplary innate immune sensors include STING pathway for detecting cytosolic DNA sensing. In some embodiments, the compositions are administered in an amount effective to activate one or more innate immune sensors and/or recruitment and activation of Batf3 DCs, to increase the secretion of type I IFNs, CXCL9, and/or CXCL10 by APCs (antigen presenting cells). In some embodiments, the compositions are administered in an amount effective to induce tumor infiltrating lymphocytes (TILs) with increased expression of multiple chemokines capable of recruiting effector T cells, including CCL2, CCL3, CCL4, CCL5, CXCL9, and CXCL10.

In some embodiments, the compositions are administered in an amount effective to induce, cause or stimulate tumor-specific T cells, e.g., cytotoxic CD8+T cells, to have a sustained or amplified biological function, or renew or reactivate exhausted or inactive tumor-specific T cells, or to increase secretion of Granzyme B and/or IFN-γ from cytotoxic CD8+ T-cells, increase proliferation, increase antigen responsiveness (e.g., tumor) relative to such levels before the treatment. In some embodiments, treatment using the compositions leads to a decrease in expression of a regulator of immune suppression (or suppressor of immune activation) such as PD-1, CTLA4, or a combination thereof. In preferred embodiments, the compositions are administered to an amount effective to increase tumor-specific T cells by 10%, 20%, 30%, 40%, 50%, 60%, 70%, 80%, 90%, 100%, 150%, 200%, 250%, 300%, or more than 300% relative to such levels before treatment with the dendrimer compositions.

Methods for treating or ameliorating one or more symptoms of inflammatory or autoimmune diseases are described. In some embodiments, the compositions are used in an amount effective for decreasing production of pro-inflammatory cytokines, and/or promoting generation of immunosuppressive cytokines, and/or immunosuppressive phenotype of one or more immune cell types. In other embodiments, the compositions are used to suppress pro-inflammatory and promote immunosuppressive properties of one or more immune cells involved in the one or more immunological conditions to be treated.

Methods for depleting, inhibiting or reducing pro-inflammatory M1 macrophages or classically activated macrophages (M1-like macrophages) in a subject, for example, by blocking proliferation, migration, or activation of the pro-inflammatory M1 macrophages, are described. The methods include administering to the subject the dendrimer complexes including one or more active agents an effective amount to deplete, inhibit, or reduce the number or activities of the pro-inflammatory M1 macrophages.

In some embodiments, the compositions are administered in an amount effective to inhibit or reduce the immune suppressive functions of pro-inflammatory M1 macrophages, for example, by decreasing one or more pro-inflammatory cytokines such as TNF-α, IL-6, IL-12 and IL-23, chemokines such as CCL-5, CXCL9, CXCL10 and CXCL5, by reducing the recruitment of Th1 and Natural killer (NK) cells.

In some embodiments, the compositions and formulations are used for modulating an immune response in a subject in need thereof by administering an effective amount of the compositions to reduce activation, proliferation and/or generation of one or more pro-inflammatory cells, and/or enhance activation, proliferation and/or generation of one or more suppressive immune cells are provided. In some embodiments, the pro-inflammatory cells are pro-inflammatory M1 macrophages. In further embodiments, the suppressive immune cells are M2-like macrophages. Thus, in some embodiments, the compositions can promote the switch from a pro-inflammatory phenotype (M1 macrophage) to an anti-inflammatory state (M2 macrophage) at one or more diseased tissues/organs of an autoimmune disease by, for example, reducing activation, proliferation and/or generation of M1 macrophage, to enhance activation, proliferation and/or generation of M2 macrophages, and/or to increase the ratio of M2 macrophages to M1 macrophages, effective to ameliorate one or more symptoms of an autoimmune disease.

In some embodiments, the compositions are administered in an amount effective to induce a state of anergy or immune tolerance by increasing the total number or proliferation of regulatory T cells (such as Treg), or reducing the total number or proliferation of the pro-inflammatory T cells (such as Th1 and Th17), or increase the ratio of the level of regulatory T cells (such as Treg) to pro-inflammatory T cells (such as Th1 and Th17). Thus, in some aspects, the compositions are formulated for inducing anergy or tolerance by increasing Treg levels, or decrease pro-inflammatory T cell levels, or both. In other embodiments, the compositions can promote suppressor/regulatory cells to cause anergy or clonal deletion of T cells by secreting inhibitory cytokines or inducing T cell apoptosis in the periphery.

In further embodiments, the compositions can attenuate production of inflammatory cytokines and/or induce the production of anti-inflammatory cytokines. Exemplary inflammatory cytokines include TNF-α, IL-1, IL-6, IL-12, IL-17, IL21, and IL23.

A. Treatment Regimen

1. Dosage and Effective Amounts

Dosage and dosing regimens are dependent on the severity and location of the disorder or injury and/or methods of administration, and are known to those skilled in the art. A therapeutically effective amount of the dendrimer composition used in the treatment of cancer or autoimmune diseases is typically sufficient to reduce or alleviate one or more symptoms of cancer or autoimmune diseases.

Symptoms of cancer may be physical, such as tumor burden, or biological such as proliferation of cancer cells. Accordingly, the amount of dendrimer complex can be effective to, for example, kill tumor cells or inhibit proliferation or metastasis of the tumor cells. Preferably the dendrimer composition including one or more active agents, for example immunomodulatory agents, are preferentially delivered to cells in and around tumor tissues, for example, cancerous cells or immune cells associated with tumor tissues (e.g. M2 macrophages). Preferably the active agents do not target or otherwise modulate the activity or quantity of healthy cells not within or associated with tumor tissues, or do so at a reduced level compared to cancer or cancer-associated cells. In this way, by-products and other side effects associated with the compositions are reduced, preferably leading directly or indirectly to cancer cell death. In some embodiments, the active agent directly or indirectly reduces cancer cell migration, angiogenesis, immune escape, radioresistance, or a combination thereof. In some embodiments, the active agent directly or indirectly induces a change in the cancer cell itself or its microenvironment that reduces suppression or induces activation of an immune response against the cancer cells. For example, in some embodiments, the composition is administered in an effective amount to enhance and/or prolong the activation, proliferation, and/or function of T cells (i.e., increasing tumor-specific proliferation of T cells, enhance cytokine production by T cells, stimulate differentiation, stimulate effector functions of T cells and/or promote T cell survival) or overcome T cell exhaustion and/or anergy.

In some in vivo approaches, the dendrimer complexes are administered to a subject in a therapeutically effective amount to reduce tumor size. In some embodiments, an effective amount of the composition is used to put cancer in remission and/or keep the cancer in remission. Also provided are effective amounts of the compositions to reduce or stop cancer stem cell proliferation.

The actual effective amounts of dendrimer complex can vary according to factors including the specific active agent administered, the particular composition formulated, the mode of administration, and the age, weight, condition of the subject being treated, as well as the route of administration and the disease or disorder. The subjects are typically mammals, most preferably, humans. Generally, for intravenous injection or infusion, the dosage may be lower.

In general, the timing and frequency of administration will be adjusted to balance the efficacy of a given treatment or diagnostic schedule with the side-effects of the given delivery system. Exemplary dosing frequencies include continuous infusion, single and multiple administrations such as hourly, daily, weekly, monthly or yearly dosing.

In some embodiments, dosages are administered once, twice, or three times daily, or every other day, two days, three days, four days, five days, or six days to a human. In some embodiments, dosages are administered about once or twice every week, every two weeks, every three weeks, or every four weeks. In some embodiments, dosages are administered about once or twice every month, every two months, every three months, every four months, every five months, or every six months.

When administered parenterally, the dose administered may range from 0.1 to 100 mg/kg of body weight. Higher doses may be given initially to load the patient with drug and maximize uptake in the diseased tissues (e.g. tumor). After the loading dose, patients may receive a maintenance dose. Loading doses may range from 10 to 100 mg/kg of body weight and maintenance doses may range from 0.1 to <10 mg/kg of body weight. When administered enterally or topically, the dose required for treatment may be up to 10 fold greater than the effective parenteral dose. The optimal dose is selected from the safety and efficacy results of each tested dose for each drug in patients.

It will be understood by those of ordinary skill that a dosing regimen can be any length of time sufficient to treat the disorder in the subject. In some embodiments, the regimen includes one or more cycles of a round of therapy followed by a drug holiday (e.g., no drug). The drug holiday can be 1, 2, 3, 4, 5, 6, or 7 days; or 1, 2, 3, 4 weeks, or 1, 2, 3, 4, 5, or 6 months.

2. Controls

The therapeutic result of the dendrimer complex compositions including one or more active agents can be compared to a control. Suitable controls are known in the art and include, for example, untreated cells or an untreated subject. A typical control is a comparison of a condition or symptom of a subject prior to and after administration of the targeted agent. The condition or symptom can be a biochemical, molecular, physiological, or pathological readout. For example, the effect of the composition on a particular symptom, pharmacologic, or physiologic indicator can be compared to an untreated subject, or the condition of the subject prior to treatment. In some embodiments, the symptom, pharmacologic, or physiologic indicator is measured in a subject prior to treatment, and again one or more times after treatment is initiated. In some embodiments, the control is a reference level, or average determined based on measuring the symptom, pharmacologic, or physiologic indicator in one or more subjects that do not have the disease or condition to be treated (e.g., healthy subjects). In some embodiments, the effect of the treatment is compared to a conventional treatment that is known the art.

B. Combination Therapies and Procedures

In some embodiments, compositions of dendrimers conjugated or complexed with one or more immunomodulatory agents and/or additional therapeutic or diagnostic agents are administered in combination with one or more conventional therapies, for example, a conventional cancer therapy. In some embodiments, the conventional therapy includes administration of one or more of the compositions in combination with one or more additional active agents. The combination therapies can include administration of the active agents together in the same admixture, or in separate admixtures. Therefore, in some embodiments, the pharmaceutical composition includes two, three, or more active agents. Such formulations typically include an effective amount of an immunomodulatory agent targeting tumor microenvironment. The additional active agent(s) can have the same, or different mechanisms of action. In some embodiments, the combination results in an additive effect on the treatment of the cancer. In some embodiments, the combinations result in a more than additive effect on the treatment of the disease or disorder.

In some embodiments, the formulation is formulated for intravenous, subcutaneous, or intramuscular administration to the subject, or for enteral administration. In some embodiments, the formulation is administered prior to, in conjunction with, subsequent to, or in alternation with treatment with one or more additional therapies or procedures. In some embodiments the additional therapy is performed between drug cycles or during a drug holiday that is part of the compositions dosage regime. For example, in some embodiments, the additional therapy or procedure is surgery, a radiation therapy, or chemotherapy.

Additional therapeutic agents include conventional cancer therapeutics such as chemotherapeutic agents, cytokines, chemokines, and radiation therapy. The majority of chemotherapeutic drugs can be divided into alkylating agents, antimetabolites, anthracyclines, plant alkaloids, topoisomerase inhibitors, and other antitumour agents. These drugs affect cell division or DNA synthesis and function in some way. Additional therapeutics include monoclonal antibodies and the tyrosine kinase inhibitors e.g., imatinib mesylate (GLEEVEC® or GLIVEC®), which directly targets a molecular abnormality in certain types of cancer (chronic myelogenous leukemia, gastrointestinal stromal tumors).

Representative chemotherapeutic agents include, but are not limited to, amsacrine, bleomycin, busulfan, camptothecin, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epipodophyllotoxins, epirubicin, etoposide, etoposide phosphate, fludarabine, fluorouracil, gemcitabine, hydroxycarb amide, idarubicin, ifosfamide, innotecan, leucovorin, liposomal doxorubicin, liposomal daunorubici, lomustine, mechlorethamine, melphalan, mercaptopurine, mesna, methotrexate, mitomycin, mitoxantrone, oxaliplatin, paclitaxel, pemetrexed, pentostatin, procarbazine, raltitrexed, satraplatin, streptozocin, teniposide, tegafur-uracil, temozolomide, teniposide, thiotepa, tioguanine, topotecan, treosulfan, vinblastine, vincristine, vindesine, vinorelbine, vorinostat, taxol, trichostatin A and derivatives thereof, trastuzumab (HERCEPTIN®), cetuximab, and rituximab (RITUXAN® or MABTHERA®), bevacizumab (AVASTIN®), and combinations thereof. Representative pro-apoptotic agents include, but are not limited to, fludarabinetaurosporine, cycloheximide, actinomycin D, lactosylceramide, 15d-PGJ(2)5 and combinations thereof.

In some embodiments, the compositions and methods are used prior to or in conjunction with an immunotherapy such inhibition of checkpoint proteins such as components of the PD-1/PD-L1 axis or CD28-CTLA-4 axis using one or more immune checkpoint modulators (e.g., PD-1 antagonists, PD-1 ligand antagonists, and CTLA4 antagonists), adoptive T cell therapy, and/or a cancer vaccine. Exemplary immune checkpoint modulators used in immunotherapy include Pembrolizumab (anti-PD1 mAb), Durvalumab (anti-PDL1 mAb), PDR001 (anti-PD1 mAb), Atezolizumab (anti-PDL1 mAb), Nivolumab (anti-PD1 mAb), Tremelimumab (anti-CTLA4 mAb), Avelumab (anti-PDL1 mAb), and RG7876 (CD40 agonist mAb).

Methods of adoptive T cell therapy are known in the art and used in clinical practice. Generally adoptive T cell therapy involves the isolation and ex vivo expansion of tumor specific T cells to achieve greater number of T cells than what could be obtained by vaccination alone. The tumor specific T cells are then infused into patients with cancer in an attempt to give their immune system the ability to overwhelm remaining tumor via T cells, which can attack and kill the cancer. Several forms of adoptive T cell therapy can be used for cancer treatment including, but not limited to, culturing tumor infiltrating lymphocytes or TIL; isolating and expanding one particular T cell or clone; and using T cells that have been engineered to recognize and attack tumors. In some embodiments, the T cells are taken directly from the patient's blood. Methods of priming and activating T cells in vitro for adaptive T cell cancer therapy are known in the art. See, for example, Wang, et al, Blood, 109(11):4865-4872 (2007) and Hervas-Stubbs, et al, J. Immunol., 189(7):3299-310 (2012).

Historically, adoptive T cell therapy strategies have largely focused on the infusion of tumor antigen specific cytotoxic T cells (CTL) which can directly kill tumor cells. However, CD4+ T helper (Th) cells such as Th1, Th2, Tfh, Treg, and Th17 can also be used. Th can activate antigen-specific effector cells and recruit cells of the innate immune system such as macrophages and dendritic cells to assist in antigen presentation (APC), and antigen primed Th cells can directly activate tumor antigen-specific CTL. As a result of activating APC, antigen specific Th₁ have been implicated as the initiators of epitope or determinant spreading which is a broadening of immunity to other antigens in the tumor. The ability to elicit epitope spreading broadens the immune response to many potential antigens in the tumor and can lead to more efficient tumor cell kill due to the ability to mount a heterogeneic response. In this way, adoptive T cell therapy can used to stimulate endogenous immunity.

In some embodiments, the T cells express a chimeric antigen receptor (CARs, CAR T cells, or CARTs). Artificial T cell receptors are engineered receptors, which graft a particular specificity onto an immune effector cell. Typically, these receptors are used to graft the specificity of a monoclonal antibody onto a T cell and can be engineered to target virtually any tumor associated antigen. First generation CARs typically had the intracellular domain from the CD3 ζ-chain, which is the primary transmitter of signals from endogenous TCRs. Second generation CARs add intracellular signaling domains from various costimulatory protein receptors (e.g., CD28, 41BB, ICOS) to the cytoplasmic tail of the CAR to provide additional signals to the T cell, and third generation CARs combine multiple signaling domains, such as CD3z-CD28-41BB or CD3z-CD28-OX40, to further enhance effectiveness.

In some embodiments, the compositions and methods are used prior to or in conjunction with a cancer vaccine, for example, a dendritic cell cancer vaccine. Vaccination typically includes administering a subject an antigen (e.g., a cancer antigen) together with an adjuvant to elicit therapeutic T cells in vivo. In some embodiments, the cancer vaccine is a dendritic cell cancer vaccine in which the antigen delivered by dendritic cells primed ex vivo to present the cancer antigen. Examples include PROVENGE® (sipuleucel-T), which is a dendritic cell-based vaccine for the treatment of prostate cancer (Ledford, et al., Nature, 519, 17-18 (5 Mar. 2015). Such vaccines and other compositions and methods for immunotherapy are reviewed in Palucka, et al., Nature Reviews Cancer, 12, 265-277 (April 2012).

In some embodiments, the compositions and methods are used prior to or in conjunction with surgical removal of tumors, for example, in preventing primary tumor metastasis. In some embodiments, the compositions and methods are used to enhance body's own anti-tumor immune functions.

C. Subjects to be Treated

In general, the compositions and methods of treatment thereof are useful in the context of cancer, including tumor therapy. The compositions can also be used for treatment of other diseases, disorders and injury including inflammatory diseases, including, but not limited to, ulcerative colitis, Crohn's disease, and rheumatoid arthritis.

In some embodiments, the subject to be treated is a human. All the methods described can include the step of identifying and selecting a subject in need of treatment, or a subject who would benefit from administration with the compositions. Therefore, in some embodiments, compositions of dendrimers conjugated or complexed with one or more immunomodulatory agents and/or additional therapeutic or diagnostic agents are administered to a subject in need of immunomodulation in the context of treatment for cancer, or treatment of other diseases, disorders and injury including inflammatory diseases such as ulcerative colitis, Crohn's disease, rheumatoid arthritis, and bone diseases.

1. Cancer

In some embodiments, compositions of dendrimers conjugated or complexed with one or more immunomodulatory agents and/or additional therapeutic or diagnostic agents are administered to a subject having a proliferative disease, such as a benign or malignant tumor. In some embodiments, the subjects to be treated have been diagnosed with stage I, stage II, stage III, or stage IV cancer.

The term cancer refers specifically to a malignant tumor. In addition to uncontrolled growth, malignant tumors exhibit metastasis. In this process, small clusters of cancerous cells dislodge from a tumor, invade the blood or lymphatic vessels, and are carried to other tissues, where they continue to proliferate. In this way a primary tumor at one site can give rise to a secondary tumor at another site.

The compositions and methods are useful for treating subjects having benign or malignant tumors by delaying or inhibiting the growth of a tumor in a subject, reducing the growth or size of the tumor, inhibiting or reducing metastasis of the tumor, and/or inhibiting or reducing symptoms associated with tumor development or growth.

Malignant tumors which may be treated are classified according to the embryonic origin of the tissue from which the tumor is derived. Carcinomas are tumors arising from endodermal or ectodermal tissues such as skin or the epithelial lining of internal organs and glands. The compositions are particularly effective in treating carcinomas. Sarcomas, which arise less frequently, are derived from mesodermal connective tissues such as bone, fat, and cartilage. The leukemias and lymphomas are malignant tumors of hematopoietic ceils of the bone marrow. Leukemias proliferate as single cells, whereas lymphomas tend to grow as tumor masses. Malignant tumors may show up at numerous organs or tissues of the body to establish a cancer.

The types of cancer that can be treated with the compositions and methods include, but are not limited to, cancers such as vascular cancer such as multiple myeloma, adenocarcinomas and sarcomas, of bone, bladder, brain, breast, cervical, colorectal, esophageal, kidney, liver, lung, nasopharangeal, pancreatic, prostate, skin, stomach, and uterine. In some embodiments, the compositions are used to treat multiple cancer types concurrently. The compositions can also be used to treat metastases or tumors at multiple locations.

Exemplary tumor cells include tumor cells of cancers, including leukemias including, but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as, but not limited to, chronic myelocytic (granulocytic) leukemia, chronic lymphocytic leukemia, hairy cell leukemia; polycythemia vera; lymphomas such as, but not limited to, Hodgkin's disease, non-Hodgkin's disease; multiple myelomas such as, but not limited to, smoldering multiple myeloma, nonsecretory myeloma, osteosclerotic myeloma, plasma cell leukemia, solitary plasmacytoma and extramedullary plasmacytoma; Waldenström's macroglobulinemia; monoclonal gammopathy of undetermined significance; benign monoclonal gammopathy; heavy chain disease; bone and connective tissue sarcomas such as, but not limited to, bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors including, but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including, but not limited to, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer, including, but not limited to, pheochromocytom and adrenocortical carcinoma; thyroid cancer such as but not limited to papillary or follicular thyroid cancer, medullary thyroid cancer and anaplastic thyroid cancer; pancreatic cancer, including, but not limited to, insulinoma, gastrinoma, glucagonoma, vipoma, somatostatin-secreting tumor, and carcinoid or islet cell tumor; pituitary cancers including, but not limited to, Cushing's disease, prolactin-secreting tumor, acromegaly, and diabetes insipius; eye cancers including, but not limited to, ocular melanoma such as iris melanoma, choroidal melanoma, and cilliary body melanoma, and retinoblastoma; vaginal cancers, including, but not limited to, squamous cell carcinoma, adenocarcinoma, and melanoma; vulvar cancer, including, but not limited to, squamous cell carcinoma, melanoma, adenocarcinoma, basal cell carcinoma, sarcoma, and Paget's disease; cervical cancers including, but not limited to, squamous cell carcinoma, and adenocarcinoma; uterine cancers including, but not limited to, endometrial carcinoma and uterine sarcoma; ovarian cancers including, but not limited to, ovarian epithelial carcinoma, borderline tumor, germ cell tumor, and stromal tumor; esophageal cancers including, but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers including, but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers including, but not limited to, hepatocellular carcinoma and hepatoblastoma, gallbladder cancers including, but not limited to, adenocarcinoma; cholangiocarcinomas including, but not limited to, papillary, nodular, and diffuse; lung cancers including, but not limited to, non-small cell lung cancer, squamous cell carcinoma (epidermoid carcinoma), adenocarcinoma, large-cell carcinoma and small-cell lung cancer; testicular cancers including, but not limited to, germinal tumor, seminoma, anaplastic, classic (typical), spermatocytic, nonseminoma, embryonal carcinoma, teratoma carcinoma, choriocarcinoma (yolk-sac tumor), prostate cancers including, but not limited to, adenocarcinoma, leiomyosarcoma, and rhabdomyosarcoma; penal cancers; oral cancers including, but not limited to, squamous cell carcinoma; basal cancers; salivary gland cancers including, but not limited to, adenocarcinoma, mucoepidermoid carcinoma, and adenoidcystic carcinoma; pharynx cancers including, but not limited to, squamous cell cancer, and verrucous; skin cancers including, but not limited to, basal cell carcinoma, squamous cell carcinoma and melanoma, superficial spreading melanoma, nodular melanoma, lentigo malignant melanoma, acral lentiginous melanoma; kidney cancers including, but not limited to, renal cell cancer, adenocarcinoma, hypernephroma, fibrosarcoma, transitional cell cancer (renal pelvis and/or uterer); Wilms' tumor; bladder cancers including, but not limited to, transitional cell carcinoma, squamous cell cancer, adenocarcinoma, carcinosarcoma. In one embodiment, the cancer is brain metastasis in patient with leukemia.

Cancers that can be prevented, treated or otherwise diminished by the compositions include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma, papillary adenocarcinomas, and gastric cancer (for a review of such disorders, see Fishman et al., 1985, Medicine, 2d Ed., J. B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

In some embodiments, the cancers are characterized as being triple negative breast cancer, or having one or more KRAS-mutations, EGFR mutations, ALK mutations, RB1 mutations, HIF mutations, KEAP mutations, NRF mutations, or other metabolic-related mutations, or combinations thereof. The methods and compositions as described are useful for both prophylactic and therapeutic treatment.

Therapeutic treatment involves administering to a subject a therapeutically effective amount of the compositions or pharmaceutically acceptable salts thereof as described after cancer is diagnosed.

In further embodiments, the compositions are used for prophylactic use i.e. prevention, delay in onset, diminution, eradication, or delay in exacerbation of signs or symptoms after onset, and prevention of relapse. For prophylactic use, a therapeutically effective amount of the compounds and compositions or pharmaceutically acceptable salts thereof as described are administered to a subject prior to onset (e.g., before obvious signs of cancer), during early onset (e.g., upon initial signs and symptoms of cancer), or after an established development of cancer. Prophylactic administration can occur for several days to years prior to the manifestation of symptoms. Prophylactic administration can be used, for example, in the chemopreventative treatment of subjects presenting precancerous lesions, those diagnosed with early stage malignancies, and for subgroups with susceptibilities (e.g., family, racial, and/or occupational) to particular cancers.

In some embodiments, the subject to be treated is one with one or more solid tumors. A solid tumor is an abnormal mass of tissue that usually does not contain cysts or liquid areas. Solid tumors may be benign (not cancer), or malignant (cancer). Examples of solid tumors are sarcomas, carcinomas, and lymphomas. In preferred embodiments, the compositions and methods are effective in treating one or more symptoms of cancers of the skin, lung, liver, pancreas, brain, kidney, breast, prostate, colon and rectum, bladder, etc. In further embodiment, the tumor is a focal lymphoma or a follicular lymphoma.

### Renal Cell Cancer (RCC)

In some embodiments, the subject to be treated has renal cell cancer (RCC). Renal cell cancer is a disease in which malignant (cancer) cells form in tubules of the kidney. RCC, also known as renal cell adenocarcinoma, or kidney cancer, is a disease in which malignant cells develop within the lining of tubules in the kidney. Symptoms include blood in the urine (40% of affected persons at diagnosis), flank pain (40%), a mass in the abdomen or flank (25%), weight loss (33%), fever (20%), high blood pressure (20%), night sweats and general malaise, as well as increased abdominal mass/bloating. There are two subtypes: sporadic (i.e., non-hereditary), and hereditary. Renal cell carcinoma (RCC) is not a single entity, but a collection of different tumors, each derived from the various parts of the nephron, and each possessing distinct genetic characteristics, histological features, and/or clinical phenotypes. Metastatic renal cell carcinoma (mRCC) is the spread of the primary renal cell carcinoma from the kidney to other organs. 25-30% of patients with RCC exhibit metastatic spread by the time they are diagnosed, owing largely to the fact that clinical signs are generally mild until RCC progresses to a more severe stage. Common sites for metastasis are the lymph nodes, lung, bones, liver and brain.

Tumor associated macrophages (TAMs) are an important element of tumor stroma. They originate from blood monocytes attracted by chemokines and cytokines produced by tumor cells and, being instructed by tumor microenvironment, develop into potent tumor-supporting cell population. TAMs directly stimulate tumor cell proliferation, promote angiogenesis, provide for efficient immune escape by producing immunosuppressive cytokines and facilitate tumor dissemination by producing extracellular matrix remodeling enzymes. In renal cell carcinoma (RCC), increased density of TAMs is associated with poor survival of patients (see Kovaleva, et al., Anal Cell Pathol (Amst). 2016; 2016: 9307549, the content of which is incorporated by reference in its entirety). Macrophages isolated from RCC tumors were shown to produce pro-inflammatory cytokines TNFα, IL-1β, IL-6, and CCL2, and it may be that RCC is a tumor with hybrid phenotype of TAMs that exhibit properties of both type 1 (M1) macrophages and type 2 (M2) macrophages. Therefore, in some embodiments, the compositions and methods are effective for treating renal cell carcinoma in a subject in need thereof. The subject can be diagnosed as having renal cell cancer, or be identified as being at enhanced risk of renal cell cancer. The compositions and methods are useful for treating subjects having renal cell cancer by delaying or inhibiting the growth of a tumor in a subject, reducing the growth or size of the tumor, inhibiting or reducing metastasis of the tumor, and/or inhibiting or reducing symptoms associated with tumor development or growth. In a particular embodiment, the methods reduce or inhibit one or more immunosuppressive cells at a site of a renal cell cancer tumor in a subject identified as having renal cell cancer, by administering to the subject an effective amount of a pharmaceutical composition including a dendrimer complexed or conjugated with one or more active agents effective in reducing tumor growth in the subject. In some embodiments, the method and chemical characteristics of the attachment between the dendrimer and the active agent impacts the efficiency of the active agent for reducing tumor size. In a preferred embodiment, the active agent(s), is attached to the dendrimer via an ether and/or amide bond. In some embodiments, the active agent(s), is attached to the dendrimer via a linker. In a particular embodiment, the active agent(s) is attached to the dendrimer via a linker that is conjugated to the dendrimer via an ether bond, and the active agent is conjugated to the linker via an amide bond. An exemplary active agent effective for reducing tumor size is sunitinib, or one or more sunitinib analogs. In a preferred embodiment, sunitinib, or one or more sunitinib analogs is attached to the dendrimer via an amide bond.

In some embodiments, the methods include combination therapies with one or more additional active agents to inhibit the growth and spread of renal tumors. Exemplary active agents include Nivolumab, Axitinib, Sunitinib, Cabozantinib, Everolimus, Lenvatinib, Pazopanib, Bevacizumab, Sorafenib, Tivozanib, Temsirolimus, Interleukin-2 (IL-2), Interferon-a, ipilimumab, atezolizumab, varlilumab, durvalumab, avelumab, LAG525, MBG453, TRC105, and savolitinib.

2. Autoimmune or Inflammatory Disease

In some embodiments, compositions of dendrimers conjugated or complexed with one or more immunomodulatory agents and/or additional therapeutic or diagnostic agents are administered to a subject with an autoimmune or inflammatory disease or disorder. Autoimmune disease happens when the body's natural defense system cannot effectively differentiate between the body's own cells and foreign cells, causing the body to mistakenly attack normal cells. There are more than 80 types of autoimmune diseases that affect a wide range of body parts. Common autoimmune diseases include rheumatoid arthritis, psoriasis, psoriatic arthritis, systemic lupus erythematosus (SLE), type 1 diabetes, inflammatory bowel disease, and thyroid diseases.

In some embodiments, the compositions can also be used for treatment of autoimmune or inflammatory disease or disorder such as rheumatoid arthritis, systemic lupus erythematosus, alopecia areata, anklosing spondylitis, antiphospholipid syndrome, autoimmune Addison's disease, autoimmune hemolytic anemia, autoimmune hepatitis, autoimmune inner ear disease, autoimmune lymphoproliferative syndrome (alps), autoimmune thrombocytopenic purpura (ATP), Bechet's disease, bullous pemphigoid, cardiomyopathy, celiac sprue-dermatitis, chronic fatigue syndrome immune deficiency, syndrome (CFIDS), chronic inflammatory demyelinating polyneuropathy, cicatricial pemphigoid, cold agglutinin disease, Crest syndrome, Crohn's disease, Dego's disease, dermatomyositis, dermatomyositis—juvenile, discoid lupus, essential mixed cryoglobulinemia, fibromyalgia—fibromyositis, grave's disease, guillain-barre, hashimoto's thyroiditis, idiopathic pulmonary fibrosis, idiopathic thrombocytopenia purpura (ITP), Iga nephropathy, insulin dependent diabetes (Type I), juvenile arthritis, Meniere's disease, mixed connective tissue disease, multiple sclerosis, myasthenia gravis, pemphigus vulgaris, pernicious anemia, polyarteritis nodosa, polychondritis, polyglancular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, sarcoidosis, scleroderma, Sjogren's syndrome, stiff-man syndrome, Takayasu arteritis, temporal arteritis/giant cell arteritis, ulcerative colitis, uveitis, vasculitis, vitiligo, and Wegener's granulomatosis.

In some embodiments, the compositions and methods can also be used for treatment of autoimmune or inflammatory diseases or disorders involving bones and joints, including infections and immunologically-mediated local and systemic diseases.

### i. Inflammatory Bone Diseases and Disorders

In some embodiments, the subject to be treated is one with one or more inflammatory bone diseases. Inflammatory bone diseases are caused by seemingly unprovoked activation of immune processes, resulting in osseous inflammation and diseases/disorders of the bones. Inflammatory bone lesions can be characterized by chronic inflammatory processes, with little or no histopathology (Stern, et al., Rheum Dis Clin North Am. 2013 November; 39(4): 10.1016/j.rdc.2013.05.002). Therefore, in some embodiments, the compositions and methods are effective for treating one or more inflammatory bone diseases, including osteomyelitis (acute osteomyelitis, sub-acute osteomyelitis, chronic osteomyelitis), chronic non-bacterial osteomyelitis (CNO); SAPHO syndrome; Majeed syndrome; deficiency of interleukin-1 receptor antagonist (DIRA); and cherubism.

In a particular embodiment, the subject to be treated is one with osteomyelitis. Osteomyelitis is inflammation associated with the bone and/or the marrow, which may occur due to bacterial or fungal infection within the bone tissue. Osteomyelitis can develop following infection from the bloodstream, for example, due to injury or surgery, or it can occur in the absence of infection (chronic non-bacterial osteomyelitis), and has historically been difficult to treat. Therefore, in some embodiments, compositions and methods for targeting active agents to inflammatory macrophages are effective for treating osteomyelitis. Exemplary osteomyelitis diseases and disorders that can be treated include chronic non-bacterial osteomyelitis, acute osteomyelitis, sub-acute osteomyelitis, chronic osteomyelitis, or hematogenous osteomyelitis of the leg, spine, arm, jaw, or pelvic bones. The compositions and methods are effective for treating or preventing osteomyelitis in a subject diagnosed with osteomyelitis, or a subject identified as being at increased risk of developing osteomyelitis, such a person with a deep wound, blood infection, bone surgery, compromised immunity, HIV or diabetes. In a preferred embodiment, the subject to be treated is one with auto-inflammatory osteomyelitis (chronic non-bacterial osteomyelitis).

### ii. Inflammatory Arthropathies

In some embodiments, the subject to be treated is one with one or more inflammatory joint diseases. Macrophage-mediated pro-inflammatory mechanisms contribute to synovial inflammation associated with the pathogenesis of many acute and chronic joint diseases. Therefore, in some embodiments, the compositions and methods are effective for treating one or more inflammatory arthropathies. Exemplary inflammatory arthropathies include posttraumatic joint injury, synovitis, arthritis, Lupus erythematosus, ankylosing spondylitis, juvenile ankylosing spondylitis, acute anterior uveitis, fibromyalgia and scleroderma.

In particular embodiments, the subject to be treated is one with arthritis. Exemplary arthritic diseases which can be treated include osteoarthritis, rheumatoid arthritis, juvenile arthritis, Reiter's syndrome, psoriatic arthritis, enteropathic arthropathy, infectious arthritis and reactive arthritis.

### Osteoarthritis

In some embodiments, the subject to be treated is one with osteoarthritis. Osteoarthritis is a family of degenerative diseases with diverse etiology and pathogenesis, affecting multiple joint tissues. Osteoarthritis can affect all joint tissues, and is characterized by progressive degeneration of articular cartilage, neovascular invasion of articular surface, subchondral bone remodeling, osteophyte formation, bone marrow lesions, meniscal damage and synovial inflammation (synovitis). Articular cartilage is at high risk of damage during trauma, or infection, as well as age-related wear and tear. If left untreated, trauma results in lesions in the underlying subchondral bone, leading to degenerated cartilage, joint inflammation/disturbances in the joint as a whole, and ultimately resulting in osteoarthritis. Therapy for osteoarthritis is directed to non-pharmacological treatments, and symptomatic treatment (pain management). Macrophages play a significant role in modulating the severity of osteoarthritis by mediating joint inflammation via various secreted mediators. Synovial inflammation in osteoarthritis is associated with inflammatory chemokines, cytokines, and other inflammatory markers within the synovial fluid (Goldring, et al., Curr Opin Rheumatol., 2011 September; 23(5): 471-478). Macrophages are the most common immune cell type present in the inflamed synovial tissue of patients with osteoarthritis. Therefore, in some embodiments, compositions and methods for targeting active agents to inflammatory macrophages are effective for treating a subject with osteoarthritis. In some embodiments, the methods prevent or reduce synovial inflammation, reduce or prevent inflammatory chemokines, cytokines, and other inflammatory markers associated with osteoarthritis, or increase or induce macrophage-mediated repair and regeneration of cartilage in a patient with osteoarthrirtis

### Rheumatoid Arthritis

In some embodiments, the subject to be treated is one with Rheumatoid Arthritis (RA). Rheumatoid arthritis is a long-term condition that causes swelling and stiffness and pain, in the joints, especially in the hands, feet and wrists. Rheumatoid arthritis is an autoimmune disease, whereby the immune system attacks cells that line the joints, and causing inflammation in the joints. Symptoms include swollen, stiff and painful joints, a low red blood cell count, inflammation around the lungs, inflammation around the heart, fever and low energy may also be present. Over time, the inflammation damages the joints, cartilage and bone. The condition affects non-articular organs in more than 15-25% of cases. RA is a systemic (whole body) autoimmune disease, which has genetic and environmental risk factors. Rheumatoid arthritis is initiated as a state of persistent cellular activation, which leads to autoimmune complexes in joints, and other organs, and macrophages are a central component of the inflammation associated with Rheumatoid arthritis: Fibroblast-like synoviocytes play a key role in development of clinical manifestations, including inflammation of the synovial membrane, and joint/organ damage. Three phases of progression of RA (an initiation phase, due to non-specific inflammation; an amplification phase, due to T cell activation; and a chronic inflammatory phase, with tissue injury resulting from cytokines including IL-1, TNF-alpha and IL-6) lead B lymphocytes to produce rheumatoid factors and ACPA of the IgG and IgM classes in large quantities. These, in turn, activate macrophages through Fc receptor and complement binding, leading to the intense inflammation characteristic of Rheumatoid arthritis. Therefore, in some embodiments, the compositions and methods for targeting active agents to inflammatory macrophages are effective for treating a subject with Rheumatoid arthritis. In some embodiments, the methods prevent or reduce synovial inflammation, reduce or prevent inflammatory chemokines, cytokines, and other inflammatory markers associated with Rheumatoid arthritis, and/or increase or induce macrophage-mediated repair and regeneration of cartilage in a patient with Rheumatoid arthritis.

The present invention will be further understood by reference to the following non-limiting examples.

### EXAMPLES

### Example 1: Dendrimer Distribution in Immune Cells in Tumor Tissue

### Methods and Materials

Mice

Female C57BL/6 mice (C57BL/6 NCrl Charles River) were eight weeks old on Day 1 of the study and had a body weight (BW) range of 17.7 to 21.5 g. Animals were fed ad libitum water (reverse osmosis, 1 ppm Cl) and NIH 31 Modified and Irradiated Lab Diet® including 18.0% crude protein, 5.0% crude fat, and 5.0% crude fiber. The mice were housed on irradiated ENRICH-O'COBS™ bedding in static microisolators on a 12-hour light cycle at 20-22° C. (68-72° F.) and 40-60% humidity. Charles River Discovery Services North Carolina (CR Discovery Services) specifically complies with the recommendations of the Guide for Care and Use of Laboratory Animals with respect to restraint, husbandry, surgical procedures, feed and fluid regulation, and veterinary care. The animal care and use program at CR Discovery Services is accredited by the Association for Assessment and Accreditation of Laboratory Animal Care International (AAALAC), which assures compliance with accepted standards for the care and use of laboratory animals.

Tumor Cell Culture

MC38 murine colon carcinoma cells were grown to mid-log phase in DMEM medium containing 10% fetal bovine serum, 2 mM glutamine, 100 units/mL penicillin G, 100 μg/mL streptomycin sulfate and 25 μg/mL gentamicin. The tumor cells were cultured in tissue culture flasks in a humidified incubator at 37° C., in an atmosphere of 5% CO₂ and 95% air.

In Vivo Implantation

On the day of implant, MC38 cells were harvested during log phase growth and resuspended in phosphate buffered saline (PBS) at a concentration of 5×10⁶ cells/mL. Tumors were initiated by subcutaneously implanting 5×10⁵ MC38 cells (0.1 mL suspension) into the right flank of each test animal and tumors were monitored as their volumes approached the target range of 80 to 120 mm³. Twelve days after tumor implantation, designated as Day 1 of the study, the animals were sorted into four groups (n=10 for Groups 1 through 3 and n=1 for Group 4) with individual tumor volumes ranging from 75 to 126 mm³ and group mean tumor volumes between 95 and 108 mm³. Tumors were measured in two dimensions using calipers, and volume was calculated using the formula:

$$Tumor Volume ( {mm}^{3} ) = \frac{{w}^{2} \times l}{2}$$

where w=width and l=length, in mm, of the tumor. Tumor weight may be estimated with the assumption that 1 mg is equivalent to 1 mm³ of tumor volume.

Therapeutic Agents

Ashvattha Therapeutics, Inc provided D-Cy5 (Lot No. 1, coded FFZ1), stored at −80° C. and protected from the light before and after dosing. On the day of dosing, stock solution of D-Cy5 (a small molecule dye, representative of small molecule drugs) (both 8.25 mg/mL) was equilibrated to room temperature, protected from light and heat. Agents were then sonicated and vortexed for 3 minutes to achieve clear blue solutions, which were then diluted to 5.5 mg/mL in PBS (vehicle). These dosing solutions delivered 55 mg/mL when administered at 10 mL/kg (0.2 mL/20 g mouse), adjusted to the body weight of each animal. Unused stock and dosing solutions were stored protected from the light at −80° C. and returned to the client at the end of the study.

Treatment

On Day 1 of the study, mice bearing established subcutaneous MC38 xenografts were sorted into three treatment groups (n=10) and one group (n=1) that remained untreated. Dosing was initiated according to the treatment plan summarized in Table 1. Animals in Groups 1 and 2 were dosed once intravenously (i.v.) on Day 1 with a dosing volume of 10 mL/kg scaled to the body weights of each animal. Group 1 received PBS. Group 2 received 55 mg/kg D-Cy5. One animal (Group 3) remained untreated.

TABLE 1
Study design as of Day 1.

Treatment Regimen

| Group | n | Agent | mg/kg | Route | Schedule |
|---|---|---|---|---|---|
| 1 | 10 | PBS | — | iv | qd x 1 |
| 2 | 10 | D-Cy5 | 55 | iv | qd x 1 |
| 3 | 1 | No treatment | — | — | — |

Preparation of Tissues for Flow Cytometry

Mouse tumor samples were dissociated according to the manufacturer's instructions using the gentleMACS™ protocol “Tumor Dissociation Kit”. Briefly, tumors were excised and cut into small pieces (2-4 mm). Tumor samples were placed into an enzymatic buffer and processed on the gentleMACS™ Dissociator. Samples were incubated for 20 minutes at 37° C. with continuous rotation then filtered through a 70 micron cell strainer. Samples were washed twice in PBS containing 2.5% FBS to remove enzyme buffer, and the final single cell suspensions were prepared at 2×10⁷ cells/mL in PBS and kept on ice.

Flow Cytometry

100 μL of single cell suspensions were added into 96-well plates and washed once with PBS. Fc receptors were blocked using TruStain Fc (Biolegend) in 50 μL volume for five to ten minutes on ice prior to immunostaining. Next, 50 μL of Staining Buffer containing 2× concentration of antibodies (described in the protocol) was added to the sample for a total volume of 100 μL. The samples were gently pipetted up and down then stained for 30 minutes at 4° C. Cells were washed twice with 150 μL of Staining Buffer and resuspended in 100 μL of Staining Buffer. Countbright beads were prepared by briefly vortexing the beads and preparing a 1:3 dilution of the beads in Staining Buffer and resuspended in 100 μL of Staining. Isotype-control antibodies were used as negative staining controls when deemed necessary. For staining of internal markers, cells were permeabilized with 200 μL of Transcription Factor Fixation/Permeabilization buffer (eBioscience) for 30 minutes at 4° C. according to manufacturer's instructions. After two washes with 150 μL of Permeabilization Buffer (eBioscience), internal marker staining was carried out using antibodies diluted in 100 μL of Permeabilization Buffer for 30 minutes at 4° C. Cells were washed twice with 150 μL of Permeabilization Buffer and resuspended in 100 μL of Staining Buffer. All data were collected on a FortessaLSR (BD) and analyzed with FlowJo software (Tree Star, Inc., version 10.0.7r2). Cell populations were defined according to the protocol and the gating strategy was determined by initial gating on singlets (FSC-H vs. FSC-A), and then live cells based on Live/Dead Aqua viability staining. Antibodies used for staining target cell populations are summarized in Table 2.

TABLE 2
Antibody staining panel for CD4, CD8, T_{reg}, MDSC, and Macrophages.

| Cell Populations | Phenotypic Markers | Expression | Antibody Panel |
|---|---|---|---|
| CD4 | CD45⁺CD11b⁻CD3⁺CD4⁺CD8⁻ | Dendrimer- | CD45, CD3, CD4, |
| CD8 | CD45⁺CD11b⁻CD3⁺CD4⁻CD8⁺ | Cy5 | CD8, CD25, |
| T_{reg} | CD45⁺CD11b⁻ | | FoxP3*, CD11b, |
| | CD3⁺CD4⁺CD25⁺FoxP3⁺ | | F4/80, Ly6C, |
| mMDSC | CD45⁺ CD3⁻CD11b⁺F4/80⁻ | | Ly6G, CD206*, |
| | Ly6C^{hi}Ly6G⁻ | | Live/Dead |
| gMDSC | CD45⁺ CD3⁻CD11b⁺F4/80⁻ | | |
| | Ly6C^{lo}Ly6G⁺ | | |
| M1 Macrophage | CD45⁺F4/80⁺CD11b⁺CD206⁻ | | |
| M2 Macrophage | CD45⁺F4/80⁺CD11b⁺CD206⁺ | | |

*indicates internal marker

Ex Vivo Imaging

Excised tumors were imaged using the IVIS® SpectrumCT (Perkin Elmer, MA) equipped with a CCD camera (cooled at −90° C.), mounted on a light-tight specimen chamber with 640 nm excitation and 680 nm emission filters. Data were captured and quantitated in units of average radiant efficiency ([p/s/cm²]/[μW/cm²]), where p represents photons, s represents seconds and W represents watts. Data was analyzed using Living Image software 4.5.1. (Perkin Elmer, MA) and exported to Excel.

Toxicity

Animals were weighed on Days 1, 2 and 3 (the last day of the study). During this time the mice were observed for overt signs of any adverse, treatment-related (TR) side effects, and clinical signs were recorded when observed. Individual body weight (BW) was monitored, and any animal with weight loss exceeding 30% for one measurement or exceeding 25% for three consecutive measurements was euthanized as a TR death. Group mean body weight loss was also monitored according to CR Discovery Services protocol. Acceptable toxicity was defined as a group mean BW loss of less than 20% during the study and no more than 10% TR deaths. Any death was classified as TR if it was attributable to treatment side effects as evidenced by clinical signs and/or necropsy. A TR classification was also assigned to deaths by unknown causes within 14 days of the last dose. A death was classified as non-treatment-related (NTR) if there was no evidence that death was related to treatment side effects. NTR deaths were further categorized as follows: NTRa describes deaths due to accidents or human error; NTRm was assigned to deaths thought to result from tumor dissemination by invasion and/or metastasis based on necropsy results; NTRu describes deaths of unknown causes that lacked available evidence of death related to metastasis, tumor progression, accident or human error. It should be noted that treatment side effects cannot be excluded from deaths classified as NTRu.

Statistical and Graphical Analyses

Prism 8.0 (GraphPad) for Windows was used for graphical presentations and statistical analyses. Study groups experiencing toxicity beyond acceptable limits (>20% group mean body weight loss or greater than 10% treatment-related deaths) or having fewer than five evaluable observations, were not included in the statistical analysis. Note that tests of statistical significance do not provide an estimate of the magnitude of the difference between groups. Two-tailed statistical analyses were conducted at significance level P=0.05 and were not corrected for multiple comparisons.

### Results

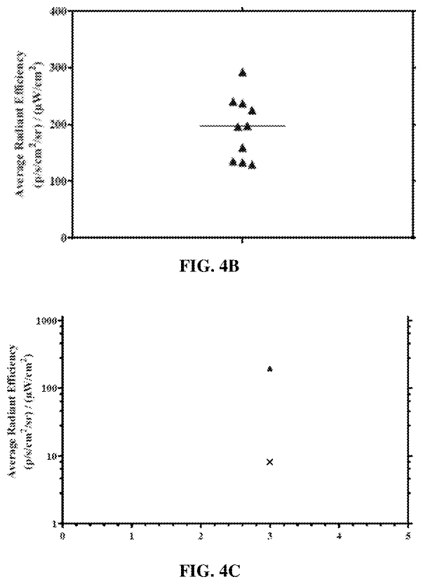

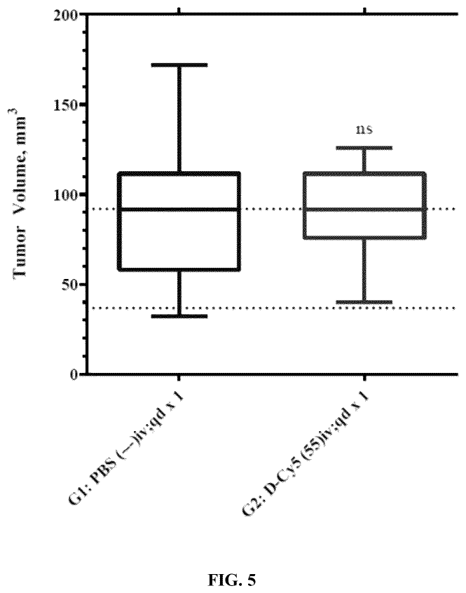

Day 3 individual average radiance efficiencies for Groups 1 and 2 were graphed in FIGS. 4A and 4B, with the mean of each group represented by a horizontal line. Group median average radiance efficiencies were plotted on log scales in FIG. 4C and evaluated statistically using the Kruskal-Wallis and Dunn's multiple comparisons tests. Box and whisker plots were constructed showing the Day 3 tumor volume data by group, with the “box” representing the 25th and 75th percentile of observations, the “line” representing the median of observations, and the “whiskers” representing the extreme observations (FIG. 5A). Median Tumor Volumes of three groups are summarized in Table 3. Statistical analyses of the differences between Day 3 median tumor volumes (MTVs) of control and treated groups were accomplished using the Mann-Whitney U test. Prism summarizes test results as not significant (ns) at P>0.05, significant (symbolized by “*”) at 0.01<P≤0.05, very significant (“**”) at 0.001<P≤0.01, and extremely significant (“***”) at P≤0.001. Tumor growth curves show group median tumor volumes as a function of time (FIG. 2B).

TABLE 3
Day 3 Median Tumor Volume (MTV).

| | MTV (n) | | Statistical |
|---|---|---|---|
| Group | Day 3 | % TGI | Significance |
| 1 | 92 (10) | — | |
| 2 | 92 (10) | 0 | ns |

Group mean body weight (BW) changes in the Female C57BL/6 mice during the three days of the study at Day 1, 2, and 3 post implantation of MC 38 cells were monitored as percent change±one standard error of the mean (SEM) from Day 1 No statistically significant group mean body weight losses were observed. No treatment related (TR) and non-treatment related (NTR) deaths were observed. No adverse events were observed during this three day study.

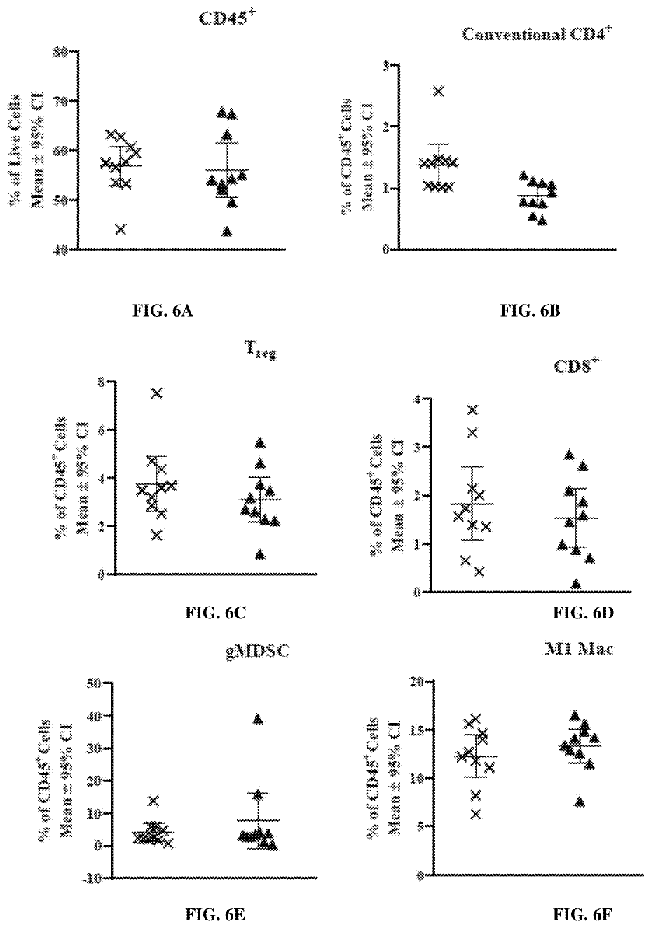

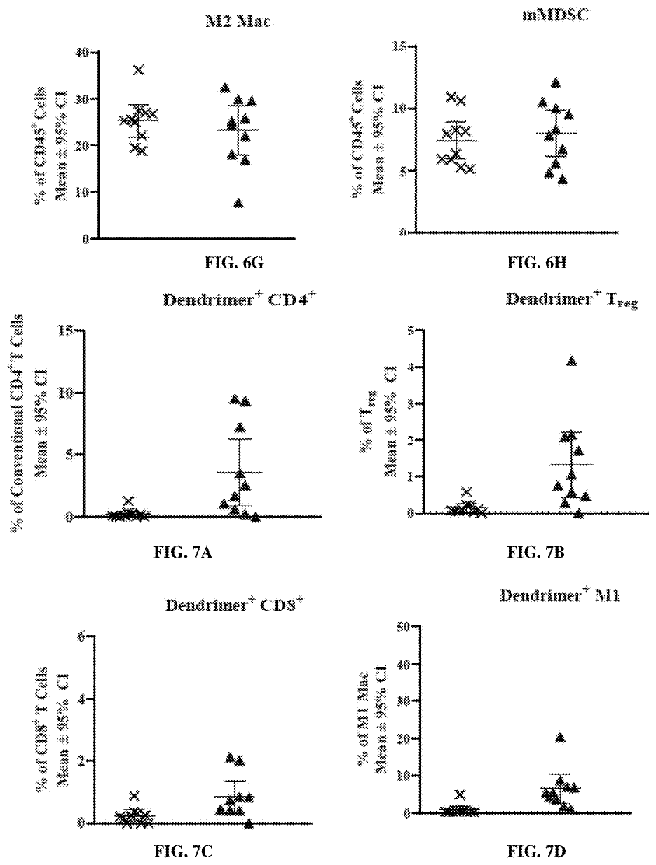

To characterize the immune profiles in all three groups, mouse tumor samples were analyzed using a panel of fluorescent-labeled antibodies as shown in Table 2. Cell types examined include CD4, Treg, CD8+, gMDSC, M1 macrophage, M2 macrophage and mMDSC population (FIGS. 6A-6H).

Table 4 summarizes CD45+ cell populations of total live cells in the processed tumor tissues at Day 3 in all three experimental groups. Tables 5 and 6 summarize different cell populations including conventional CD4, Treg, CD8+, gMDSC, M1 macrophage, M2 macrophage and mMDSC population percentages of CD45+ cells.

TABLE 4
CD45+ population percentages of total live cells in tumor tissues.

| | % of Parent Population | Statistical |
|---|---|---|
| Group | (% of Live Cells) | Significance (vs G1) |
| 1 | 56.89 ± 1.8 | — |
| 2 | 56.07 ± 2.5 | ns |

TABLE 5
Conventional CD4, Treg and CD8+ population
percentages of CD45+ cells.

| | Conventional | | |
|---|---|---|---|
| Group | CD4+ | Treg | CD8+ |
| 1 | 1.38 ± 0.1 | 3.76 ± 0.5 | 1.83 ± 0.3 |
| 2 | 0.87 ± 0.1 (*) | 3.12 ± 0.4 | 1.52 ± 0.3 |

Statistical Significance (vs G1), all non-significant except where indicated with
(*) P < 0.05.

TABLE 6
gMDSC, M1 macrophage, M2 macrophage and mMDSC
population percentages of CD45+ cells.

| | | M1 | M2 | |
|---|---|---|---|---|
| Group | gMDSC | macrophage | Macrophage | mMDSC |
| 1 | 4.14 ± 1.2 | 12.26 ± 1 | 25.35 ± 1.5 | 7.43 ± 0.7 |
| 2 | 7.67 ± 3.7 | 13.32 ± 0.8 | 23.22 ± 2.3 | 7.98 ± 0.8 |

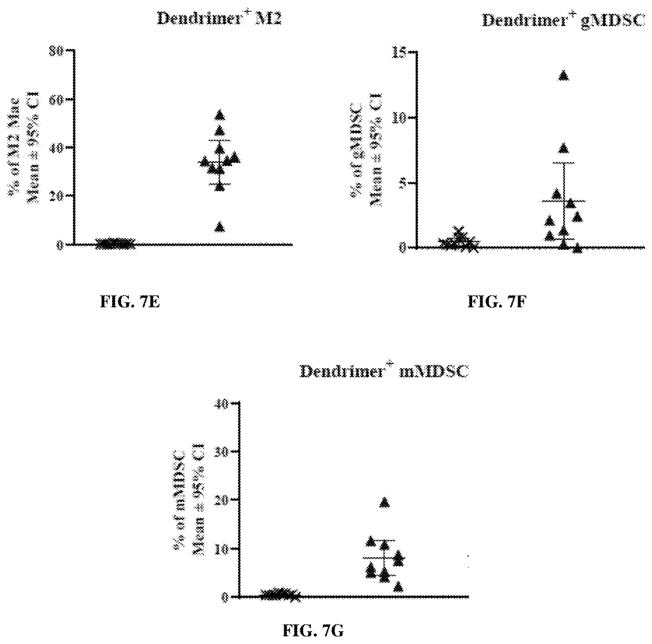

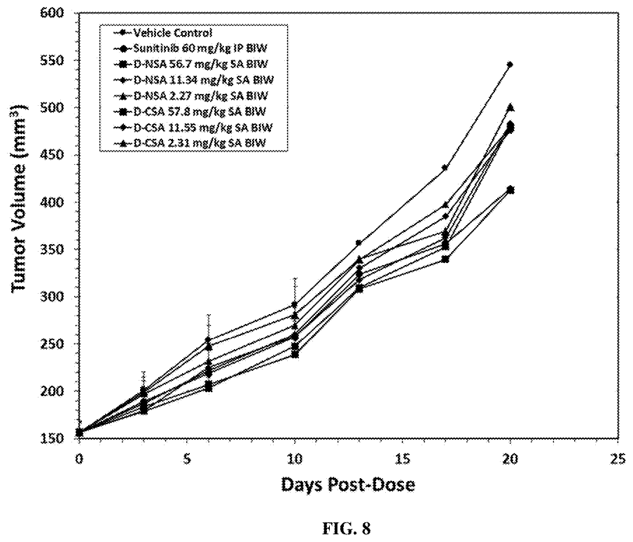

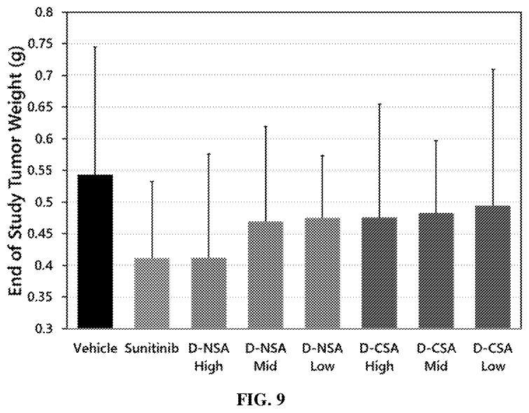

Dendrimer positive cells were also characterized in the processed tumor tissues at Day 3 in all three experimental groups (FIGS. 7A-7G). Tables 7 and 8 summarize different dendrimer-positive percentages of conventional CD4, Treg, CD8+, gMDSC, M1 macrophage, M2 macrophage and mMDSC cells.

TABLE 7
Dendrimer+ population percentages of conventional
CD4+, Treg and CD8+ cells.

| | Conventional | | |
|---|---|---|---|
| Group | CD4+ | Treg | CD8+ |
| 1 | 0.23 ± 0.1 | 0.14 ± 0.1 | 0.24 ± 0.1 |
| 2 | 3.56 ± 1.2 (*) | 1.33 ± 0.4 (**) | 0.83 ± 0.2 (ns) |

Statistical Significance:
(ns) = non-significant,
(*) = P < 0.05,
(**) = P ≤ 0.01,
*** = P ≤ 0.001, compared to group 1.

TABLE 8
Dendrimer+ population percentages of gMDSC,
M1 macrophage, M2 macrophage and mMDSC cells.

| | | M1 | M2 | |
|---|---|---|---|---|
| Group | gMDSC | macrophage | Macrophage | mMDSC |
| 1 | 0.45 ± 0.1 | 0.85 ± 0.5 | 0.32 ± 0.1 | 0.48 ± 0.1 |
| 2 | 3.58 ± 1.3 (*) | 6.47 ± 1.7 (*) | 34.02 ± 4 (***) | 8.1 ± 1.6 (***) |

Statistical Significance:
ns = non-significant,
(*) = P < 0.05,
(**) = P ≤ 0.01,
(***) = P ≤ 0.001, compared to group 1.

To evaluate the effect of hydroxyl dendrimer size and circulation time on targeting M2 tumor associated macrophages (TAMs), fluorescently tagged hydroxyl dendrimers of two types were generated, Generation 4 dendrimer (˜14,000 Da, 4 nm) conjugated with Cy5 (D4-Cy5) and Generation 6 dendrimer (˜58,000 Da, 7 nm) conjugated with VivoTag 680 (D6-V). A CSF1R tyrosine kinase inhibitor was also conjugated to the G6 hydroxyl dendrimer along with VivoTag 680 (C-D6-V). The syngeneic murine colon cancer line, MC38, was subcutaneously injected in C₅₇BL/6 mice (n=10/group) and tumors were allowed to grow to a minimum average size of 80-120 mm³. After tumor establishment, either D4-Cy5 or D6-V was injected IV (55 mg/kg, 10 mL/kg and mice were sacrificed 48 hrs post-dose (D4 and D6 are systemically cleared within 48 hr). Tumors were analyzed for total radiant fluorescence, FACS analysis for immune cell subpopulations, and immunohistochemistry. Analysis of total fluorescence indicated a greater tumor uptake of D6-V compared to D4-Cy5 consistent with previous studies. Selective uptake and retention was observed in M2 macrophage, M1 macrophage, and mMDSCs. Tumors included ˜56% CD45+ cells (Table 4), of which ˜20-25% were M2 macrophage, ˜13% were M1 macrophage, and ˜8% were mMDSCs (Table 6). 34±4% of all M2 macrophage, 6.5%±1.7% of all M1 macrophage, and 8.1±1.6% of all mMDSCs contained D4-Cy5 after the single IV dose (Table 8). The fraction of dendrimer in other immune cell populations including conventional CD4, Treg, CD8⁺ was less than 5% (Table 7). The C-D6-V had greater tumor uptake than D6-V suggesting that CSF1R binding further enhances tumor targeting as well as potentially impact M2 TAMs. These results indicate successful selective targeting of M2 TAMs and other tumor resident immune cells after systemic administration. Hydroxyl dendrimers provide a novel carrier for delivery of immune modulators to tumors while minimizing their systemic toxicity. Efficacy studies are ongoing to evaluate CSF1R inhibitors and other therapeutics conjugated to the hydroxyl dendrimers.

### Example 2: In Vivo Anti-Tumor Efficacy of Dendrimer-Bound Amide-Linked Sunitinib Analog (NSA) is Superior to the Ester-Linked Sunitinib Analog (CSA) in the Subcutaneous 786-O Human Renal Cancer Xenograft Model

The objective of this study was to evaluate in vivo anti-tumor efficacy of dendrimer-conjugated sunitinib analog in the treatment of the subcutaneous 786-O human renal cancer CDX model in female BALB/c nude mice.

### Methods

Cell Culture

The 786-O tumor cells (ATCC, cat #CRL-1932) were maintained in vitro as a monolayer culture in RPMI 1640 medium supplemented with 10% heat inactivated fetal bovine serum, 100 U/mL penicillin and 100 μg/mL streptomycin at 37° C. in an atmosphere of 5% CO2 in air. The tumor cells were routinely sub-cultured twice weekly by trypsin-EDTA treatment. The cells growing in an exponential growth phase were harvested and counted for tumor inoculation.

Animals

BALB/c nude, female, 6-8 weeks, weighing approximately 18-22 g. A total of 128 (64 plus 100%) used for the study, purchased from Shanghai SLAC Laboratory Animal Co., LTD. or other certified vendors.

Tumor Inoculation

Each mouse was inoculated subcutaneously 200 μl at the right flank with the 786-O cells (5×106) with 1:1 MATRIGEL® for tumor development. The animals were randomized and treatment started when the average tumor volume reached approximately 150-200 mm³ for the efficacy study. The test article administration and the animal numbers in each group are shown in Table 9:

TABLE 9
Experimental design

| | | | | Dosing | | |
|---|---|---|---|---|---|---|
| | | | Dose | Volume | Dosing | |
| Group | n | Treatment | (mg/kg) | (μL/g) | Route | Schedule |
| 1 | 8 | Vehicle | — | 10 | I.P | BIW × 3-4 W |
| | | control | | | | |
| | | (PBS) | | | | |
| 2 | 8 | Sunitinib | 60 | 10 | I.P | BIW × 3-4 W |
| | | maleate | | | | |
| 3 | 8 | D-NSA-high | 450 | 10 | I.P | BIW × 3-4 W |
| 4 | 8 | D-NSA-mid | 90 | 10 | I.P | BIW × 3-4 W |
| 5 | 8 | D-NSA-low | 18 | 10 | I.P | BIW × 3-4 W |
| 6 | 8 | D-CSA-high | 550 | 10 | I.P | BIW × 3-4 W |
| 7 | 8 | D-CSA-mid | 110 | 10 | I.P | BIW × 3-4 W |
| 8 | 8 | D-CSA-low | 22 | 10 | I.P | BIW × 3-4 W |

SA = Sunitinib analog
NSA = amide-linked sunitinib analog
CSA = ester-linked sunitinib analog
Low/mid/high = different amounts of active agent conjugated top dendrimers.

Before commencement of treatment, all animals were weighed and the tumor volumes measured. Since the tumor volume can affect the effectiveness of any given treatment, mice were assigned into groups using an Excel-based randomization software performing stratified randomization based upon their tumor volumes. This ensures that all the groups are comparable at the baseline.

The major endpoint is to see the tumor growth delayed or mice cured. Tumor sizes were measured twice weekly (or every other day) in two dimensions using a caliper, and the volume expressed in mm³ using the formula: V=0.5 a×b2 where a and b are the long and short diameters of the tumor, respectively. The tumor sizes are then used for the calculations of both T-C and T/C values. T-C is calculated with T as the median time (in days) required for the treatment group tumors to reach a predetermined size (e.g., 1,000 mm³), and C is the median time (in days) for the control group tumors to reach the same size. The T/C value (in percent) is an indication of antitumor effectiveness, T and C are the mean volume of the treated and control groups, respectively, on a given day.

### Results

The experiment assessed tumor growth in mice throughout the treatment period, to determine efficacy of the drug (sunitinib) delivered by the dendrimers. Tumor sizes (weight and volume) were measured. The results demonstrate that the sunitinib analog is effectively transferred to the site of RCC and reduces tumor volume (FIGS. 8 and 9). In addition, the data also demonstrate that the non-cleavable (amide) linked Sunitinib analog (NSA) is superior to the releasable (ester) linked (CSA) Sunitinib analog (FIGS. 8 and 9).

### Example 3: Systemic Administration of Hydroxyl Dendrimers to Target Inflammation in Arthritic Tissues

Chronic inflammation observed in arthritis and other autoimmune disorders is mediated primarily by pro-inflammatory reactive macrophages. Systemic administration of anti-inflammatory agents does not selectively target the affected tissue, or the reactive macrophages and often has significant side effects. Hydroxyl dendrimers have been observed to selectively target reactive macrophages and have been well tolerated in humans. Hydroxyl dendrimer-drug conjugates may provide a superior method for treating localized inflammation, from systemic administration.

### Methods

The binding affinity of the dendrimer-alendronate conjugate (D-ALN) (0.5 mg/ml in PBS) was evaluated against hydroxyapatite (HAP; 200 mg) at 37 degrees C., using UV/Vis spectrophotometry.

Lewis rats were immunized with an emulsion of type II bovine collagen in incomplete Freund's adjuvant intradermally on Day 1 and Day 7 to establish collagen-induced arthritis (CIA). Groups of CIA rats and naïve rats (N=5/group) were administered by IV (Single IV dose of 50 mg/kg HD-Cy5, ALN-HD-Cy5 or Vehicle on Day 19, with CIA induced on Day 1 & 7 with intradermal doses of type II bovine collagen in IFAon Day 19) either hydroxyl dendrimer labelled with Cy5 (D-Cy5), D-Cy5 conjugated with alendronate (ALN-D-Cy5), or vehicle control (see Table 10, below). On Day 21, animals were sacrificed for imaging of hind limbs, kidney and liver. Immunohistochemistry was also performed on hind limbs using CD68 (macrophages), CathK (osteoclasts) and DAPI.

TABLE 10
Experimental groups

| Groups | Set-up | Treatment |
|---|---|---|
| 1 | CIA | HD-Cy5 |
| 2 | CIA | ALN-HD-Cy5 |
| 3 | CIA | Vehicle |
| 4 | Naive | HD-Cy5 |
| 5 | Naive | ALN-HD-Cy5 |
| 6 | Naive | Vehicle |

Results

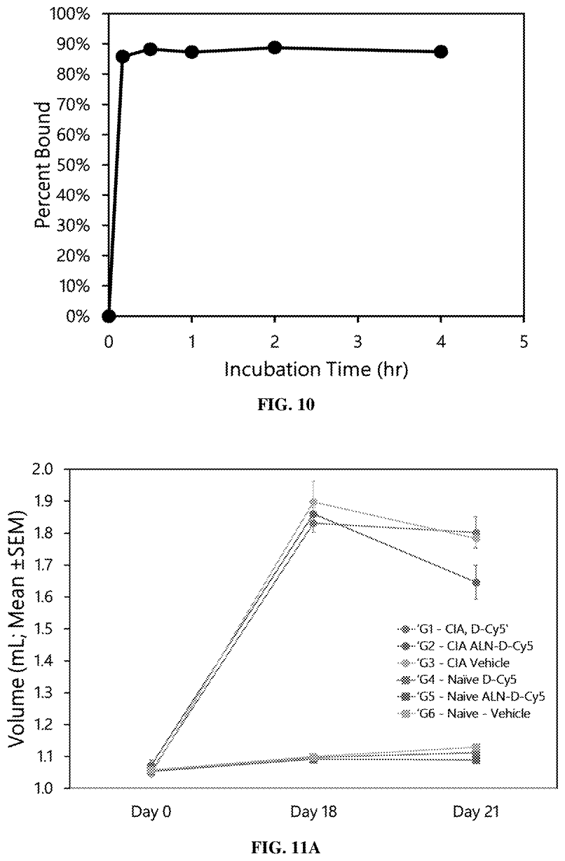

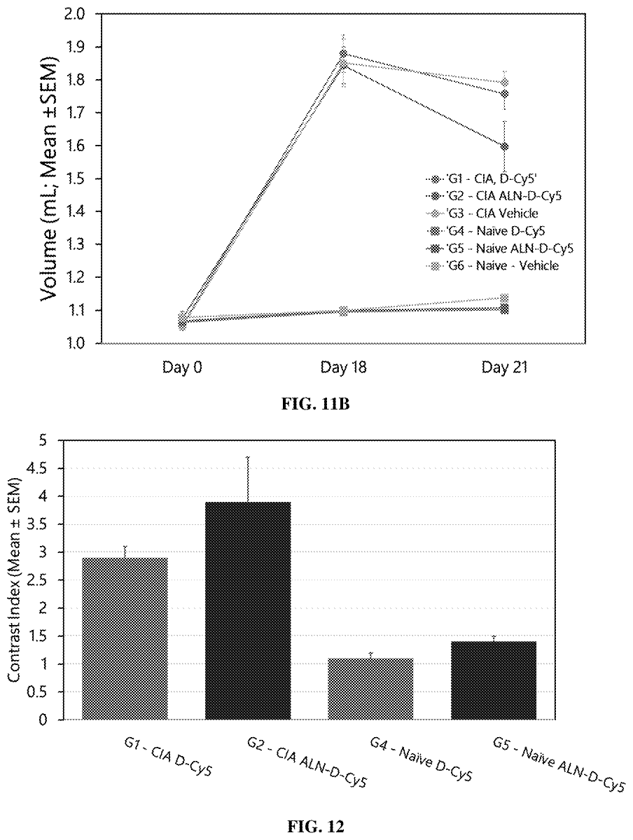

In vitro, D-ALN demonstrated strong binding affinity toward HAP with >85% of D-ALN bound to HAP in less than 10 minutes (FIG. 10). Upon intravenous administration, more than 100-fold greater radiant intensity from Cy5 was noted in the paw and knee joint of the CIA rats compared to the naïve rats, indicating significant selective uptake of the D-Cy5 into the regions of inflammation. While a comparable radiant intensity was noted in the joints of CIA rats treated with D-Cy5 or ALN-D-Cy5, a two-fold greater radiant intensity was noted in the paws for CIA rats treated with D-Cy5 (FIGS. 11A, 11B). A single dose of ALN-D-Cy5 reduced paw volumes by in CIA rats ˜10% after 2 days and clinical scores were comparable in all CIA groups (FIG. 12). Systemically administered HDs localize to arthritic tissues demonstrating selective targeting to reactive macrophage (HD-Cy5) and bone (ALN-HD-Cy5). Thus, HDs have been demonstrated to only be taken up by reactive inflammatory cells in animal models. Further, HDs are excreted intact in the urine in humans (Phase 1 study) and animals. HD therapeutics (HDTs) have thus been configured to deliver drugs specifically to arthritic tissues.

Together, the data demonstrate that systemically-administered hydroxyl dendrimer-drug conjugates localize to sites of inflammation in arthritic tissues. Alendronate, which binds bone, conjugated to the hydroxyl dendrimer appears to concentrate only in regions of the bone with potentially less uptake in reactive macrophages away from the bone.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A composition comprising a dendrimer complexed or conjugated with one or more immunomodulatory agents in an amount effective to suppress or inhibit one or more immunosuppressive cells associated with a tumor in a subject in need thereof.

2. The composition of claim 1, wherein the dendrimer is a hydroxyl-terminated dendrimer.

3. The composition of claim 1, wherein the dendrimer is a generation 4, generation 5, or generation 6 PAMAM dendrimer.

4. The composition of claim 1, wherein the immunomodulatory agent is glutaminase inhibitors.

5. The composition of claim 4, wherein the glutaminase inhibitor is selected from the group consisting of 6 diazo-5-oxo-L-norleucine (DON), azaserine, acivicin, and CB-839.

6. The composition of claim 1, wherein the immunomodulatory agent is covalently linked to the dendrimer, optionally via a linker or spacer moiety.

7. The composition of claim 6, wherein the immunomodulatory agent, or the linker or spacer moiety, or both the immunomodulatory agent and the linker or spacer moiety is bound to the dendrimer via a linkage selected from the group consisting of an ether, ester, and amide linkage, or combinations thereof.

8. The composition of claim 1, wherein the dendrimer is further complexed or conjugated with one or more chemotherapeutic agents.

9. The composition of claim 8, wherein the one or more chemotherapeutic agents are selected from the group consisting of amsacrine, bleomycin, busulfan, camptothecin, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epipodophyllotoxins, epirubicin, etoposide, etoposide phosphate, fludarabine, fluorouracil, gemcitabine, hydroxycarb amide, idarubicin, ifosfamide, innotecan, leucovorin, daunorubicin, lomustine, mechlorethamine, melphalan, mercaptopurine, mesna, methotrexate, mitomycin, mitoxantrone, oxaliplatin, paclitaxel, pemetrexed, pentostatin, procarbazine, raltitrexed, satraplatin, streptozocin, teniposide, tegafur-uracil, temozolomide, teniposide, thiotepa, tioguanine, topotecan, treosulfan, vinblastine, vincristine, vindesine, vinorelbine, vorinostat, taxol, trichostatin A and derivatives thereof, trastuzumab, cetuximab, rituximab, and bevacizumab.

10. The composition of claim 1, wherein the dendrimer is further complexed or conjugated with one or more diagnostic or labelling agents in an amount effective to diagnose or label the one or more immunosuppressive cells associated with a tumor in a subject in need thereof.

11. The composition of claim 9, wherein the immunosuppressive cells are myeloid-derived suppressor cells and/or tumor-associated macrophages (M2 macrophages).

12. A pharmaceutical composition comprising an effective amount of the composition of claim 1.

13. A method of treating a cancer comprising administering to a subject in need thereof an effective amount of the pharmaceutical composition of claim 12.

14. The method of claim 13, wherein the cancer is breast cancer, ovarian cancer, uterine cancer, prostate cancer, testicular germ cell tumor, brain cancer, gastric cancer, esophagus cancer, lung cancer, liver cancer, renal cell cancer and colon cancer.

15. The method of claim 13, wherein the effective amount is effective to reduce tumor size, and/or effective to enhance tumor-specific cytotoxic T cell responses in the subject.

16. The method of claim 13, further comprising administering to the subject one or more selected from the group consisting of an immune checkpoint modulator, a chemotherapeutic agent, an anti-infective agent, adoptive T cell therapy, a cancer vaccine, surgery, radiation therapy.

17. The method of claim 16, wherein the immune checkpoint modulator is selected from the group consisting of PD-1 antagonists, PD-1 ligand antagonists, and CTLA4 antagonists.

18. A composition comprising a dendrimer complexed or conjugated with one or more immunomodulatory agents in an amount effective to suppress or inhibit one or more pro-inflammatory cells associated with an inflammatory disease in a subject in need thereof.

19. (canceled)

20. (canceled)

21. The composition of claim 18, wherein the immunomodulatory agent is covalently linked to the dendrimer, optionally via a linker or spacer moiety.

22. The composition of claim 18, wherein the immunomodulatory agent, or the linker or spacer moiety, or both the immunomodulatory agent and the linker or spacer moiety is bound to the dendrimer via a linkage selected from the group consisting of an ether, ester, and amide linkage.

23. The composition of claim 18, wherein the dendrimer is further complexed or conjugated with one or more diagnostic or labelling agents in an amount effective to diagnose or label one or more pro-inflammatory cells associated with an autoimmune disease in a subject in need thereof.

24. The composition of claim 18, wherein the pro-inflammatory cells are pro-inflammatory macrophages (M1 macrophages).

25. A pharmaceutical composition comprising an effective amount of the composition of claim 18.

26. A method of treating an inflammatory disease comprising administering to a subject in need thereof an effective amount of the pharmaceutical composition of claim 18.

27. The method of claim 26, wherein the inflammatory disease is an autoimmune disease.

28. The method of claim 27, wherein the autoimmune diseases is selected from the group consisting of rheumatoid arthritis, psoriasis, psoriatic arthritis, systemic lupus erythematosus (SLE), type 1 diabetes, inflammatory bowel disease, and thyroid diseases.

29. The method of claim 26, wherein the inflammatory diseases is an inflammatory joint disease.

30. The method of claim 29, wherein the inflammatory joint disease is selected from the group consisting of osteoarthritis, rheumatoid arthritis, psoriatic arthritis, and juvenile arthritis.

31. A composition for treating a disease or disorder of the bone, comprising hydroxyl-terminated dendrimers complexed or conjugated with one or more therapeutic agents in an amount effective for treating one or more disorders of the bone, wherein the dendrimers are further covalently conjugated with alendronate.

32. The composition of claim 31, wherein the one or more therapeutic agents is covalently conjugated to the dendrimer, optionally via one or more linkers.

33. A method for treating a disease or disorder of the bone in a subject in need thereof, comprising administering to the subject in need thereof the composition of claim 31.
